(19) United States
(12) Patent Application Publication
(10) Pub. No.: US 2008/0176906 A1
(43) Pub. Date: Jul. 24, 2008

(54) Substituted Pyrrole Derivative

(75) Inventors: Nobuyuki Matsunaga, Osaka-shi (JP); Takenori Hitaka, Osaka-shi (JP)

Correspondence Address: EDWARDS ANGELL PALMER & DODGE LLP, P.O. BOX 55874, BOSTON, MA, 02205, US

(73) Assignee: Takeda Pharmaceutical Company Limited, Osaka (JP)

(21) Appl. No.: 11/793,113

(22) PCT Filed: Dec. 13, 2005
(86) PCT No.: PCT/JP2005/023215

§ 371 Date: Jun. 14, 2007

(30) Foreign Application Priority Data
Dec. 14, 2004 (JP) 2004-361641

(51) Int. Cl. A61K 31/4439; C07D 401/06; C07D 413/12; A61P 35/00; A61K 31/40; C07D 207/32

(52) U.S. Cl. 514/343; 546/276.4; 546/271.4; 548/566; 514/340; 514/427

(57) ABSTRACT

The present invention provides a novel pyrrole derivative having excellent androgen receptor antagonism, which is represented by the formula:

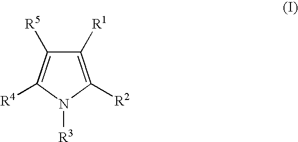

wherein R¹ represents a hydrogen atom, a cyano group or a group represented by the formula COOR^{A} (wherein R^{A} represents an optional substituted C₁₋₆ alkyl group), R² and R⁴ are the same or different, and each represents a hydrogen atom, a C₁₋₆ alkyl group, a C₃₋₆ cycloalkyl group, a trifluoromethyl group, an amino-C₁₋₆ alkyl group, a mono- or di-substituted amino-C₁₋₆ alkyl group, an optionally halogenated C₁₋₆ alkyl group substituted with an optionally substituted hydroxyl group, a C₂₋₆ alkenyl group substituted with an optionally substituted hydroxyl group, a C₁₋₆ alkyl group substituted with an optionally substituted and optionally oxidized thiol group, an optionally substituted with and optionally oxidized thiol group, a cyano group, an acyl group, an optionally substituted oxazolyl group or a 1,3-dioxolan-2-yl group, R³ represents a group represented by the formula:

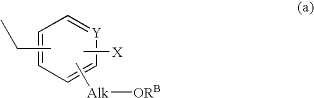

(wherein X represents a halogen atom, Y represents a carbon atom or a nitrogen atom, Alk represents an optionally substituted C₁₋₄ alkylene group, and R^{B} represents a hydrogen atom or an acyl group), and R⁵ represents a phenyl group which has a cyano group at a 4-position or a 3-position thereof, and may be further substituted, or a salt thereof. The present invention also provides an androgen receptor antagonist containing the pyrrole derivative, or a salt thereof.

### CROSS-REFERENCE TO RELATED APPLICATIONS

This application is the 35 U.S.C. §371 national stage of PCT application PCT/JP2005/023215, filed Dec. 13, 2005, which claims priority to Japanese patent application No. 2004-361641, filed Dec. 14, 2004. The contents of these applications are incorporated herein by reference in their entirety.

### TECHNICAL FIELD

The present invention relates to a novel pyrrole derivative and an androgen receptor antagonist comprising the same, more particularly, a novel pyrrole derivative having a preventing or treating effect of diseases depending on androgen, which is an androgenic hormone, by inhibiting an androgen receptor (AR), undergoing no influence by mutation, and exerting androgen receptor antagonism. The present invention also relates to an androgen receptor antagonist comprising the same.

### BACKGROUND ART

Journal of Medicinal Chemistry (1986), 29 (11), 2298-315 discloses a pyrrole derivative having an androgen receptor binding inhibitory activity. In addition, for example, WO 97/49709 and JP 11-255651 A disclose treatment of prostatic hypertrophy and the like by an androgen receptor binding inhibitor.

WO 03/57669 filed by the present applicant discloses an androgen receptor antagonist comprising a pyrrole derivative useful for preventing or treating prostate cancer which is one of most serious diseases among androgen-dependent diseases, and treating prostate cancer at a hormone independent term.

WO 02/02524 discloses a pyrrole derivative useful as an agent for treating AIDS.

### DISCLOSURE OF THE INVENTION

A main object of the present invention is to provide a pyrrole derivative which falls in a scope of a pyrrole derivative disclosed in the above WO 03/57669, but is novel and exerts excellent androgen receptor antagonism.

The present inventors intensively studied a pyrrole derivative having androgen receptor antagonism, and found out that a novel pyrrole derivative having a structure in which a nitrogen atom of the pyrrole ring is substituted with a specific substituted benzyl group or substituted pyridylmethyl group, specifically, with a benzyl group or a pyridylmethyl group having a substituent represented by -Alk-OR^{B} (each symbol is as defined hereinafter) has unexpectedly excellent androgen receptor antagonism, excellent pharmacokinetics and the like, and further, has low toxicity, which resulted in completion of the present invention.

That is, the present invention provides:

(1) A compound represented by the formula:

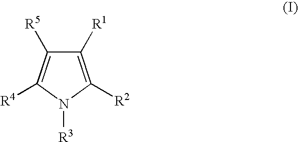

wherein R¹ represents a hydrogen atom, a cyano group or a group represented by the formula COOR^{A} (wherein R^{A} represents an optionally substituted C₁₋₆ alkyl group), R² and R⁴ are the same or different, and each represents a hydrogen atom, a C₁₋₆ alkyl group, a C₃₋₆ cycloalkyl group, a trifluoromethyl group, an amino-C₁₋₆ alkyl group, a mono- or di-substituted amino-C₁₋₆ alkyl group, an optionally halogenated C₁₋₆ alkyl group substituted with an optionally substituted hydroxyl group, a C₂₋₆ alkenyl group substituted with an optionally substituted hydroxyl group, a C₁₋₆ alkyl group substituted with an optionally substituted and optionally oxidized thiol group, an optionally substituted and optionally oxidized thiol group, a cyano group, an acyl group, an optionally substituted oxazolyl group or a 1,3-dioxolan-2-yl group, R³ represents a group represented by the formula:

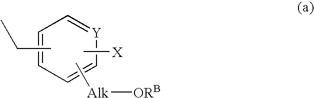

(wherein X represents a halogen atom, Y represents a carbon atom or a nitrogen atom, Alk represents an optionally substituted C₁₋₄ alkylene group, and R^{B} represents a hydrogen atom or an acyl group), and R⁵ represents a phenyl group which has a cyano group at a 4-position or a 3-position thereof and may be further substituted (hereinafter referred to as the compound (I)), or a salt thereof;

(2) The compound according to the above (1), wherein R¹ is a cyano group;

(3) The compound according to the above (1), wherein R² is a C₁₋₆ alkyl group, a C₃₋₆ cycloalkyl group, a trifluoromethyl group, an amino-C₁₋₆ alkyl group, a mono- or di-substituted amino-C₁₋₆ alkyl group, a C₁₋₆ alkyl group substituted with an optionally substituted hydroxyl group, a C₁₋₆ alkyl group substituted with an optionally substituted and optionally oxidized thiol group, an optionally substituted and optionally oxidized thiol group, a cyano group or an acyl group;

(4) The compound according to the above (1), wherein R² is a C₁₋₆ alkyl group, a C₃₋₆ cycloalkyl group, a trifluoromethyl group, an amino-C₁₋₆ alkyl group, a mono- or di-substituted amino-C₁₋₆ alkyl group, a C₁₋₆ alkyl group substituted with an optionally substituted hydroxyl group, a C₁₋₆ alkyl group substituted with an optionally substituted and optionally oxidized thiol group, a cyano group or an acyl group;

(5) The compound according to the above (3), wherein R² is a methyl group, an ethyl group, a propyl group, an isopropyl group, a cyclopropyl group, a trifluoromethyl group, a mono- or di-substituted amino-methyl group, a hydroxymethyl group, a hydroxyethyl group, a hydroxyisopropyl group, a C₁₋₆ alkyloxymethyl group, a C₁₋₆ alkylthio group, a C₁₋₆ alkylsulfonyl group, a C₁₋₆ alkylthiomethyl group, a C₁₋₆ alkylsulfonylmethyl group, an acetyl group, a carbamoyl group or a mono- or di-substituted carbamoyl group;

(6) The compound according to the above (3), wherein R² is a methyl group, an ethyl group, a propyl group, an isopropyl group, a cyclopropyl group, a trifluoromethyl group, a mono- or di-substituted amino-methyl group, a hydroxymethyl group, a hydroxyethyl group, a hydroxyisopropyl group, a C₁₋₆ alkyloxymethyl group, a C₁₆ alkylthiomethyl group, a C₁₋₆ alkylsulfonylmethyl group, an acetyl group, a carbamoyl group or a mono- or di-substituted carbamoyl group;

(7) The compound according to the above (1), wherein R³ is a group represented by the formula:

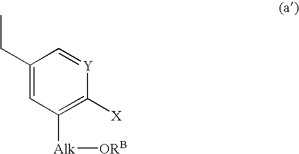

wherein each symbol is as defined in the above (1);

(8) The compound according to the above (7), wherein X is a chlorine atom or a fluorine atom;

(9) The compound according to the above (7), wherein R^{B} is a hydrogen atom, a succinoyl group or a dimethylaminomethylcarbonyl group;

(10) The compound according to the above (7), wherein Alk is a methylene group;

(11) The compound according to the above (1), wherein R⁴ is a C₁₋₆ alkyl group, a C₃₋₆ cycloalkyl group, a trifluoromethyl group, an amino-C₁₋₆ alkyl group, a mono- or di-substituted amino-C₁₋₆ alkyl group, a C₁₋₆ alkyl group substituted with an optionally substituted hydroxyl group, a C₁₋₆ alkyl group substituted with an optionally substituted and optionally oxidized thiol group, an optionally substituted and optionally oxidized thiol group, a cyano group or an acyl group;

(12) The compound according to the above (1), wherein R⁴ is a methyl group, an ethyl group, a propyl group, an isopropyl group, a cyclopropyl group, a trifluoromethyl group, a mono- or di-substituted amino-methyl group, a hydroxymethyl group, a hydroxyethyl group, a hydroxyisopropyl group, a C₁₋₆ alkyloxymethyl group, a C₁₋₆ alkylthio group, a C₁₋₆ alkylsulfonyl group, a C₁₋₆ alkylthiomethyl group, a C₁₋₆ alkylsulfonylmethyl group, an acetyl group, a carbamoyl group or a mono- or di-substituted carbamoyl group;

(13) The compound according to the above (1), wherein R⁵ is a 4-cyanophenyl group, a 3-cyanophenyl group, or a 4-cyano-3-(trifluoromethyl)phenyl group;

(14) The compound according to the above (1), wherein R¹ is a hydrogen atom, a cyano group or a C₁₋₆ alkoxycarbonyl group,

R² is (i) a hydrogen atom,

(ii) an optionally halogenated C₁₋₆ alkyl group,

(iii) a C₃₋₆ cycloalkyl group,

(iv) a trifluoromethyl group,

(v) a C₁₋₆ alkyl group substituted with an amino mono- or di-substituted with C₁₋₆ alkyl,

(vi) an optionally halogenated C₁₋₆ alkyl group substituted with a hydroxyl group optionally substituted with C₁₋₆ alkyl,

(vii) a C₂₋₆ alkenyl group substituted with a hydroxyl group,

(viii) a C₁₋₆ alkyl group substituted with an optionally oxidized thiol group optionally substituted with C₁₋₆ alkyl,

(ix) an optionally oxidized thiol group optionally substituted with C₁₋₆ alkyl,

(x) a cyano group,

(xi) a C₁₋₆ alkanoyl group,

(xii) an oxazolyl group, or

(xiii) a 1,3-dioxolan-2-yl group,

Alk is a C₁₋₄ alkylene group optionally substituted with an optionally halogenated C₁₋₆ alkyl,

R^{B} is (i) a hydrogen atom, (ii) a carbamoyl group, (iii) a carboxy-C₁₋₆ alkyl-carbonyl group or (iv) a mono- or di-C₁₋₆ alkylamino-C₁₋₆ alkyl-carbonyl group,

X is a halogen atom,

Y is a carbon atom or a nitrogen atom,

R⁴ is a C₁₋₆ alkyl group optionally substituted with a hydroxyl group, a trifluoromethyl group, or a cyano group,

R⁵ is a phenyl group which has a cyano group at a 4-position or a 3-position thereof, and may be substituted with an optionally halogenated C₁₋₆ alkyl;

(15) The compound according to the above (1), wherein R¹ is a hydrogen atom, a cyano group or a methoxycarbonyl group, R² is a methyl group, an ethyl group, a propyl group, an isopropyl group, a cyclopropyl group, a trifluoromethyl group, a 1-hydroxyethyl group or an acetyl group, R³ is a 6-chloro-5-(hydroxymethyl)pyridin-3-ylmethyl group, a 6-chloro-5-(1-hydroxy-2,2,2-trifluoroethyl)pyridin-3-ylmethyl group, a 4-chloro-3-(1-hydroxy-2,2,2-trifluoroethyl)benzyl group, a 4-chloro-3-(acetoxymethyl)benzyl group or a 4-chloro-3-(hydroxymethyl)benzyl group, R⁴ is a cyano group, a methyl group or an ethyl group, and R⁵ is a 4-cyano-2-fluorophenyl group or a 4-cyanophenyl group;

(16) The compound according to the above (1), wherein R¹ is a hydrogen atom, a cyano group or a methoxycarbonyl group, R² is a methyl group, an ethyl group, a propyl group, a cyclopropyl group or a trifluoromethyl group, R³ is a 6-chloro-5-(hydroxymethyl)pyridin-3-ylmethyl group, a 6-chloro-5-(1-hydroxy-2,2,2-trifluoroethyl)pyridin-3-ylmethyl group, a 4-chloro-3-(1-hydroxy-2,2,2-trifluoroethyl)benzyl group, a 4-chloro-3-(acetoxymethyl)benzyl group or a 4-chloro-3-(hydroxymethyl)benzyl group, R⁴ is a cyano group, a methyl group or an ethyl group, and R⁵ is a 4-cyano-2-fluorophenyl group or a 4-cyanophenyl group;

(17) A prodrug of the compound according to the above (1);

(18) (i) 1-{[6-Chloro-5-(hydroxymethyl)pyridin-3-yl]methyl}-4-(4-cyanophenyl)-2,5-dimethyl-1H-pyrrole-3-carbonitrile,

(ii) 1-{[6-chloro-5-(hydroxymethyl)pyridin-3-yl]methyl}-4-(4-cyanophenyl)-2-ethyl-5-methyl-1H-pyrrole-3-carbonitrile,

(iii) 1-{[6-chloro-5-(hydroxymethyl)pyridin-3-yl]methyl}-4-(4-cyanophenyl)-5-methyl-2-propyl-1H-pyrrole-3-carbonitrile,

(iv) 1-{[6-chloro-5-(hydroxymethyl)pyridin-3-yl]methyl}-4-(4-cyanophenyl)-2-cyclopropyl-5-methyl-1H-pyrrole-3-carbonitrile,

(v) 1-{[6-chloro-5-(hydroxymethyl)pyridin-3-yl]methyl}-4-(3-cyanophenyl)-2,5-dimethyl-1H-pyrrole-3-carbonitrile,

(vi) 1-[4-chloro-3-(hydroxymethyl)benzyl]-4-(4-cyanophenyl)-2,5-dimethyl-1H-pyrrole-3-carbonitrile,

(vii) 2-chloro-5-{[3-cyano-4-(4-cyanophenyl)-2,5-dimethyl-1H-pyrrol-1-yl]methyl}benzyl N,N-dimethylglycinate,

(viii) 4-[(2-chloro-5-{[3-cyano-4-(4-cyanophenyl)-2,5-dimethyl-1H-pyrrol-1-yl]methyl}benzyl)oxy]-4-oxobutanoic acid,

(ix) 4-[(2-chloro-5-{[3-cyano-4-(4-cyanophenyl)-5-methyl-2-propyl-1H-pyrrol-1-yl]methyl}pyridin-3-yl)methoxy]-4-oxobutanoic acid,

(x) 4-[(2-chloro-5-{[3-cyano-4-(4-cyanophenyl)-2-ethyl-5-methyl-1H-pyrrol-1-yl]methyl}pyridin-3-yl)methoxy]-4-oxobutanoic acid,

(xi) 4-[(2-chloro-5-{[3-cyano-4-(4-cyanophenyl)-2-cyclopropyl-5-methyl-1H-pyrrol-1-yl]methyl}pyridin-3-yl)methoxy]-4-oxobutanoic acid,

(xii) 1-{[6-chloro-5-(2,2,2-trifluoro-1-hydroxyethyl)pyridin-3-yl]methyl}-4-(4-cyanophenyl)-2,5-dimethyl-1H-pyrrole-3-carbonitrile,

(xiii) 4-(1-{[6-chloro-5-(hydroxymethyl)pyridin-3-yl]methyl}-2,5-dimethyl-1H-pyrrol-3-yl)benzonitrile,

(xiv) 4-(1-{[6-chloro-5-(hydroxymethyl)pyridin-3-yl]methyl}-5-cyclopropyl-2-methyl-1H-pyrrol-3-yl)benzonitrile, or

(xv) 1-{[6-chloro-5-(hydroxymethyl)pyridin-3-yl]methyl}-3-(4-cyanophenyl)-5-methyl-1H-pyrrole-2-carbonitrile, or a salt thereof;

(19) A process for preparing the compound according to the above (1) or a salt thereof, which comprises the steps of:

subjecting a compound represented by the formula:

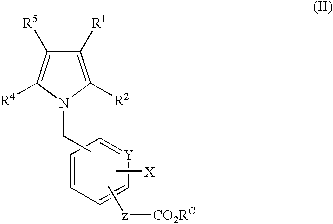

wherein Z represents a bond or an optionally substituted C₁₋₃ alkylene group, R^{C} represents an optionally substituted C₁₋₆ alkyl group, and the other symbols are as defined in the above (1), or a salt thereof to a reducing reaction to obtain a compound represented by the formula:

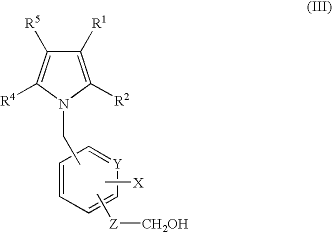

wherein the symbols are as defined above, or a salt thereof and, if necessary, subjecting it to a functional group-converting reaction;

(20) A medicament comprising the compound (I), or a salt or a prodrug thereof;

(21) The medicament according to the above (20), which is an androgen receptor antagonist;

(22) The medicament according to the above (20), wherein the androgen receptor is a normal androgen receptor and/or a mutated androgen receptor;

(23) The medicament according to the above (20), which is an agent for preventing or treating hormone-sensitive cancer at an androgen dependent term and/or at an androgen independent term;

(24) The medicament according to the above (20), which is an agent for preventing or treating prostate cancer;

(25) A medicament which comprises a combination of the compound (I), or a salt or a prodrug thereof, and an anti-cancer drug;

(25 a) A medicament which comprises the compound (I), or a salt or a prodrug thereof to be used in combination with an anti-cancer drug;

(26) The medicament according to the above (25) or (25 a), wherein the anti-cancer drug is an LH-RH derivative;

(27) A method for antagonizing an androgen receptor of a mammal which comprises administering an effective amount of the compound (I), or a salt or prodrug thereof to the mammal;

(28) A method for preventing or treating hormone-sensitive cancer at an androgen dependent term and/or at an androgen independent term, which comprises administering an effective amount of the compound (I), or a salt or prodrug thereof to a mammal;

(29) A method of preventing or treating prostate cancer, which comprises administering an effective amount of the compound (I), or a salt or prodrug thereof to a mammal;

(30) The method according to the above (28) or (29), wherein an effective amount of an anti-cancer drug is further administered;

(31) The method according to the above (30), wherein the anti-cancer drug is an LH-RH derivative;

(32) Use of the compound (I), or a salt or prodrug thereof for manufacturing an androgen receptor antagonist;

(33) Use of the compound (I), or a salt or prodrug thereof for manufacturing an agent for preventing or treating hormone-sensitive cancer at an androgen dependent term and/or at an androgen independent term;

(34) Use of the compound (I), or a salt or prodrug thereof for manufacturing an agent for preventing or treating prostate cancer;

(35) The use according to the above (33) or (34), wherein an anti-cancer drug is further used;

(36) The use of the above (35), wherein the anti-cancer drug is an LH-RH derivative; and the like.

The present invention further provides:

(37) the compound according to the above (1), wherein R¹ is

(i) a hydrogen atom,

(ii) a cyano group, or

(iii) a group represented by the formula COOR² wherein R^{A}. represents a C₁₋₆ alkyl group optionally substituted with a substituent selected from a substituent A group [a group consisting of oxo, halogen atom, C₁₋₃ alkylenedioxy, nitro, cyano, optionally halogenated C₁₋₆ alkyl, optionally halogenated C₂₋₆ alkenyl, carboxy C₂₋₆ alkenyl, optionally halogenated C₂₋₆ alkynyl, optionally halogenated C₃₋₆ cycloalkyl, C₆₋₁₄ aryl, optionally halogenated C₁₋₈ alkoxy, C₁₋₆ alkoxy-carbonyl-C₁₋₆ alkoxy, hydroxy, C₆₋₁₄ aryloxy, C₇₋₁₆ aralkyloxy, mercapto, optionally halogenated C₁₋₆ alkylthio, C₆₋₁₄ arylthio, C₇₋₁₆ aralkylthio, amino, mono-C₁₋₆ alkylamino, mono-C₆₋₁₄ arylamino, di-C₁₋₆ alkylamino, di-C₆₋₁₄ arylamino, formyl, carboxy, carboxy-C₂₋₆ alkenyl, carboxy-C₁₋₆ alkyl, C₁₋₆ alkyl-carbonyl, C₃₋₆ cycloalkyl-carbonyl, C₁₋₆ alkoxy-carbonyl, C₆₋₁₄ aryl-carbonyl, C₇₋₁₆ aralkyl-carbonyl, C₆₋₁₄ aryloxy-carbonyl, C₇₋₁₆ aralkyloxy-carbonyl, 5- or 6-membered heterocyclic-carbonyl containing 1 to 3 heteroatoms selected from a nitrogen atom, an oxygen atom, an oxygen atom and the like in addition to carbon atoms, carbamoyl, thiocarbamoyl, mono-C₁₋₆ alkyl-carbamoyl, di-C₁₋₆ alkyl-carbamoyl, mono- or di-C₆₋₁₄ aryl-carbamoyl, 5- or 6-membered heterocyclic carbamoyl containing 1 to 3 heteroatoms selected from a nitrogen atom, an oxygen atom, an oxygen atom and the like in addition to carbon atoms, C₁₋₆ alkylsulfonyl, C₁₋₆ alkylsulfinyl, C₆₋₁₄ arylsulfonyl, C₆₋₁₄ arylsulfinyl, formylamino, C₁₋₆ alkyl-carbonylamino, C₆₋₁₄ aryl-carbonylamino, C₁₋₆ alkoxy-carbonylamino, C₁₋₆ alkylsulfonylamino, C₆₋₁₄ arylsulfonylamino, C₁₋₆ alkyl-carbonyloxy, C₆₋₁₄ aryl-carbonyloxy, C₁₋₆ alkoxy-carbonyloxy, mono-C₁₋₆ alkyl-carbamoyloxy, di-C₁₋₆ alkyl-carbamoyloxy, C₆₋₁₄ aryl-carbamoyloxy, nicotinoyloxy, optionally substituted 5- to 7-membered saturated cyclic amino (the substituent is selected from halogen atom, optionally halogenated C₁₋₆ alkyl, optionally halogenated C₃₋₆ cycloalkyl, C₆₋₁₄ aryl and optionally halogenated C₁₋₈ alkoxy), a 5- to 10-membered aromatic heterocyclic group containing 1 to 3 heteroatoms selected from a nitrogen atom, an oxygen atom, an oxygen atom and the like in addition to carbon atoms, and sulfo],

R² and R⁴ are the same or different, and each represents

(i) a hydrogen atom,

(ii) a C₁₋₆ alkyl group,

(iii) a C₃₋₆ cycloalkyl group,

(iv) a trifluoromethyl group,

(v) an amino-C₁₋₆ alkyl group,

(vi) a mono- or di-substituted amino-C₁₋₆ alkyl group,

(vii) an optionally halogenated C₁₋₆ alkyl group substituted with a hydroxyl group optionally substituted with a substituent selected from the group consisting of a C₁₋₆ alkyl group optionally substituted with a substituent selected from the substituent A group, a C₂₋₆ alkenyl group optionally substituted with a substituent selected from the substituent A group, a C₂₋₆ alkynyl group optionally substituted with a substituent selected from the substituent A group, a C₃₋₆ cycloalkyl group optionally substituted with a substituent selected from the substituent A group, a C₆₋₁₄ aryl group optionally substituted with a substituent selected from the substituent A group, and a C₇₋₁₆ aralkyl group optionally substituted with a substituent selected from the substituent A group (hereinafter referred to as the substituent B group),

(viii) a C₂₋₆ alkenyl group substituted with a hydroxyl group optionally substituted with a substituent selected from the substituent B group,

(ix) a C₁₋₆ alkyl group substituted with a group represented by the formula-S(O)ₙR⁶ (wherein R⁶ represents a hydrogen atom, or a group selected from the substituent B group, and n represents 0, 1 or 2),

(x) a group represented by the formula —S(O)ₙR⁶ (wherein R⁶ represents a hydrogen atom, a group selected from the substituent B group, and n represents 0, 1 or 2),

(xi) a cyano group,

(xii) an acyl group represented by the formula: —COOR⁷, —CONR⁸R⁹, —COR¹⁰ or —(C═S)—NR¹¹R¹² (wherein R⁷, R⁸, R⁹, R¹⁰, R¹¹ and R¹² represent a hydrogen atom or a group selected from the substituent B group, respectively, and R⁸ and R⁹, R¹¹ and R¹² may be taken together with the adjacent nitrogen atom to form a C₃₋₆ cycloalkyl group optionally having a substituent selected from the substituent B group, or a 5- or 6-membered heterocyclic group containing 1 to 3 heteroatoms selected from a nitrogen atom, a sulfur atom, and an oxygen atom in addition to carbon atoms optionally having a substituent selected from the substituent B group, respectively),

(xiii) an oxazolyl group optionally substituted with a substituent selected from the substituent A group, or

(xiv) a 1,3-dioxolan-2-yl group,

R³ represents a group represented by the formula:

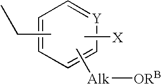

(wherein X represents a halogen atom,

Y represents a carbon atom or a nitrogen atom,

Alk represents a C₁₋₄ alkylene group optionally substituted with a substituent selected from the substituent A group,

R^{B} represents a hydrogen atom, or an acyl group represented by the formula: —COOR⁷, —CONR⁸R⁹, —COR¹⁰ or —(C═S)—NR¹¹R¹²

(wherein R⁷, R⁸, R⁹, R¹⁰, R¹ and R¹² represent a hydrogen atom or a group selected from the substituent B group, respectively, and R⁸ and R⁹, and R¹¹ and R¹² may be taken together with the adjacent nitrogen atom to form a C₃₋₆ cycloalkyl group optionally having a substituent selected from the substituent B group, or a 5- or 6-membered heterocyclic group containing 1 to 3 heteroatoms selected from a nitrogen atom, a sulfur atom and an oxygen atom in addition to carbon atoms optionally having a substituent selected from the substituent B group, respectively)), and

R⁵ represents a phenyl group which has a cyano group at a 4-position or 3-position thereof, and may be substituted with a substituent selected from the substituent A group.

When the compound (I) or a salt thereof contains an asymmetric carbon in its structure, all of the optically active isomers and racemates are included in the scope of the present invention, and these compounds or a salt thereof may be any of a hydrate and an anhydride.

The compound (I) of the present invention or a salt thereof not only exhibits a strong antagonist activity to a natural type, androgen receptor, but also exhibits high antagonism to a mutated androgen receptor, and these compounds can be orally administered, and are useful as a medicament which has extremely low toxicity, has androgen receptor antagonism, and has efficacy, for example, even for prostate cancer at a hormone independent term.

### BEST MODE FOR CARRYING OUT THE INVENTION

In the compound (I), R¹ represents a hydrogen atom, a cyano group or a group represented by the formula COOR^{A}, and R^{A} represents an optionally substituted C₁₋₆ alkyl group.

Examples of a C₁₋₆ alkyl group include methyl, ethyl, propyl, isopropyl, butyl, isobutyl, sec-butyl, tert-butyl, pentyl, hexyl, etc.

Examples of the substituent of the C₁₋₆ alkyl group include oxo, a halogen atom (e.g. fluorine, chlorine, bromine, iodine, etc.), C₁₋₃ alkylenedioxy (e.g. methylenedioxy, ethylenedioxy, etc.), nitro, cyano, optionally halogenated C₁₋₆ alkyl, optionally halogenated C₂₋₆ alkenyl, carboxy C₂₋₆ alkenyl (e.g. 2-carboxyethenyl, 2-carboxy-2-methylethenyl, etc.), optionally halogenated C₂₋₆ alkynyl, optionally halogenated C₃₋₆ cycloalkyl, C₆₋₁₄ aryl (e.g. phenyl, 1-naphthyl, 2-naphthyl, 2-biphenylyl, 3-biphenylyl, 4-biphenylyl, 2-anthryl, etc.), optionally halogenated C₁₋₈ alkoxy, C₁₋₆ alkoxy-carbonyl-C₁₋₆ alkoxy (e.g. ethoxycarbonylethyloxy, etc.), hydroxy, C₆₋₁₄ aryloxy (e.g. phenyloxy, 1-naphthyloxy, 2-naphthyloxy, etc.), C₇₋₁₆ aralkyloxy (e.g. benzyloxy, phenethyloxy, etc.), mercapto, optionally halogenated C₁₋₆ alkylthio, C₆₋₁₄ arylthio (e.g. phenylthio, 1-naphthylthio, 2-naphthylthio, etc.), C₇₋₁₆ aralkylthio (e.g. benzylthio, phenethylthio, etc.), amino, mono-C₁₋₆ alkylamino (e.g. methylamino, ethylamino, etc.), mono-C₆₋₁₄ arylamino (e.g. phenylamino, 1-naphthylamino, 2-naphthylamino, etc.), di-C₁₋₆ alkylamino (e.g. dimethylamino, diethylamino, ethylmethylamino, etc.), di-C₆₋₁₄ arylamino (e.g. diphenylamino, etc.), formyl, carboxy, carboxy-C₂₋₆ alkenyl, carboxy-C₁₋₆ alkyl, C₁₋₆ alkyl-carbonyl (e.g. acetyl, propionyl, etc.), C₃₋₆ cycloalkyl-carbonyl (e.g. cyclopropylcarbonyl, cyclopentylcarbonyl, cyclohexylcarbonyl, etc.), C₁₋₆ alkoxy-carbonyl (e.g. methoxycarbonyl, ethoxycarbonyl, propoxycarbonyl, tert-butoxycarbonyl, etc.), C₆₋₁₄ aryl-carbonyl (e.g. benzoyl, 1-naphthoyl, 2-naphthoyl, etc.), C₇₋₁₆ aralkyl-carbonyl (e.g. phenylacetyl, 3-phenylpropionyl, etc.), C₆₋₁₄ aryloxy-carbonyl (e.g. phenoxycarbonyl, etc.), C₇₋₁₆ aralkyloxy-carbonyl (e.g. benzyloxycarbonyl, phenethyloxycarbonyl, etc.), 5- or 6-membered heterocyclic-carbonyl (e.g. nicotinoyl, isonicotinoyl, thenoyl, furoyl, morpholinocarbonyl, thiomorpholinocarbonyl, piperazine-1-ylcarbonyl, pyrrolidine-1-ylcarbonyl, etc.), carbamoyl, thiocarbamoyl, mono-C₁₋₆ alkyl-carbamoyl (e.g. methylcarbamoyl, ethylcarbamoyl, etc.), di-C₁₋₆ alkyl-carbamoyl (e.g. dimethylcarbamoyl, diethylcarbamoyl, ethylmethylcarbamoyl, etc.), mono- or di-C₆₋₁₄ aryl-carbamoyl (e.g. phenylcarbamoyl, 1-naphthylcarbamoyl, 2-naphthylcarbamoyl, etc.), mono- or di-5- or 6-membered heterocyclic carbamoyl (e.g. 2-pyridylcarbamoyl, 3-pyridylcarbamoyl, 4-pyridylcarbamoyl, 2-thienylcarbamoyl, 3-thienylcarbamoyl, etc.), C₁₋₆ alkylsulfonyl (e.g. methylsulfonyl, ethylsulfonyl, etc.), C₁₋₆ alkylsulfinyl (e.g. methylsulfinyl, ethylsulfinyl, etc.), C₆₋₁₄ arylsulfonyl (e.g. phenylsulfonyl, 1-naphthylsulfonyl, 2-naphthylsulfonyl, etc.), C₆₋₁₄ arylsulfinyl (e.g. phenylsulfinyl, 1-naphthylsulfinyl, 2-naphthylsulfinyl, etc.), formylamino, C₁₋₆ alkyl-carbonylamino (e.g. acetylamino etc.), C₆₋₁₄ aryl-carbonylamino (e.g. benzoylamino, naphthoylamino, etc.), C₁₋₆ alkoxy-carbonylamino (e.g. methoxycarbonylamino, ethoxycarbonylamino, propoxycarbonylamino, butoxycarbonylamino, etc.), C₁₋₆ alkylsulfonylamino (e.g. methylsulfonylamino, ethylsulfonylamino, etc.), C₆₋₁₄ arylsulfonylamino (e.g. phenylsulfonylamino, 2-naphthylsulfonylamino, 1-naphthylsulfonylamino, etc.), C₁₆-alkyl-carbonyloxy (e.g. acetoxy, propionyloxy, etc.), C₆₋₁₄ aryl-carbonyloxy (e.g. benzoyloxy, naphthylcarbonyloxy, etc.), C₁₋₆ alkoxy-carbonyloxy (e.g. methoxycarbonyloxy, ethoxycarbonyloxy, propoxycarbonyloxy, butoxycarbonyloxy, etc.), mono-C₁₋₆ alkyl-carbamoyloxy (e.g. methylcarbamoyloxy, ethylcarbamoyloxy, etc.), di-C₁₋₆ alkyl-carbamoyloxy (e.g. dimethylcarbamoyloxy, diethylcarbamoyloxy, etc.), C₆₋₁₄ aryl-carbamoyloxy (e.g. phenylcarbamoyloxy, naphthylcarbamoyloxy, etc.), nicotinoyloxy, optionally substituted 5- to 7-membered saturated cyclic amino(the substituent is selected from halogen atom, optionally halogenated C₁₋₆ alkyl, optionally halogenated C₃₋₆ cycloalkyl, C₆₋₁₄ aryl and optionally halogenated C₁₋₈ alkoxy), a 5- to 10-membered aromatic heterocyclic group (e.g. 2-thienyl, 3-thienyl, 2-pyridyl, 3-pyridyl, 4-pyridyl, 2-quinolyl, 3-quinolyl, 4-quinolyl, 5-quinolyl, 8-quinolyl, 1-isoquinolyl, 3-isoquinolyl, 4-isquinolyl, 5-isoquinolyl, 1-indolyl, 2-indolyl, 3-indolyl, 2-benzothiazolyl, 2-benzo[b]thienyl, 3-benzo[b]thienyl, 2-benzo[b]furanyl, 3-benzo[b]furanyl, etc.), sulfo, and the like.

The C₁₋₆ alkyl group may have, for example, the 1 to 5, preferably 1 to 3 aforementioned substituents at any possible positions and, when the number of substituents is 2 or more, respective substituents may be the same or different.

As R¹, a cyano group is preferable.

R² and R⁴ are the same or different, and each represents a hydrogen atom, a C₁₋₆ alkyl group, a C₃₋₆ cycloalkyl group, a trifluoromethyl group, an amino-C₁₋₆ alkyl group, a mono- or di-substituted amino-C₁₋₆ alkyl group, an optionally halogenated C₁₋₆ alkyl group substituted with an optionally substituted hydroxyl group, a C₂₋₆ alkenyl group substituted with an optionally substituted hydroxyl group, a C₁₋₆ alkyl group substituted with an optionally substituted and optionally oxidized thiol group, an optionally substituted and optionally oxidized thiol group, a cyano group, an acyl group, an optionally substituted oxazolyl group or a 1,3-dioxolan-2-yl group.

Examples of the C₁₋₆ alkyl group in R² and R⁴ include the same group as the C₁₋₆ alkyl group in the above R¹.

Examples of the C₃₋₆ cycloalkyl group include cyclopropyl, cyclobutyl, cyclopentyl, cyclohexyl, etc.

Examples of the “C₁₋₆ alkyl group” in the “amino-C₁₋₆ alkyl group” and the “mono- or di-substituted amino-C₁₋₆ alkyl group” include the same groups as those exemplified for the C₁₋₆ alkyl group in the above R¹.

Examples of the substituent of the “optionally substituted hydroxyl group” in the “optionally halogenated C₁₋₆ alkyl group substituted with an optionally substituted hydroxyl group” include a C₁₋₆ alkyl group (e.g. methyl, ethyl, propyl, isopropyl, butyl, isobutyl, sec-butyl, tert-butyl, pentyl, hexyl, etc.), a C₂₋₆ alkenyl group (e.g. vinyl, allyl, isopropenyl, 1-butenyl, 2-butenyl, 3-butenyl, 2-methyl-2-propenyl, 1-methyl-2-propenyl, 2-methyl-1-propenyl, etc.), a C₂₋₆ alkynyl group (e.g. ethynyl, propargyl, 1-butynyl, 2-butynyl, 3-butynyl, 1-hexynyl, etc.), a C₃₋₆ cycloalkyl group (e.g. cyclopropyl, cyclobutyl, cyclopentyl, cyclohexyl, etc.), a C₆₋₁₄ aryl group (e.g. phenyl, 1-naphthyl, 2-naphthyl, 2-biphenylyl, 3-biphenylyl, 4-biphenylyl, 2-anthryl, etc.), a C₇₋₁₆ aralkyl group (e.g. benzyl, phenethyl, diphenylmethyl, 1-naphthylmethyl, 2-naphthylmethyl, 2,2-diphenylethyl, 3-phenylpropyl, 4-phenylbutyl, 5-phenylpentyl, etc.), and the like. These substituents may be further substituted with the same substituent as that exemplified for the substituent of the “optionally substituted C₁₋₆ alkyl group” represented by the above R^{A}. In addition, examples of the “optionally halogenated C₁₋₆ alkyl group” include a C₁₋₆ alkyl group (e.g. methyl, ethyl, propyl, isopropyl, butyl, isobutyl, sec-butyl, tert-butyl, pentyl, hexyl, etc.) optionally having 1 to 5, preferably 1 to 3 halogen atoms (e.g. fluorine, chlorine, bromine, iodine, etc.), specifically, methyl, chloromethyl, difluoromethyl, trichloromethyl, trifluoromethyl, ethyl, 2-bromoethyl, 2,2,2-trifluoroethyl, pentafluoroethyl, propyl, 3,3,3-trifluoropropyl, isopropyl, butyl, 4,4,4-trifluorobutyl, isobutyl, sec-butyl, tert-butyl, pentyl, isopentyl, neopentyl, 5,5,5-trifluoropentyl, hexyl, 6,6,6-trifluorohexyl, etc.

Examples of the substituent of the “optionally substituted hydroxyl group” in the “C₂₋₆ alkenyl group substituted with an optionally substituted hydroxyl group” include the same substituents as those described above, and examples of the “C₂₋₆ alkenyl group” include vinyl, allyl, isopropenyl, 1-butenyl, 2-butenyl, 3-butenyl, 2-methyl-2-propenyl, 1-methyl-2-propenyl, 2-methyl-1-propenyl, etc.

Examples of the “optionally substituted and optionally oxidized thiol group” in the “C₁₋₆ alkyl group substituted with an optionally substituted and optionally oxidized thiol group” include a group represented by the formula S(O)ₙR⁶ (wherein R⁶ represents a hydrogen atom or the same group as that exemplified for the substituent of the “optionally substituted hydroxyl group”, and n represents 0, 1 or 2), and examples of the “C₁₋₆ alkyl group” include the same groups as those described above.

Examples of the “optionally substituted and optionally oxidized thiol group” include a group represented by the formula: —S(O)ₙ R⁶ (wherein R⁶ represents a hydrogen atom or the same group as that exemplified for the substituent of the “optionally substituted hydroxyl group”, and n represents 0, 1 or 2).

Examples of the “acyl group” include a group represented by the formula: —COOR⁷, —CONR⁸R⁹, —COR¹⁰ or —(C═S)—NR¹¹R¹² (wherein R⁷, R⁸, R⁹, R¹⁰, R¹¹ and R¹² are as defined with respect to the above R⁶, and R⁸ and R⁹, and R¹¹ and R¹² each may be taken together with the adjacent nitrogen atom to form an optionally substituted cyclic group optionally (the substituent: the same group as the substituent of the “optionally substituted hydroxyl group”) (e.g. a C₃₋₆ cycloalkyl group, a 5- or 6-membered heterocyclic group containing 1 to 3 heteroatoms selected from a nitrogen atom, a sulfur atom and an oxygen atom in addition to carbon atoms)). Specific examples include a lower (C₁₋₆)alkanoyl group (e.g. formyl, acetyl, propionyl, butyryl, isobutyryl, valeryl, isovaleryl, pivaloyl, hexanoyl, etc.), a lower (C₃₋₇)alkenoyl group (e.g. acryloyl, methacryloyl, crotonoyl, isocrotonoyl, etc.), a C₄₋₇ cycloalkanecarbonyl group (e.g. a cyclopropanecarbonyl group, a cyclobutanecarbonyl group, a cyclopentanecarbonyl group, a cyclohexanecabonyl group, etc.), a C₇₋₁₄ aroyl group (e.g. benzoyl, p-toluoyl, 1-naphthoyl, 2-naphthoyl, etc.), a C₆₋₁₀ aryl lower (C₂₋₄)alkanoyl group (e.g. phenylacetyl, phenylpropionyl, hydroatropoyl, phenylbutyryl, etc.), a C₆₋₁₀ aryl lower (C₃₋₅) alkenoyl group (e.g. cinnamoyl, atropoyl, etc.), and the like.

Examples of the substituent of the “optionally substituted oxazoyl group” include the same substituents as those exemplified for the substituent of the “optionally substituted C₁₋₆ alkyl group” represented by the above R^{A}, the oxazolyl group may have 1 to 5, preferably 1 to 3 substituents at any possible positions and, when the number of substituents is 2 or more, respective substituents may be the same or different.

As R², a C₁₋₆ alkyl group, a C₃₋₆ cycloalkyl group, a trifluoromethyl group, an amino-C₁₋₆ alkyl group, a mono- or di-substituted amino-C₁₋₆ alkyl group, a C₁₋₆ alkyl group substituted with an optionally substituted hydroxyl group, a C₁₋₆ alkyl group substituted with an optionally substituted and optionally oxidized thiol group, an optionally substituted and an optionally oxidized thiol group, a cyano group or an acyl group (inter alia, a C₁₋₆ alkyl group, a C₃₋₆ cycloalkyl group, a trifluoromethyl group, an amino-C₁₋₆ alkyl group, a mono- or di-substituted amino-C₁₋₆ alkyl group, a C₁₋₆ alkyl group substituted with an optionally substituted hydroxyl group, a C₁₋₆ alkyl group substituted with an optionally substituted and optionally oxidized thiol group, a cyano group or an acyl group) is preferable and, particularly, methyl, ethyl, propyl, isopropyl, cyclopropyl, trifluoromethyl, mono- or di-substituted amino-methyl, hydroxymethyl, hydroxyethyl, hydroxyisopropyl, C₁₋₆ alkyloxymethyl, C₁₋₆ alkylthio, C₁₋₆ alkylsulfonyl, C₁₋₆ alkylthiomethyl, C₁₋₆ alkylsulfonylmethyl; acetyl, carbamoyl or mono- or di-substituted carbamoyl (inter alia, methyl, ethyl, propyl, isopropyl, cyclopropyl, trifluoromethyl, mono- or di-substituted amino-methyl, hydroxymethyl, hydroxyethyl, hydroxyisopropyl, C₁₋₆ alkyloxymethyl, C₁₋₆ alkylthiomethyl, C₁₋₆ alkylsulfonylmethyl, acetyl, carbamoyl or mono- or di-substituted carbamoyl) is preferable.

As R⁴, methyl, ethyl, propyl, isopropyl, cyclopropyl, trifluoromethyl, mono- or di-substituted amino-methyl, hydroxymethyl, hydroxyethyl, hydroxyisopropyl, C₁₋₆ alkyloxymethyl, C₁₋₆ alkylthio, C₁₋₆ alkylsulfonyl, C₁₋₆ alkylthiomethyl, C₁₋₆ alkylsulfonylmethyl, acetyl, carbamoyl or mono- or di-substituted carbamoyl is preferable.

R³ represents a group represented by the above formula (a), preferably the formula (a′).

Among these formulas, Y represents a carbon atom or a nitrogen atom.

Alk represents an optionally substituted C₁₋₄ alkylene group, examples of the “C₁₋₄ alkylene group” include a straight chain C₁₋₄ alkylene group such as methylene, ethylene, propylene, butylene, etc., and examples of the “substituent” include the same group as the “substituent” of the C₁₋₆ alkyl group represented by the above R^{A}. Alk may have 1 to 4, preferably 1 to 2 substituents at any possible positions and, when the number of substituents is 2 or more, respective substituents may be the same or different.

As Alk, unsubstituted methylene is preferable.

X represents a halogen atom such as fluorine, chlorine, bromine, iodine, etc., preferably, chlorine or fluorine, particularly chlorine.

R^{B} represents a hydrogen atom or an acyl group, and examples of the acyl group include the same groups as those described above.

As R⁸, a hydrogen atom, succinoyl, dimethylaminomethylcarbonyl, etc. are preferable.

R⁵ represents a phenyl group which has a cyano group at a 4-position or a 3-position thereof, and may be further substituted.

Examples of the further substituent of the phenyl group include the same group as the “substituent” of the C₁₋₆ alkyl group represented by the above R^{A}

As R⁵, 4-cyanophenyl, 3-cyanophenyl, 4-cyano-3-(trifluoromethyl)phenyl, etc. are preferable.

Among them, as the compound (I) of the present invention, for example, a compound in which

R¹ is a hydrogen atom, a cyano group or a C₁₋₆ alkoxycarbonyl group,

R² is (i) a hydrogen atom,

(ii) an optionally halogenated C₁₋₆ alkyl group,

(iii) a C₃₋₆ cycloalkyl group,

(iv) a trifluoromethyl group,

(v) a C₁₋₆ alkyl group substituted with an amino mono- or di-substituted with C₁₋₆ alkyl,

(vi) an optionally halogenated C₁₋₆ alkyl group substituted with a hydroxyl group optionally substituted with C₁₋₆ alkyl,

(vii) a C₂₋₆ alkenyl group substituted with a hydroxyl group,

(viii) a C₁₋₆ alkyl group substituted with an optionally oxidized thiol group optionally substituted with C₁₋₆ alkyl,

(ix), an optionally oxidized thiol group optionally substituted with C₁₋₆ alkyl,

(x) a cyano group,

(xi) a C₁₋₆ alkanoyl group,

(xii) an oxazolyl group, or

(xiii) a 1,3-dioxolan-2-yl group,

Alk is a C₁₋₄ alkylene group optionally substituted with optionally halogenated C₁₋₆ alkyl,

R^{B} is (i) a hydrogen atom, (ii) a carbamoyl group, (iii) a carboxy-C₁₋₆ alkyl-carbonyl group or (iv) a mono- or di-C₁₋₆ alkylamino-C₁₋₆ alkyl-carbonyl group,

X is a halogen atom,

Y is a carbon atom or a nitrogen atom,

R⁴ is a C₁₋₆ alkyl group optionally substituted with a hydroxyl group, a trifluoromethyl group or a cyano group, and

R⁵ is a phenyl which has a cyano group at a 4-position or a 3-position thereof and may be further substituted with optionally halogenated C₁₋₆ alkyl is preferable.

Particularly, the compound (I) in which R¹ is a hydrogen atom, a cyano group or a methoxycarbonyl group, R² is a methyl group, an ethyl group, a propyl group, an isopropyl group, a cyclopropyl group, a trifluoromethyl group, a 1-hydroxyethyl group or an acetyl group, R³ is a 6-chloro-5-(hydroxymethyl)pyridin-3-ylmethyl group, a 6-chloro-5-(1-hydroxy-2,2,2-trifluoroethyl)pyridin-3-ylmethyl group, a 4-chloro-3-(1-hydroxy-2,2,2-trifluoroethyl)benzyl group, a 4-chloro-3-(acetoxymethyl)benzyl group or a 4-chloro-3-(hydroxymethyl)benzyl group, R⁴ is a cyano group, a methyl group or an ethyl group, and R⁵ is a 4-cyano-2-fluorophenyl group or a 4-cyanophenyl group is preferable.

In particular, the compound (I) in which R¹ is a hydrogen atom, a cyano group or a methoxycarbonyl group, R² is a methyl group, an ethyl group, a propyl group, a cyclopropyl group or a trifluoromethyl group, R³ is a 6-chloro-5-(hydroxymethyl)pyridin-3-ylmethyl group, a 6-chloro-5-(1-hydroxy-2,2,2-trifluoroethyl)pyridin-3-ylmethyl group, a 4-chloro-3-(1-hydroxy-2,2,2-trifluoroethyl)benzyl group, a 4-chloro-3-(acetoxymethyl)benzyl group or a 4-chloro-3-(hydroxymethyl)benzyl group, R⁴ is a cyano group, a methyl group or an ethyl group, and R⁵ is a 4-cyano-2-fluorophenyl group or a 4-cyanophenyl group is preferable.

Examples of the salt of the compound (I) of the present invention include a metal salt, an ammonium salt, a salt with an organic base, a salt with an inorganic acid, a salt with an organic acid, a salt with a basic or acidic amino acid, and the like. Preferable examples of the metal salt include an alkali metal salt such as a sodium salt, a potassium salt, and the like; an alkaline earth metal salt such as a calcium salt, a magnesium salt, a barium salt, and the like; an aluminum salt; and the like. Preferable examples of the salt with an organic base include salts with trimethylamine, triethylamine, pyridine, picoline, 2,6-lutidine, ethanolamine, diethanolamine, triethanolamine, cyclohexylamine, dicyclohexylamine, N,N′-dibenzylethylenediamine, and the like. Preferable examples of the salt with an inorganic acid include salts with hydrochloric acid, hydrobromic acid, nitric acid, sulfuric acid, phosphoric acid, and the like. Preferable examples of the salt with an organic acid include salts with formic acid, acetic acid, trifluoroacetic acid, phthalic acid, fumaric acid, oxalic acid, tartaric acid, maleic acid, citric acid, succinic acid, malic acid, methanesulfonic acid, benzenesulfonic acid, p-toluenesulfonic acid, and the like. Preferable examples of the salt with a basic amino acid include salts with arginine, lysine, ornithine, and the like, and preferable examples of the salt with an acidic amino acid include salts with aspartic acid, glutamic acid, and the like.

Among them, a pharmaceutically acceptable salt is preferable. When the compound has an acidic functional group, examples of such a salt include inorganic salts such as an alkali metal salt (e.g. a sodium salt, a potassium salt, etc.), an alkaline earth metal salt (e.g. a calcium salt, a magnesium salt, a barium salt, etc.), and the like, an ammonium salt, and the like and, when the compound has a basic functional group, examples of such a salt include salts with an inorganic acid such as hydrochloric acid, hydrobromic acid, nitric acid, sulfuric acid, phosphoric acid, and the like, and salts with an organic acid such as acetic acid, phthalic acid, fumaric acid, oxalic acid, tartaric acid, maleic acid, citric acid, succinic acid, methanesulfonic acid, p-toluenesulfonic acid, and the like.

The prodrug of the compound (I) of the present invention refers to a compound which is converted into the compound (I) by a reaction with an enzyme or gastric acid under the physiological conditions in a living body, that is, a compound which is converted into the compound (I) by enzymatic oxidation, reduction or hydrolysis, or a compound which is converted into the compound (I) by hydrolysis with gastric acid. Examples of the prodrug of the compound (I) include a compound in which an amino group of the compound (I) is acylated, alkylated or phosphorylated (e.g. a compound in which an amino group of the compound (I) is eicosanoylated, alanylated, pentylaminocarbonylated, (5-methyl-2-oxo-1,3-dioxolen-4-yl)methoxycarbonylated, tetrahydrofuranylated, pyrrolidinylmethylated, pivaloyloxymethylated, tert-butylated, etc.); a compound in which a hydroxyl group of the compound (I) is acylated, alkylated, phosphorylated or subjected to borate formation (e.g. a compound in which a hydroxyl group of the compound (I) is acetylated, palmitoylated, propanoylated, pivaloylated, succinylated, fumarylated, alanylated, or dimethylaminomethylcarbonylated, etc.); a compound in which a carboxyl group of the compound (I) is esterified, or amidated (e.g. a compound in which a carboxyl group of the compound (I) is ethyl esterified, phenyl esterified, carboxymethyl esterified, dimethylamino-methyl esterified, pivaloyloxymethyl esterified, ethoxycarbonyloxyethyl esterified, phthalidyl esterified, (5-methyl-2-oxo-1,3-dioxolen-4-yl)methyl esterified, cyclohexyloxycarbonylethyl esterified, methylamidated, etc.); and the like. These compounds can be prepared from the compound (I) by a known per se method.

Alternatively, the prodrug of the compound (I) may be a compound which is converted into the compound (I) under the physiological conditions as described in “Pharmaceutical Research and Development”, Vol. 7 (Drug Design), pages 163-198 published in 1990 by Hirokawa Publishing Co. (Tokyo, Japan).

As the compound of the present invention or a salt or prodrug thereof, for example,

(i) 1-{[6-chloro-5-(hydroxymethyl)pyridin-3-yl]methyl}-4-(4-cyanophenyl)-2,5-dimethyl-1H-pyrrole-3-carbonitrile,

(ii) 1-{[6-chloro-5-(hydroxymethyl)pyridin-3-yl]methyl}-4-(4-cyanophenyl)-2-ethyl-5-methyl-1H-pyrrole-3-carbonitrile,

(iii) 1-{[6-chloro-5-(hydroxymethyl)pyridin-3-yl]methyl}-4-(4-cyanophenyl)-5-methyl-2-propyl-1H-pyrrole-3-carbonitrile,

(iv) 1-{[6-chloro-5-(hydroxymethyl)pyridin-3-yl]methyl}-4-(4-cyanophenyl)-2-cyclopropyl-5-methyl-1H-pyrrole-3-carbonitrile,

(v) 1-{[6-chloro-5-(hydroxymethyl)pyridin-3-yl]methyl}-4-(3-cyanophenyl)-2,5-dimethyl-1H-pyrrole-3-carbonitrile,

(vi) 1-[4-chloro-3-(hydroxymethyl)benzyl]-4-(4-cyanophenyl)-2,5-dimethyl-1H-pyrrole-3-carbonitrile,

(vii) 2-chloro-5-{[3-cyano-4-(4-cyanophenyl)-2,5-dimethyl-1H-pyrrol-1-yl]methyl}benzyl N,N-dimethylglycinate,

(viii) 4-[(2-chloro-5-{[3-cyano-4-(4-cyanophenyl)-2,5-dimethyl-1H-pyrrol-1-yl]methyl}benzyl)oxy]-4-oxobutanoic acid,

(ix) 4-[(2-chloro-5-{[3-cyano-4-(4-cyanophenyl)-5-methyl-2-propyl-1H-pyrrol-1-yl]methyl}pyridin-3-yl)methoxy]-4-oxobutanoic acid,

(x) 4-[(2-chloro-5-{[3-cyano-4-(4-cyanophenyl)-2-ethyl-5-methyl-1H-pyrrol-1-yl]methyl}pyridin-3-yl)methoxy]-4-oxobutanoic acid,

(xi) 4-[(2-chloro-5-{[3-cyano-4-(4-cyanophenyl)-2-cyclopropyl-5-methyl-1H-pyrrol-1-yl]methyl}pyridin-3-yl)methoxy]-4-oxobutanoic acid,

(xii) 1-{[6-chloro-5-(2,2,2-trifluoro-1-hydroxyethyl)pyridin-3-yl]methyl}-4-(4-cyanophenyl)-2,5-dimethyl-1H-pyrrole-3-carbonitrile,

(xiii) 4-(1-{[6-chloro-5-(hydroxymethyl)pyridin-3-yl]methyl}-2,5-dimethyl-1H-pyrrol-3-yl)benzonitrile,

(xiv) 4-(1-{[6-chloro-5-(hydroxymethyl)pyridin-3-yl]methyl}-5-cyclopropyl-2-methyl-1H-pyrrol-3-yl)benzonitrile, or

(xv) 1-{[6-chloro-5-(hydroxymethyl)pyridin-3-yl]methyl}-3-(4-cyanophenyl)-5-methyl-1H-pyrrole-2-carbonitrile, or a salt thereof, etc. is preferable.

The compound (I) of the present invention or a salt thereof is obtained by a method shown by the following reaction scheme 1 or a similar method, or a known method or a similar method.

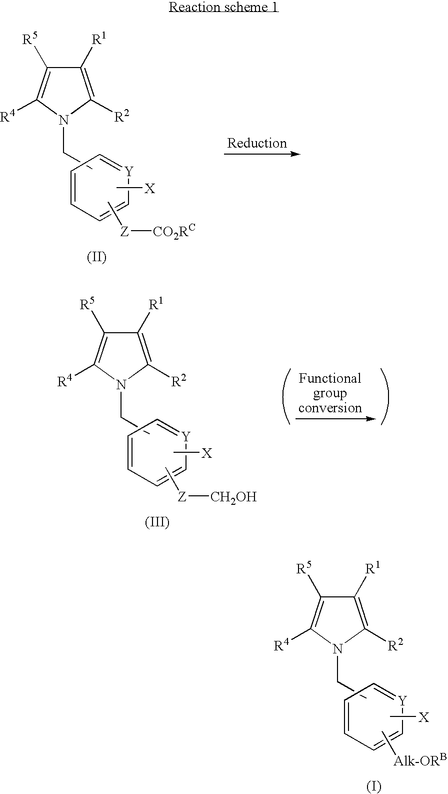

In the reaction scheme 1, Z represents a bond or an optionally substituted C₁₋₃ alkylene group, R^{C} represents an optionally substituted C₁₋₆ alkyl group, the other symbols are as defined above and, if necessary, may be protected with a protecting group which is generally used in an organic synthesis. The compounds in the reaction scheme include their salts, and examples of the salts include the same salts as those of the compound (I).

Examples of the “C₁₋₃ alkylene group” of the “optionally substituted C₁₋₃ alkylene group” represented by Z include a straight chain C₁₋₃ alkylene group such as methylene, ethylene, propylene, etc., and examples of the “substituent” include the same group as the “substituent” of the C₁₋₆ alkyl group represented by the above R^{A}.

As the “optionally substituted C₁₋₆ alkyl group” represented by R^{C}, the same group as the “optionally substituted C₁₋₆ alkyl group” represented by R^{A} is used.

The compound (II) used as a starting material can be synthesized by a known method (e.g. JP 2003-252854 A) or a similar method and, for example, can be prepared by a method shown in Reference Example hereinafter. A compound represented by the formula (III) itself is included in the scope of the compound represented by the formula (I), or can be led into a compound included in the formula (I) by converting a functional group thereof by a known per se method or a similar method (e.g. oxidizing reaction, reducing reaction, hydrolyzing reaction, acylating reaction, alkylating reaction, amidating reaction, aminating reaction, rearrangement reaction, etc.).

In the reducing reaction in the reaction scheme 1, a reducing agent which is generally used in organic synthesis such as sodium borohydride, sodium cyanoborohydride, sodium triacetoxyborohydride, lithium borohydride, and aluminum lithium hydride, etc. is used and, further, a metal salt such as calcium chloride, etc. may be added. An amount of the reducing agent to be used is about 1.0 to about 10 mole, preferably about 1.0 to 5.0 mole relative to 1 mole of the compound (II).

The reaction temperature is about −70° C. to about 100° C., preferably about 0° C. to about 50° C. The reaction time is around about 30 minutes to about 20 hours.

This reaction is usually performed in an organic solvent having no influence on the reaction. Examples of the organic solvent having no adverse influence on the reaction include alcohols such as methanol, ethanol, 2-propanol, etc., ethers such as diethyl ether, dioxane, tetrahydrofuran (THF), etc., saturated hydrocarbons such as hexane, pentane, etc., aromatic hydrocarbons such as benzene, toluene, etc., and the like. They may be used alone, or by mixing two or more kinds thereof at an appropriate ratio.

The acylating reaction for converting a functional group can be preformed by a method generally employed in organic synthesis, for example, by reacting the compound (III) with an acylating agent such as an organic acid, an acyl halide, an acid anhydride, etc. in a solvent having no influence on the reaction, if necessary, in the presence of a base. Examples of the solvent include ethers, hydrocarbons, halogenated hydrocarbons, ketones, nitrites, amides, esters, aromatic amines, heterocycles, etc. The preferred solvents include ethers, hydrocarbons, halogenated hydrocarbons, amides, aromatic amines and heterocycles. These solvents may be used alone, or by mixing two or more kinds thereof at an appropriate ratio.

Examples of the base include alkali metal salts such as sodium hydride, potassium carbonate, sodium carbonate, potassium hydroxide, sodium hydroxide, etc., and organic bases such as triethylamine, diisopropylethylamine, pyridine, etc.

The amount of the acylating agent to be used is usually 1 to 20 mole equivalents, preferably 2 to 10 mole equivalents relative to 1 mole of the compound (III). The amount of the base to be used is usually 1 to 10 mole equivalents, preferably 1 to 5 mole equivalents relative to 1 mole of the compound (III). Further, an excess amount of the base can be used as a solvent.

The oxidizing reaction for converting a functional group can be performed by using an oxidizing agent generally employed in organic synthesis, for example, a manganese compound such as potassium permanganate, manganese dioxide, etc., a chromium compound such as chromic acid, etc., or a sulfur compound such as dimethylsulfoxide, etc. in a solvent having no influence on the reaction, if necessary, in the presence of an acid, a base, etc. Examples of the solvent include water, hydrocarbons, halogenated hydrocarbons, alcohols, ketones, organic acids, amides, esters, solfoxides, etc. These solvents may be used alone, or by mixing two or more kinds thereof at an appropriate ratio.

Examples of the acid include mineral acids such as sulfuric acid, etc., organic acids such as acetic acid, etc., and the like. Examples of the base include alkali metal salts such as potassium hydroxide, sodium hydroxide, etc. and amines such as triethylamine, diisopropylethylamine, piperidine, etc. In addition, if necessary, a dehydrating agent such as dicyclohexylcarbodiimide, etc., oxalyl chloride, pyridine, sulfur trioxide and the like can be added.

The amount of the oxidizing agent to be used is usually 1 to 20 mole eequivalents, preferably 1 to 10 mole equivalents relative to 1 mole of the compound (III). Further, an excess amount of the oxidizing agent can be used as a solvent. The amount of the acid or base to be used is usually 1 to 20 mole equivalents, preferably 1 to 10 mole equivalent relative to 1 mole of the compound (III). The amount of other additives to be used is usually 1 to 20 mole equivalents, preferably 1 to 10 mole equivalents relative to 1 mole of the compound (III).

The reaction temperature is usually −70 to 120° C., preferably −70 to 100° C. The reaction time is usually, 0.1 to 100 hours, preferably 0.1 to 48 hours.

The alkylating reaction of a carbonyl compound, for example, that obtained by oxidation of the compound (III) can be preformed by reacting the carbonyl compound with an alkylating agent in a solvent having no influence on the reaction, if necessary, in the presence of an additive.

Examples of the alkylating agent include an organic magnesium reagent such as an alkyl magnesium halide, etc., an organic silicon compound such as (trifluoromethyl)trimethylsilane, etc., and the like.

Examples of the solvent include hydrocarbons, ethers, etc. These solvent may be used alone, or by mixing two or more kinds thereof at an appropriate ratio.

The reaction using an organic silicon compound can be performed according to a per se known method (e.g. the method described in Journal of American Chemical Society (1989), pp. 393-395), or a similar method. Since an androgen receptor antagonist including the compound (I) of the present invention or a salt thereof (hereinafter, sometimes, abbreviated as the present androgen receptor antagonist) has excellent androgen receptor antagonism, has low toxicity and little side effects, it is useful as a safe medicament, or an androgen receptor antagonist.

Since a pharmaceutical composition containing the present androgen receptor antagonist exhibits excellent androgen receptor antagonism and/or prostate-specific antigen (PSA) production inhibitory activity for a mammal (e.g. mouse, rat, hamster, rabbit, cat, dog, bovine, sheep, monkey, human, etc.), and excellent in (oral) absorbability and (metabolism) stability, it can be used as an agent for preventing or treating androgen receptor-associated diseases, for example, hormone-sensitive diseases at an androgen dependent term and/or at an androgen independent term, particularly, hormone-sensitive cancers at an androgen dependent term and/or at an androgen independent term (e.g. prostate cancer, uterus cancer, breast cancer, pituitary gland cancer, liver cancer, etc.), and sexual hormone-sensitive diseases such as prostatic hypertrophy, endometriosis, precocious puberty, dysmenorrhea, amenorrhea, premenstrual syndrome, multilocular uterus syndrome, etc., as a contraceptive (or an agent for preventing or treating infertility when rebound effect after medication posing is used), and the like.

In particular, since the compound (I) of the present invention or a salt thereof exhibits antagonism for a normal androgen receptor and/or a mutated receptor, it can exert excellent preventing or treating effect for a hormone-sensitive cancer at an androgen dependent term and/or at an androgen independent term.

Among androgen receptor antagonists, a drug exhibiting antagonism for a mutated androgen receptor or a drug exhibiting antagonism for an androgen receptor having increased sensitivity is also useful as an agent for preventing or treating a hormone-sensitive cancer at an androgen dependent term and/or at an androgen independent term.

A pharmaceutical composition containing the androgen receptor antagonist of the present invention can be safely administered orally or parenterally (e.g. local, rectal, intravenous administration) as a pharmaceutical preparation such as a tablet (including sugar-coated tablet, and film coating tablet), a powder, a granule, a capsule (including soft capsule), a solution, an injectable preparation, a suppository, a sustained-release preparation, or the like obtained by mixing the androgen receptor antagonist of the present invention with a pharmacological acceptable carrier according to a known per se method. An injectable preparation can be administered intravenously, intramuscularly, subcutaneously or to an organ, or can be administered directly to a lesion.

Examples of the pharmacologically acceptable carrier which may be used in preparing the pharmaceutical composition of the present invention include various organic or inorganic carrier substances which are conventionally used as a preparation material, for example, an excipient, a lubricant, a binder and a disintegrating agent in a solid preparation, and a solvent, a solubilizer, a suspending agent, an isotonic, a buffer and a soothing agent in a liquid preparation. Further, if necessary, an additive such as a usual antiseptic, antioxidant, coloring agent, sweetener, adsorbing agent, wetting agent, etc. can be appropriately used at an appropriate amount.

Examples of then excipient include lactose, white sugar, D-mannitol, starch, corn starch, crystalline cellulose, light silicic acid anhydride, etc.

Examples of the lubricant include magnesium stearate, calcium stearate, talc, colloidal silica, etc.

Examples of the binder include crystalline cellulose, white sugar, D-mannitol, dextrin, hydroxypropylcellulose, hydroxypropylmethylcellulose, polyvinylpyrrolidone, starch, sucrose, gelatin, methylcellulose, sodium carboxymethylcellulose, etc.

Examples of the disintegrating agent include starch, carboxymethylcellulose, potassium carboxymethylcellulose, sodium carboxymethylstarch, L-hydroxypropylcellulose, etc.

Examples of the solvent include water for injection, alcohol, propylene glycol, macrogol, sesame oil, corn oil, olive oil, etc.

Examples of the solubilizer include polyethylene glycol, propylene glycol, D-mannitol, benzyl benzoate, ethanol, trisaminomethane, cholesterol, triethanolamine, sodium carbonate, sodium citrate, etc.

Examples of the suspending agent include surfactants such as stearyl triethanolamine, sodium laurylsulfate, laurylaminopropionate, lecithin, benzalkonium chloride, benzethonium chloride, glycerin monostearate, etc.; hydrophilic polymers such as polyvinyl alcohol, polyvinyl pyrrolidone, sodium carboxymethylcellulose, methylcellulose, hydroxymethylcellulose, hydroxyethylcellulose, hydroxypropylcellulose, etc.; and the like.

Examples of the isotonic include glucose, D-sorbitol, sodium chloride, glycerin, D-mannitol, etc.

Examples of the buffer include buffers such as phosphate, acetate, carbonate, citrate, etc.

Examples of the soothing agent include benzyl alcohol, etc.

Examples of the antiseptic include paraoxybenzoic acid esters, chlorobutanol, benzyl alcohol, phenethyl alcohol, dehydroacetic acid, sorbic acid, etc.

Examples of the antioxidant include sulfite, ascorbic acid, α-tocopherol, etc.

The content of the androgen receptor antagonist of the present invention in the pharmaceutical composition of the present invention can be appropriately selected depending on particular administration subject, administration route, disease, etc. For example, although the content is different depending on the dosage form of a preparation, it is usually about 0.01 to 100% by weight, preferably about 0.1 to 50% by weight, more preferably about 0.5 to 20% by weight relative to the total preparation.

The content of an additive such as a carrier in the pharmaceutical composition of the present invention is different depending on the dosage form of a preparation, and is usually about 1 to 99.99% by weight, preferably about 10 to 90% by weight relative to the total preparation.

Examples of a drug which can be used together with the androgen receptor antagonist of the present invention (hereinafter, abbreviated to combined use drug) include an LH-RH derivative.

Examples of the LH-RH derivative include an LH-RH derivative or a salt thereof useful in a hormone-dependent disease, particularly, sexual hormone-dependent cancer (e.g. prostate cancer, uterus cancer, breast cancer, pituitary gland cancer, liver cancer, etc.), and a sexual hormone-sensitive disease such as prostatic hypertrophy, endometriosis, precocious puberty, dysmenorrheal, amenorrhea, premenstrual syndrome, multilocular uterus syndrome, etc., and contraception (or infertility when rebound effect after medication posing thereof is utilized). Further, the examples include an LH-RH derivative or a salt thereof effective in benign or malignant tumor which is sexual hormone independent but is LH-RH-sensitive.

Specific examples of the LH-RH derivative or a salt thereof include peptides described in Treatment with GnRH analogs: Controversies and perspectives, published by The Parthenon Publishing Group Ltd. in 1996, JP 3-503165 A, JP 3-101695 A, JP 7-97334 A, JP 8-259460 A, etc.

As the LH-RH derivative, there are an LH-RH agonist and an LH-RH antagonist and, as the LH-RH antagonist, for example, a physiologically active peptide represented by the formula:
X-D2Nal-D4ClPhe-D3 Pal-Ser-A-B-Leu-C-Pro-DAlaNH₂
   wherein X represents N(4H₂-furoyl)Gly or NAc, A represents a residue selected from NMeTyr, Tyr, Aph(Atz) and NMeAph(Atz), B represents a residue selected from DLys(Nic), DCit, DLys(AzaglyNic), DLys(AzaglyFur), DhArg(Et₂), DAph(Atz) and DhCi, and C represents Lys(Nisp), Arg or hArg(Et₂), or a salt thereof is used.

As the LH-Rh agonist, for example, a physiologically active peptide represented by the formula:
5-oxo-Pro-His-Trp-Ser-Tyr-Y-Leu-Arg-Pro-Z
   wherein Y represents a residue selected from DLeu, DAla, DTrp, DSer(tBu), D2Nal and DHis(ImBzl), and Z represents NH—C₂H₅ or Gly-NH₂, or a salt thereof is used. In particular, a peptide in which Y is DLeu and Z is NH—C₂H₅ (i.e. peptide A represented by 5-oxo-Pro-His-Trp-Ser-Tyr-DLeu-Leu-Arg-Pro-NH—C₂H₅; leuproreline) or a salt thereof (e.g. acetate) is suitable.

A medicament comprising a combination of the present androgen receptor antagonist and the combined use drug (hereinafter, the present combination use preparation) has low toxicity and, for example, can be safely administered orally or parenterally (e.g. local, rectal, intravenous administration etc.) as a pharmaceutical preparation such as a tablet (including a sugar-coated tablet, and a film coating tablet), a powder, a granule, a capsule (including a soft capsule), a solution, an injectable preparation, a suppository, a sustained-release preparation, or the like by mixing the present androgen receptor antagonist and/or the combined use drug with a pharmacologically acceptable carrier according a known per se method. An injectable preparation can be administered intravenously, intramuscularly, subcutaneously or to an organ, or can be administered directly to a lesion.

Examples of the pharmacologically acceptable carrier which may be used in preparing the present combination use preparation include various organic and inorganic carrier substances which are conventionally used as a pharmaceutical material, such as an excipient, a lubricant, a binder and a disintegrating agent in a solid preparation, and a solvent, a solubilizer, a suspending agent, an isotonic, a buffer and a soothing agent in a liquid preparation. Further, if necessary, an additive such as a usual antiseptic, antioxidant, coloring agent, sweetener, adsorbing agent, and wetting agent may be appropriately used at an appropriate amount.

Examples of the excipient include lactose, white sugar, D-mannitol, starch, corn starch, crystalline cellulose, light silicic acid anhydride, etc.

Examples of the lubricant include magnesium stearate, calcium stearate, talc, colloidal silica, etc.

Examples of the binder include crystalline cellulose, white sugar, D-mannitol, dextrin, hydroxypropylcelluloce, hydroxypropylmethylcellulose, polyvinylpyrrolidone, starch, sugar, gelatin, methylcellulose, sodium carboxymethylcellulose, etc.

Examples of the disintegrating agent include starch, carboxymethylcellulose, calcium carboxymethylcellulose, sodium carboxymethylstarch, L-hydroxypropylcellulose, etc.

Examples of the solvent include water for injection, alcohol, propylene glycool, macrogol, sesame oil, corn oil, olive oil, etc.

Examples of the solubilizer include polyethylene glycol, propylene glycol, D-mannitol, vinyl benzoate, ethanol, trisaminomethane, cholesterol, triethanolamine, sodium carbonate, sodium citrate, etc.

Examples of the suspending agent include surfactants such as stearyltriethanolamine, sodium laurylsulfate, laurylaminopropionate, lecithin, benzalkonium chloride, benzethonium chloride, glycerin monostearate, etc.; hydrophilic polymers such as polyvinyl alcohol, polyvinyl pyrrolidone, sodium carboxymethylcellulose, methylcellulose, hydroxymethylcellulose, hydroxyethylcellulose, hydroxypropylcellulose, etc.; and the like.

Examples of the isotonic include glucose, D-sorbitol, sodium chloride, glycerin, D-mannitol, etc.

Examples of the buffer include buffers such as phosphate, acetate, carbonate, citrate, etc.

Examples of the soothing agent include benzyl alcohol, etc.

Examples of the antiseptic include paraoxybenzoic acid esters, chlorobutanol, benzyl alcohol, phenethyl alcohol, dehydroacetic acid, sorbic acid, etc.

Examples of the antioxidant include sulfite, ascorbic acid, α-tocopherol, etc.

The ratio of blending the present androgen receptor antagonist and the combined use drug in the present combination use preparation can be appropriately selected depending on particular administration subject, administration route, disease, and the like.

For example, the content of the present androgen receptor antagonist in the present combination use preparation is different depending on the dosage form of a preparation, and is usually about 0.01 to 100% by weight, preferably about 0.1 to 50% by weight, more preferably about 0.5 to 20% by weight relative to the total preparation.

The content of the combined use drug in the present combination use preparation is different depending on the dosage form of a preparation, and is usually about 0.01 to 100% by weight, preferably about 0.1 to 50% by weight, more preferably about 0.5 to 20% by weight.

The content of an additive such as a carrier in the present combination use preparation is different depending on the dosage form of a preparation, and is usually about 1 to 99.99% by weight, preferably about 10 to 90% by weight relative to the total preparation.

In addition, when the present androgen receptor antagonist and the combined use drug are formulated into separate preparations, the same contents can be used.

These preparations can be prepared by a known per se method which is usually used in pharmaceutical production steps.

For example, the present androgen receptor antagonist or the combined use drug can be formulated into an injectable preparation by using it together with a dispersant (e.g. Tween 80 (manufactured by Atlas Powder, USA), HCO 60 (manufactured by Nikko Chemicals Co., Ltd.), polyethylene glycol, carboxymethylcellulose, sodium alginate, hydroxypropylmethyllcellulose, dextrin, etc.), a stabilizer (e.g. ascorbic acid, sodium pyrosulfate, etc.), a surfactant (e.g. Polysorbate 80, macrogol, etc.), a solubilizer (e.g. glycerin, ethanol, etc.), a buffer (e.g. phosphoric acid and an alkali metal salt thereof, citric acid and an alkali metal salt thereof, etc.), an isotonic (e.g. sodium chloride, potassium chloride, mannitol, sorbitol, glucose, etc.), a pH adjusting agent (e.g. hydrochloric acid, sodium hydroxide, etc.), a preservative (e.g. ethyl paraoxybenzoate, benzoic acid, methylparaben, propylparaben, benzyl alcohol, etc.), a dissolving agent (e.g. concentrated glycerin, meglumine, etc.), a dissolution aid (e.g. propylene glycol, white sugar, etc.), a soothing agent (e.g. glucose, benzyl alcohol, etc.), etc. to obtain an aqueous injectable preparation, or by dissolving, suspending or emulsifying it in a vegetable oil such as olive oil, sesame oil, cottonseed oil, corn oil, etc., or a dissolution aid such as propylene glycol, etc. to obtain an oily injectable preparation.

An oral preparation can be prepared according to a know per se method by adding an excipient (e.g. lactose, white sugar, starch, etc.), a disintegrating agent (e.g. starch, calcium carbonate, etc.), a binder (e.g. starch, gum arabic, carboxymethylcellulose, polyvinylpyrrolidone, hydroxypropylcellulose, etc.) or a lubricant (e.g. talc, magnesium stearate, polyethylene glycol 6000, etc.) to the present androgen receptor antagonist or the combined use drug, compression-molding the resulting mixture and, if necessary, coating it by a method known per se for the purpose of taste masking, enteric property or sustaining property. Examples of the coating agent to be used include hydroxypropylmethylcellulose, ethylcellulose, hydroxymethylcellulose, hydroxypropylcellulose, polyoxyethylene glycol, Tween 80, Pluronic F68, cellulose acetate phthalate, hydroxypropylmethylcellulose phthalate, hydroxymethylcellulose acetate succinate, Eudragit (manufactured by Rohm, German, methacrylic acid acrylic copolymer), and a pigment (e.g. bengala, titanium dioxide, etc.). The oral preparation may be either of a rapid-releasing preparation or a sustained-releasing preparation.

For example, in order to obtain a suppository, the present androgen receptor antagonist or the combined use drug can be formulated into an aqueous solid, semisolid or liquid suppository according to a known per se method. Examples of the oily base to be used in the aforementioned composition include glycerides of higher fatty acids [e.g. cacao butter, witepsol (manufactured by Dynamite Novel, German), etc.], medium fatty acids [e.g. miglyol (manufactured by Dynamite Novel, German), etc.], vegetable oils (e.g. sesame oil, soybean oil, cottonseed oil, etc.), and the like. Examples of the aqueous base to be used include polyethylene glycols, and propylene glycol, and examples of the aqueous gel base include natural gums, cellulose derivatives, vinyl polymers, acrylic acid polymers, etc.

Examples of the sustained-release preparation include sustained-release microcapsule preparation.

For formulating into sustained-release microcapsule preparation, a known per se method can be adopted and, for example, it is preferable to administer the sustained-release preparation molded by the method shown in [2] hereinafter.

Preferably, the present androgen receptor antagonist is administered by molding it into an oral preparation such as a solid preparation (e.g. powder, granule, tablet, capsule, etc.), or molding it into a rectal preparation such as a suppository, etc. Particularly, an oral preparation is preferable.

The combined use drug can be formulated into the aforementioned dosage form depending on a particular kind of a drug.

Hereinafter, [1] an injectable preparation of the present androgen receptor antagonist or the combined use drug, and its production, [2] a sustained-release preparation or a rapid-release preparation of the present androgen receptor antagonist or the combined use drug, and its production, and [3] a sublingual tablet, a buccal or an oral cavity rapid-disintegrating agent of the present androgen receptor antagonist or the combined use drug, and its production will be specifically illustrated.

[1] Injectable Preparation and its Production

In a preferred injectable preparation, the present androgen receptor androgen or the combined use drug is dissolved in water. The injectable preparation may contain a benzoate and/or a salicylate.

The injectable preparation is obtained by dissolving the present androgen receptor antagonist or the combined use drug and, optionally, a benzoate and/or a salicylate in water.

Examples of a salt of the benzoic acid or salicylic acid include an alkali metal salt such as sodium, potassium, etc., an alkaline earth metal salt such as calcium, magnesium, etc., an ammonium salt, a meglumine salt, other organic acid salts such as trometamol, etc., and the like.

The concentration of the present androgen receptor antagonist or the combined use drug in the injectable is 0.5 to 50 w/v %, preferably around 3 to 20 w/v %. Further, the concentration of a benzoate and/or a salicylate is 0.5 to 50 w/v %, preferably 3 to 20 w/v %.

In addition, an additive such as a stabilizer (ascorbic acid, sodium pyrosulfite, etc.), a surfactant (Polysorbate 80, macrogol, etc.), a solubilizer (glycerin, ethanol, etc.), a buffer (phosphoric acid and an alkali metal salt thereof, citric acid and an alkali metal salt thereof, etc.), an isotonic (sodium chloride, potassium chloride, etc.), a dispersant (hydroxypropylmethylcellulose, dextrin, etc.), a pH adjusting agent (hydrochloric acid, sodium hydroxide, etc.), a preservative (ethyl paraoxybenzoate, benzoic acid, etc.), a dissolving agent (concentrated glycerin, meglumine, etc.), a dissolution aid (propylene glycol, white sugar, etc.), a soothing agent (glucose, benzyl alcohol, etc.), or the like which is generally used in an injectable preparation can be appropriately blended into the present preparation. These additives are generally blended at a ratio which is conventionally used in an injectable preparation.

Preferably, the injectable preparation is adjusted to a pH of 2 to 12, preferably 2.5 to 8.0 by adding a pH adjusting agent.

The injectable preparation is obtained by dissolving the present androgen receptor antagonist or the combined use drug and, optionally, a benzoate and/or a salicylate and, if necessary, the additive in water. Dissolution of them may be performed in an arbitrary order, and can be appropriately performed according to a conventional production process of an injectable preparation.

An aqueous solution for injection is preferably warmed and, as in a conventional injectable preparation, it is sterilized, for example, by filtration or heating under high pressure to obtain an injectable preparation.

Preferably, an aqueous solution for injection is sterilized by heating at 100° C. to 121° C. for 5 minutes to 30 minutes under high pressure.

Further, for use as a multiple-divided administration preparation, a preparation to which antibacterial property of a solution is imparted can be produced.

[2] Sustained-Release Preparation or Rapid-Release Preparation and its Production

A sustained-release preparation in which a core containing the present androgen receptor antagonist or the combined use drug is optionally covered with a covering agent such as a water-insoluble substance, a swelling polymer, etc. is preferable. For example, a single dose per day-type oral sustained-release preparation is preferable.

Examples of the water-insoluble substance used in a covering agent include cellulose ethers such as ethylcellulose, butylcellulose, etc., cellulose esters such as cellulose stearate, cellulose propionate, etc., polyvinyl esters such as polyvinyl acetate, polyvinyl butyrate, etc., acrylic acid-based polymers such as an acrylic acid/methacrylic acid copolymer, a methyl methacrylate copolymer, an ethoxyethyl methacrylate/cinnamonethyl methacrylate/aminoalkyl methacrylate copolymer, polyacryilic acid, polymethacrylic acid, a methacrylic acid alkylamide copolymer, poly(methyl methacrylate), polymethacrylate, polymethacrylamide, an aminoalkyl methacrylate copolymer, poly(methacrylic acid anhydride), a glycidyl methacrylate copolymer, inter alia, Eudragits (Rohm Pharma) such as Eudragit RS-100, RL-100, RS-30D, RL-30D, RL-PO, RS-PO (ethyl acrylate/methyl methacrylate/trimethyl methacrylate chloride/ammonium ethyl copolymer), Eudragit NE-30D (methyl methacrylate/ethyl acrylate copolymer), etc., hydrogenated oils such as hydrogenated castor oil (e.g. Lovely Wax (Freund Industry) etc.), etc., waxes such as carnauba wax, fatty acid glycerin esters, paraffin, etc., polyglycerin fatty acid esters, and the like.

As the swelling polymer, a polymer which has an acidic dissociating group and exhibits pH dependent swelling is preferable, and a polymer which is slightly swollen in an acidic region such as in stomach, and has such an acidic dissociating group that swelling becomes great in a neutral region such as small intestine and large intestine is preferable.

Examples of such the polymer which has an acidic dissociating group and exhibits pH dependent swelling include crosslinked polyacrylic acid polymers such as Carbomer 934P, 940, 941, 974P, 980 and 1342, polycarbophil, and calcium polycarbophil (all manufactured by BF Goodrich), Hiviswako 103, 104, 105 and 304 (all manufactured by Wako Pure Chemical Industries, Ltd.), etc.

The covering agent used in a sustained-release preparation may further contain a hydrophilic substance.

Examples of the hydrophilic substance include polysaccharides optionally having a sulfate group such as pullulan, dextrin, alginic acid, etc., alkali metal salt, polysaccharides having a hydroxyalkyl group or a carboxyalkyl group such as hydroxypropylcellulose, hydroxypropylmethylcellulose, sodium carboxymethylcellulose, methylcellulose, etc., polyvinyl pyrrolidone, polyvinyl alcohol, polyethylene glycol, etc.

The content of the water-insoluble substance in the covering agent of the sustained-release preparation is about 30 to about 90% (w/w), preferably about 35 to about 80% (w/w), more preferably about 40 to 75% (w/w/), and the content of the swelling polymer is about 3 to about 30% (w/w), preferably about 3 to about 15% (w/w). The covering agent may further contain the hydrophilic substance and, in that case, the content of the hydrophilic substance in the covering agent is about 50% (w/w) or lower, preferably about 5 to about 40% (w/w), more preferably about 4 to about 35% (w/w). Herein, the % (w/w) indicates % by weight relative to a covering agent composition in which a solvent (e.g. water, lower alcohol such as methanol, ethanol, etc.) is removed from a covering agent solution.

The sustained-release preparation is produced by preparing a core containing a drug, and covering the resulting core with a covering agent in which a water-insoluble substance or a swelling polymer is heat-melted or dissolved or dispersed in a solvent, as exemplified below.

### I. Preparation of Core Containing Drug

The form of a core containing a drug to be covered with a covering agent (hereinafter, sometimes, simply referred to as the core) is not particularly limited, but the core is preferably formed into a particulate shape such as a granule and a fine particle.

When the core is a granule or a fine particle, the average particle diameter is preferably about 150 to 2,000 μm, more preferably about 500 to about 1,400 μm.

The core can be prepared according to a conventional manner. For example, the core is prepared by mixing a drug with an appropriate excipient, binder, disintegrating agent, lubricant, stabilizer, etc., and formulating the resulting mixture into a core by wet extrusion granulation or fluidized layer granulation.

The content of the drug in the core is about 0.5 to about 95% (w/w), preferably about 5.0 to about 80% (w/w), more preferably about 30 to about 70% (w/w).

Examples of the excipient to be contained in the core include saccharides such as white sugar, lactose, mannitol, glucose, etc., starch, crystalline cellulose, calcium phosphate, corn starch, and the like. Inter alia, crystalline cellulose, and corn starch are preferable.

Examples of the binder include polyvinyl alcohol, hydroxylpropylcellulos, polyethylene glycol, polyvinylpyrrolidone, Pluronic F68, gum arabic, gelatin, starch, etc. Examples of the disintegrating agent include calcium carboxymethylcellulose (ECG505), sodium croscarmellose (Ac-Di-Sol), crosslinked polyvinylpyrrolidone (crospovidone), and low substituted hydroxypropylcellulose (L-HPC), etc. Inter alia, hydroxypropylcellulose, polyvinyl pyrrolidone, and low substituted hydroxypropylcellulose are preferable. Examples of the lubricant or an aggregation preventing agent include talc, magnesium stearate and its inorganic salt, etc. As the lubricant, polyethylene glycol, etc. is used. Examples of the stabilizer include acids such as tartaric acid, citric acid, succinic acid, fumaric acid, maleic acid, etc.

In addition to the above method, the core can be also prepared, for example, by rolling granulation, pan coating, fluidized layer coating or melt granulation, wherein a drug or a mixture thereof with an excipient or a lubricant is added by portions, while a binder dissolved in a suitable solvent such as a lower alcohol (e.g. methanol, ethanol, etc.) is sprayed on an inert carrier particle which is to be a center of the core. Examples of the inert carrier particle include a particle prepared with white sugar, lactose, starch, crystalline cellulose, or wax, and its average particle diameter is preferably about 100 μm to about 1,500 μm.

In order to separate the drug contained in the core and the covering agent, a surface of the core may be covered with a protecting agent. Examples of the protecting agent include the aforementioned hydrophilic substance, a water-insoluble substance, etc. As the protecting agent, preferably, polyethylene glycol, and polysaccharides having a hydroxyalkyl group or a carboxyalkyl group, more preferably hydroxypropylmethylcellulose, and hydroxypropylcellulose are used. The protecting agent may contain an acid such as tartaric acid, citric acid, succinic acid, fumaric acid maleic acid, etc. as a stabilizer, as well as a lubricant such as talc, etc. When the protecting agent is used, its covering amount is about 1 to about 15% (w/w), preferably about 1 to about 10% (w/w), more preferably about 2 to about 8% (w/w) relative to the core.

The core can be covered with the protecting agent by a conventional coating method and, specifically, the core can be covered with the protecting agent, for example, by spray-coating, fluidized layer coating, pan coating, etc.

II. Covering Core with Covering Agent

The sustained-release preparation is prepared by covering the core obtained in the above I with a covering agent solution in which the water-insoluble substance and the pH dependent swelling polymer, and a hydrophilic substance are heat-melted or dissolved or dispersed in a solvent.

Examples of a covering method with the covering agent solution include spray coating, etc.

The compositional ratio of the water-insoluble substance, the swelling polymer or the hydrophilic substance in the covering agent solution is appropriately selected so that the content of each component in a coated film becomes the aforementioned content.

The covering amount of the covering agent is about 1 to about 90% (w/w), preferably about 5 to about 50% (w/w), more preferably about 5 to about 35% (w/w) relative to the core (without the covering amount of the protecting agent).

Examples of the solvent for the covering agent solution include water and an organic solvent, and they can be used alone or as a mixture of them. The mixing ratio of water and an organic solvent (water/organic solvent:weight ratio) in case using a mixture can vary in a range of 1 to 100%, preferably 1 to about 30%. The organic solvent is not particularly limited as far as it can dissolve the water-insoluble substance. Examples thereof include lower alcohols such as methyl alcohol, ethyl alcohol, isopropyl alcohol, n-butyl alcohol, etc., lower alkanones such as acetone, etc., acetonitrile, chloroform, methylene chloride, etc. Among them, lower alcohols are preferable, and ethyl alcohol and isopropyl alcohol are particularly preferable. Water and a mixture of water and an organic solvent are preferably used as a solvent for the covering agent. At this time, if necessary, an acid such as tartaric acid, citric acid, succinic acid, fumaric acid, maleic acid, etc. may be added to the covering agent solution in order to stabilize the covering agent solution.

Spray coating can be carried out by a conventional manner, specifically, can be carried out by spray-coating the core, for example, by fluidized layer coating, pan coating, etc. At this time, if necessary, a lubricant such as talc, titanium oxide, magnesium stearate, calcium stearate, light silicic acid anhydride, etc. may be added, and a plasticizer such as glycerin fatty acid ester, hydrogenated castor oil, triethyl citrate, cetyl alcohol, stearyl alcohol, etc. may be added.

After covered with the covering agent, if necessary, an antistatic agent such as talc, etc. may be mixed therein.

The rapid-release preparation may be liquid (solution, suspension, emulsion, etc.) or solid (particulate pill, tablet, etc.). An oral preparation, and a parenteral preparation such as an injectable preparation are used, and an oral preparation is preferable.

The rapid-release preparation may usually contain a carrier, an additive and excipient which are conventionally used in the pharmaceutical field (hereinafter, sometimes, abbreviated as the excipient) in addition to the drug, i.e., the active component. A pharmaceutical excipient to be used is not particularly limited as far as it is conventionally used as a pharmaceutical excipient. Examples of the excipient for an oral solid preparation include lactose, starch, corn starch, crystalline cellulose (Avicel PH101, manufactured by Asahi Chemical Industry Co., Ltd.), powdered sugar, granulated sugar, mannitol, light silicic acid anhydride, magnesium carbonate, calcium carbonate, L-cysteine, etc., preferably, corn starch, mannitol, etc. These excipients can be used alone, or by combining two or more thereof. The content of the excipient is, for example, about 4.5 to about 99.4 w/w %, preferably about 20 to about 98.5 w/w %, more preferably about 30 to about 97 w/w % relative to the total amount of the rapid-release preparation.

The content of the drug in the rapid-release preparation can be appropriately selected from a range of about 0.5 to about 95%, preferably about 1 to about 60% relative to the total amount of the rapid-release preparation.

When the rapid-release preparation is an oral solid preparation, it usually contains a disintegrating agent in addition to the aforementioned components. Examples of such the disintegrating agent include calcium carboxymethylcellulose (ECG-505, manufactured by Gotoku Yakuhin), sodium croscarmellose (e.g. Acdisol, manufactured by Asahi Chemical Industry Co., Ltd.), crospovidone (e.g. Corridone CL, manufactured by BASF), low substituted hydroxypropylcellulose (manufactured by Shin-Estu Chemical Co., Ltd.), carboxymethylstarch (Matsutani Chemical Industry Co., LTd.), sodium carboxymethylstarch (Exprotab, manufactured by Kimura Industry), partially gelatinized starch (PCS, manufactured by Asahi Chemical Industry Co., Ltd), etc. For example, a disintegrating agent which disintegrates a granule by contacting with water to absorb water, to be swollen, or make a channel between the active component and the excipient constituting the core, can be used. These disintegrating agents can be used alone, or by combining two or more thereof. The blending amount of the disintegrating agent is appropriately selected depending on a kind and a blending amount of the drug used, and pharmaceutical designe of release ability, and is for example about 0.05 to about 30 w/w %, preferably about 0.5 to about 15 w/w % relative to the total amount of the rapid-release preparation.

When the rapid-release preparation is an oral solid preparation, optionally, the solid preparation may further contain a conventional additive in addition to the aforementioned composition. Examples of such the additive include a binder (e.g. sucrose, gelatin, gum arabic powder, methyl cellulose, hydroxypropylcellulose, hydroxypropylmethylcellulose, carboxymethylcellulose, polyvinylpyrrolidone, pullulan, dextrin, etc.), a lubricant (e.g. polyethylene glycol, magnesium stearate, talc, light silicic acid anhydride (e.g. Aerosil (Nippon Aerosil), etc.), a surfactant (e.g. anionic surfactant such as sodium alkyl sulfate, etc., nonionic surfactant such as polyoxyethylene fatty acid ester, polyoxyethylene sorbitan fatty acid ester, polyoxyethylene castor oil derivative, etc.), a coloring agent (e.g. tar pigments, caramel, bengala, titanium oxide, riboflavins, etc.), if necessary, a corrigent (e.g. sweetener, perfume, etc.), an adsorbing agent, an antiseptic, a wetting agent, an antistatic agent, etc. In addition, as a stabilizer, an organic acid such as tartaric acid, citric acid, succinic acid, fumaric acid, etc. may be added.

As the binder, hydroxypropylcellulose, polyethylene glycol, polyvinylpyrrolidone, etc. are preferably used.

The rapid-release preparation can be prepared by mixing the aforementioned respective components, if necessary, further kneading the mixture, and molding the mixture according to conventional pharmaceutical preparation manufacturing technique. The above mixing is carried out by a generally used method such as mixing, kneading, etc. Specifically, for example, when the rapid-release preparation is molded into particles, the preparation can be prepared by mixing components using a vertical granulator, a universal kneading machine (manufactured by Hata Tekkosyo), or a fluidized layer granulating machine FD-5S (manufactured by Powlex) by the same procedure as the process for preparing the core of the sustained-release preparation and, thereafter, granulating the resulting mixture by wet extrusion granulation, fluidized layer granulation, etc.

The thus obtained rapid-release preparation and sustained-release preparation, as they are, or after separately formulated into preparations together with pharmaceutical excipients, may be formulated into different preparations which are administered simultaneously, or administered by combining them at an arbitrary administration interval(s). Alternatively, both, as they are, or together with a pharmaceutical excipient, may be formulated into a single oral preparation (e.g. granule, fine granule, tablet, capsule, etc.). Further, both preparations are prepared in the form of granules or fine granules, and may be filled into the same capsule to obtain an oral preparation.

[3] Sublingual, Buccal or Oral Cavity Rapid-Disintegrating Agent and its Production

A sublingual tablet, a buccal preparation, and an oral cavity rapid-disintegrating agent may be a solid preparation such as a tablet, or may be an oral cavity mucosa applying tablet (film).

As a sublingual tablet, a buccal or an oral cavity rapid-disintegrating agent, a preparation containing the present androgen receptor antagonist or the combined use drug and an excipient is preferable. Further, it may contain aids such as a lubricant, an isotonic, a hydrophilic carrier, a water-dispersible polymer, a stabilizer, etc. Furthermore, in order to facilitate absorption, and enhance bioavailability, it may contain β-cyclodextrin or a β-cyclodextrin derivative (e.g. hydroxypropyl-β-cyclodextrin etc.), etc.

Examples of the excipient include lactose, white sugar, D-mannitol, starch, crystalline cellulose, light silicic acid anhydride, etc. Examples of the lubricant include magnesium stearate, calcium stearate, talc, colloidal silica, etc. Particularly, magnesium stearate and colloidal silica are preferable. Examples of the isotonic include sodium chloride, glucose, fructose, mannitol, sorbitol, lactose, saccharose, glycerin, urea, etc. Particularly, mannitol is preferable. Examples of the hydrophilic carrier include swelling hydrophilic carriers such as crystalline cellulose, ethylcellulose, crosslinking polyvinylpyrrolidone, light silicic acid anhydride, silicic acid, dicalcium phosphate, calcium carbonate, etc. Particularly, crystalline cellulose (e.g. microcrystalline cellulose etc.) is preferable. Examples of the water-dispersible polymer include gum (e.g. tragacanth gum, gum acacia, guar gum), alginate (e.g. sodium alginate), cellulose derivative (e.g. methylcellulose, carboxymethylcellulose, hydroxymethylcellulose, hydroxypropylcellulose, hydroxypropylmethylcellulose) gelatin, water-soluble starch, polyacrylic acid (e.g. carbomer), polymethacrylic acid, polyvinyl alcohol, polyethylene glycol, polyvinylpyrrolidone, polycarbophil, ascorbate, palmitate, etc. Hydroxypropylmethylcellulose, polyacrylic acid, alginate, gelatin, carboxymethylcellulose, polyvinylpyrrolidone, polyethylene glycol, etc. are preferable. Particularly, hydroxypropylmethylcellulose is preferable. Examples of the stabilizer include cysteine, thiosorbitol, tartaric acid, citric acid, sodium carobonate, ascorbic acid, glycine, sodium sulfite, etc. Particularly, citric acid and ascorbic acid are preferable.

The sublingual tablet, the buccal or the oral cavity rapid-disintegrating agent can be prepared by mixing the present androgen receptor antagonist or the combined use drug and an excipient by a known per se method. Further, if desired, the aforementioned aids such as a lubricant, an isotonic, a hydrophilic carrier, a water-dispersible polymer, a stabilizer, a coloring agent, a sweetener, an antiseptic, etc. may be mixed therein. The aforementioned components are mixed simultaneously or separately at a certain internal, followed by compressing and molding under pressure to obtain the sublingual tablet, the buccal tablet or the oral cavity rapid-disintegrating tablet. In order to obtain suitable hardness, if necessary, the tablet may be prepared by wetting and swelling with a solvent such as water and alcohol before and/or after the compressing and molding step, and drying the molded material.

For molding into the mucosa applying tablet (film), the present androgen receptor antagonist or the combined use drug, the aforementioned water-dispersible polymer (preferably, hydroxypropylcellulose, hydroxypropylmethylcellulose), and an excipient, etc. are dissolved in a solvent such as water, and the resulting solution is cast to obtain a film. Further, an additive such as a plasticizer, a stabilizer, an antioxidant, a preservative, a coloring agent, a buffer, a sweetener, etc. may be added. For imparting suitable elasticity to the film, glycols such as polyethylene glycol and propylene glycol may be added, or for enhancing adherence of the film to a mucosal lining in the oral cavity, a bioadhesive polymer (e.g. polycarbophil, carbopol, etc.) may be added. Casting is carried out by pouring a solution on a non-adhesive surface, spreading it to a uniform thickness (preferably about 10 to 1000 micron) with a coating equipment such as a doctor blade, etc., and drying the solution to form a film. The thus formed film may be dried at room temperature or with worming, and cut into a desired surface area.

Examples of the preferable oral cavity rapid-disintegrating agent include a solid rapid-diffusing agent comprising a network of the present androgen receptor antagonist or the combined use drug, and a water-soluble or water-diffusible carrier which is inert to the present androgen receptor antagonist or the combined use drug. The network is obtained by sublimating a solvent from a solid composition comprising a solution in which the present androgen receptor antagonist or the combined use drug is dissolved in a suitable solvent.

It is preferable that the composition of the oral cavity rapid-disintegrating agent contains a matrix forming agent and a secondary component in addition to the present androgen receptor antagonist or the combined use drug.

The matrix forming agent include substances derived from dextrins; animal proteins or vegetable proteins such as gelatins, soybean, wheat, psyllium seed proteins, etc.; gummy substances such as gum arabic, guar gum, agar, xanthan, etc.; polysaccharides; alginic acids; carboxymethylcelluloses; carrageenans; dextrans; pectins; synthetic polymers such as polyvinyl pyrrolidone; gelatin-gum arabic complex; etc. Further examples include saccharides such as mannitol, dextrose, lactose, galactose, trehalose, etc.; cyclic saccharides such as cyclodextrin, etc.; inorganic salts such as sodium phosphate, sodium chloride, aluminum silicate, etc.; amino acids having 2 to 12 carbon atoms such as glycine, L-alanine, L-aspartic acid, L-glutamic acid, L-hydroxyproline, L-isoleucine, L-leucine, L-phenylalanine, etc.

One or more kinds of matrix forming agents can be added to a solution or a suspension before solidification. Such the matrix forming agent may be present in addition to a surfactant or may be present without a surfactant. The matrix forming agent helps to maintain the diffusion state of the present androgen receptor antagonist or the combined use drug in its solution or suspension in addition to formation of the matrix.

The composition may contain the secondary component such as a preservative, an antioxidant, a surfactant, a viscosity increasing agent, a coloring agent, a pH adjusting agent, a flavor, a sweetener a taste masking agent, etc. Examples of the suitable coloring agent include red, black and yellow iron oxides, and FD&C dyes such as FD&C Blue No. 2 and FD&C Red No. 40 of Ellis and Everald, etc. Examples of the suitable flavor include mint, raspberry licorice, orange, lemon, grapefruit, caramel, vanilla, cherry grape flavor, etc. and a combination thereof. Examples of the suitable pH adjusting agent include citric acid, tartaric acid, phosphoric acid, hydrochloric acid, maleic acid, etc. Examples of the suitable sweetener include aspartame, acesulfame K, taumatin, etc. Examples of the suitable taste masking agent include sodium bicarbonate, ion exchange resin, cyclodextrin inclusion compound, adsorbing substance, microcapsulated apomorphine, etc.

Usually, the preparation contains about 0.1 to about 50% by weight, preferably about 0.1 to about 30% by weight of the present androgen receptor antagonist or the combined use drug. Preferably, the preparation is the above sublingual, buccal, etc., which can dissolve 90% or more of the present androgen receptor antagonist or the combined use drug (in water) within about 1 minute to about 60 minutes, preferably about 1 minute to about 15 minutes, more preferably about 2 minutes to about 5 minutes, or the oral cavity rapid-disintegrating agent which is disintegrated within 1 to 60 seconds, preferably 1 to 30 seconds, more preferably 1 to 10 seconds after placed into the oral cavity.

The content of the excipient relative to the total preparation is about 10 to about 99% by weight, preferably about 30 to about 90% by weight. The content of β-cyclodextrin or a β-cyclodextrin derivative relative to the total preparation is 0 to about 30% by weight. The content of the lubricant relative to the total preparation is about 0.01 to about 10% by weight, preferably about 1 to about 5% by weight. The content of the isotonic relative to the total preparation is about 0.1 to about 90% by weight, preferably about 10 to about 70% by weight. The content of the hydrophilic carrier relative to the total preparation is about 0.1 to about 50% by weight, preferably about 10 to about 30% by weight. The content of the water-dispersible polymer relative to the total preparation is about 0.1 to about 30% by weight, preferably about 10 to about 25% by weight. The content of the stabilizer relative to the total preparation is about 0.1 to about 10% by weight, preferably about 1 to about 5% by weight. If necessary, the preparation may further contain an additive such as a coloring agent, a sweetener, an antiseptic, etc.

A dose of the present combination use preparation varies depending on a kind of the compound (I), an age, a weight, symptom, a dosage form, an administration method, and an administration term, but for example, the preparation is intravenously administered at about 0.01 to about 1000 mg/kg, preferably about 0.01 to about 100 mg/kg, more preferably about 0.1 to about 100 mg/kg, inter alia, about 0.1 to about 50 mg/kg, among them, about 1.5 to about 30 mg/kg in terms of the present androgen receptor antagonist or the combined use drug per a sepsis patient (adult, weight about 60 kg). This is administered once or by dividing into a few times a day. Of course, since the dose varies under various conditions as described above, a dose smaller than the aforementioned dose may be sufficient in some cases, and a dose exceeding the aforementioned range may be required in some cases.

The amount of the combined use drug may be set in such a range that side effect is not problematic. A dose per day in terms of the combined use drug varies depending on a degree of symptom, an age, a sex, a weight, and a difference in sensitivity of an administration subject, an administration term and intervals, nature, compounding and a kind of a pharmaceutical preparation and a kind of an active ingredient, and is not limited. The amount of the drug is usually about 0.001 to about 2000 mg, preferably about 0.01 to about 500 mg, more preferably about 0.1 to about 100 mg per 1 kg of a mammal in the case of oral administration. Usually, this is administered once or by dividing into up to four times a day

When the present combination use preparation is administered, the combined use drug may be administered first and, thereafter, the present androgen receptor antagonist may be administered, or the present androgen receptor antagonist may be administered first and, thereafter, the combined use drug may be used, although they may be administered at the same time. When both are administered at different times, the interval varies depending on a particular active component to be administered, a dosage form, and an administration method, but, for example, when the combined use drug is administered first, the present androgen receptor antagonist is administered within 1 minute to 3 days, preferably 10 minutes to 1 day, more preferably 15 minutes to 1 hour after administration of the combined use drug. When the present androgen receptor antagonist is administered first, the combined use drug is administered within 1 minute to 1 day, preferably 10 minutes to 6 hours, more preferably 15 minutes to 1 hour after administration of the present androgen receptor antagonist.

As a preferable administration method, for example, about 0.001 to 200 mg/kg of the combined use drug in the form of an oral preparation is orally administered and, after 15 minutes, about 0.005 to 100 mg/kg of the present androgen receptor antagonist in the form of an oral preparation is orally administered as one day dosage.

Hereinafter, the present invention will be illustrated in more detail by way of Reference Examples, Examples, Preparation Examples and Test Examples, But the Present invention is not limited thereto.

“Room temperature” in the following Reference Examples indicates usually about 10° C. to about 35° C. The “percents (%)” are by weight unless otherwise stated. The yield shows mol/mol %. NMR spectrum indicates proton NMR, and this was measured with a 200 MHz or 300 MHz-type spectrometer using tetramethylsilane as an internal standard, and a 5 value is expressed in ppm. Unless otherwise stated, ¹H-NMR indicates a value measured with a 300 MHz-type spectrometer.

Other abbreviations used herein indicate the following meanings.
s: singlet
brs: broad singlet
d: doublet
t: triplet
q: quartet
dd: double doublet
ddd: double double doublet
dt: double triplet
m: multiplet
br: broad
CDCl₃: heavy chloroform
DMSO-d₆: heavy dimethylsulfoxide
¹H-NMR: proton nuclear magnetic resonance
Me: methyl
Et: ethyl
i-Pr: isopropyl
boc: tert-butyloxycarbonyl
THF: tetrahydrofuran
DMSO: dimethyl sulfoxide
DMF: N,N-dimethylformamide
DMAP: 4-(dimethylamino)pyridine
WSC: 1-ethyl-3-(3-dimethylaminopropyl)carbodiimide hydrochloride

### REFERENCE EXAMPLE 1

### 4-(4-Cyanophenyl)-5-methyl-2-propyl-1H-pyrrole-3-carboxylic acid

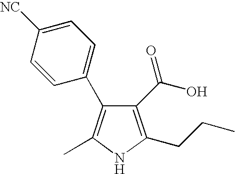

A mixture of benzyl 3-oxohexanoate (6.40 g) synthesized by a known method, potassium hydroxide (776 mg) and THF (50 ml) was stirred at room temperature for 20 minutes, and cooled to 0° C. 4-(2-Nitroprop-1-en-1-yl)benzonitrile (2.60 g) synthesized by a known method was added, this was stirred at 0° C. for 1 hour, and poured into an aqueous sodium chloride solution, followed by extraction with ethyl acetate. The extract was washed with an aqueous sodium chloride solution, dried (sodium sulfate) and concentrated to obtain an oil. A mixture of this oil, methanol (10 ml), water (1 ml) and hydrochloric acid (1 ml) was stirred at 75° C. for 2 hours. The reaction mixture was cooled to room temperature, and concentrated, and the residue was distributed between ethyl acetate and water. The organic layer was washed with a sodium bicarbonate solution and an aqueous sodium chloride solution, dried (sodium sulfate) and concentrated. The residue was subjected to silica gel column chromatography (ethyl acetate-hexane) to obtain benzyl 4-(4-cyanophenyl)-5-methyl-2-propyl-1H-pyrrole-3-carboxylate. This compound was used in the following reaction without purification.

A mixture of benzyl 4-(4-cyanophenyl)-5-methyl-2-propyl-1H-pyrrole-3-carboxylate, 10% palladium carbon (50% hydrous product 294 mg), THF (105 ml) and methanol (35 ml) was stirred at room temperature for 3 hours under a hydrogen atmosphere. A catalyst was filtered off using Celite, and washed with methanol. The washings and the mother liquor were combined, concentrated, and washed with ethyl acetate to obtain the desired product (2.42 g) as crystals.

¹H-NMR (DMSO-d₆) δ: 0.90 (3H, t), 1.54-1.66 (2H, m), 2.05 (3H, s), 2.80 (2H, t), 7.35 (2H, d), 7.70 (2H, d), 11.14 (1H, s), 11.35 (1H, br.s).

According to the same manner as that shown in Reference Example 1, the following compounds were obtained.

### 4-(4-Cyanophenyl)-2-cyclopropyl-5-methyl-1H-pyrrole-3-carboxylic acid

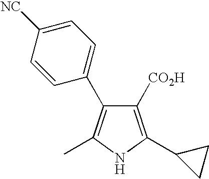

¹H-NMR (DMSO-d₆) δ: 0.78-0.83 (2H, m), 0.89-0.95 (2H, m), 2.02 (3H, s), 2.65-2.74 (1H, m), 7.35 (2H, d), 7.73 (2H, d), 10.63 (1H, s).

### 4-(4-Cyanophenyl)-5-ethyl-2-methyl-1H-pyrrole-3-carboxylic acid

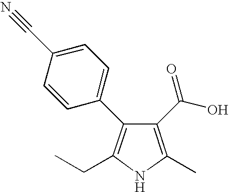

¹H-NMR (DNSO-d₆) δ: 1.07 (3H, t), 2.37 (2H, q), 2.41 (3H, s), 7.34 (2H, d), 7.72 (2H, d), 10.98 (1H, s), 11.19 (1H, s).

### 4-(4-Cyanophenyl)-2,5-dimethyl-1H-pyrrole-3-carboxylic acid

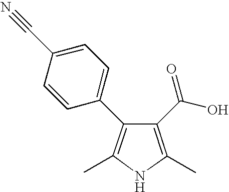

¹H-NMR (DMSO-d₆) δ 2.05 (3H, s), 2.39 (3H, s), 7.35 (2H, d), 7.71 (2H, d), 11.22 (1H, s), 11.43 (1H, s).

### 4-(4-Cyanophenyl)-2-ethyl-5-methyl-1H-pyrrole-3-carboxylic acid

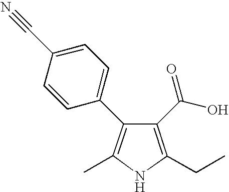

¹H-NMR (DMSO-d₆) δ: 1.17 (3H, t), 2.06 (3H, s), 2.84 (2H, q), 7.37 (2H, d), 7.73 (2H, d), 11.19 (1H, s).

### REFERENCE EXAMPLE 2

### 4-(2-Methyl-5-propyl-1H-pyrrol-3-yl)benzonitrile

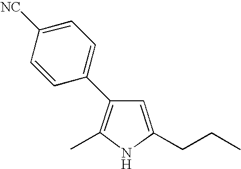

A mixture of 4-(4-cyanophenyl)-5-methyl-2-propyl-1H-pyrrole-3-carboxylic acid (2.42 g) and trifluoroacetic acid (5 ml) was stirred at room temperature for 40 minutes. The reaction mixture was concentrated to obtain the residue, which was diluted with ethyl acetate, washed with a sodium bicarbonate solution and an aqueous sodium chloride solution, dried (sodium sulfate) and concentrated. The residue was purified by silica gel column chromatography (ethyl acetate-hexane) to obtain the desired product (1.97 g) as crystals.

¹H-NMR (CDCl₃) δ: 0.99 (3H, t), 1.66 (2H, sextet), 2.40 (3H, s), 2.55 (2H, t), 6.05 (1H, d), 7.44-7.48 (2H, m), 7.57-7.61 (2H, m), 7.78 (1H, br.s).

According to the same manner as that shown in Reference Example 2, the following compounds were obtained.

### 4-(5-Cyclopropyl-2-methyl-1H-pyrrol-3-yl)benzonitrile

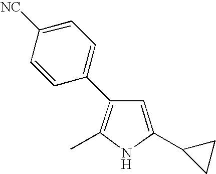

¹H-NMR (CDCl₃) δ: 0.61-0.67 (2H, m), 0.82-0.88 (2H, m), 1.75-1.82 (1H, m), 2.39 (3H, s), 5.99 (1H, d), 7.45 (2H, d), 7.60 (2H, d), 7.89 (1H, s).

### 4-(2-Ethyl-5-methyl-1H-pyrrol-3-yl)benzonitrile

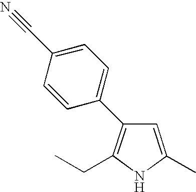

¹H-NMR (DNSO-d₆) δ: 1.18 (3H, t), 2.59 (3H, s), 2.69 (2H, q), 5.94 (1H, d), 7.45 (2H, d), 7.71 (2H, d), 10.64 (1H, s).

### 4-(2,5-Dimethyl-1H-pyrrol-3-yl)benzonitrile

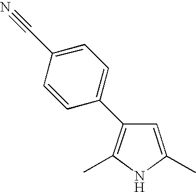

¹H-NMR (CDCl₃) δ 2.27 (3H, s), 2.40 (3H, s), 6.04 (1H, d), 7.46 (2H, d), 7.61 (2H, d), 7.76 (1H, br s).

### 4-(5-Ethyl-2-methyl-1H-pyrrol-3-yl)benzonitrile

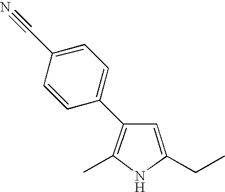

¹H-NMR (CDCl₃) δ: 1.27 (3H, t), 2.41 (3H, s), 2.62 (2H, q), 6.06 (1H, d), 7.46-7.49 (2H, m), 7.59-7.62 (2H, m), 7.80 (1H, br.s).

### REFERENCE EXAMPLE 3

### 4-(4-Cyanophenyl)-5-methyl-2-propyl-1H-pyrrole-3-carbonitrile

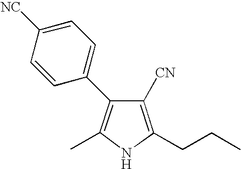

A mixture of chlorosulfonyl isocyanate (0.82 ml) and acetonitrile (4 ml) was added dropwise to a mixture of 4-(2-methyl-5-propyl-1H-pyrrole-3-yl)benzonitrile (1.93 g), DMF (1.8 ml) and acetonitrile (12 ml) at 0° C. After stirred at 0° C. for 30 minutes, the reaction was poured into a sodium bicarbonate solution, and this was extracted with ethyl acetate. The extract was washed with an aqueous sodium chloride solution, and purified by silica gel column chromatography (ethyl acetate) to obtain the desired product (2.14 g) as crystals.

¹H-NMR (CDCl₃) δ: 1.01 (3H, t), 1.67-1.79 (2H, m), 2.32 (3H, s), 2.75 (2H, t), 7.51-7.55 (2H, m), 7.68-7.72 (5H, m), 8.27 (1H, br.s).

According to the same manner as that shown in Reference Example 3, the following compounds were obtained.

### 4-(4-Cyanophenyl)-2-cyclopropyl-5-methyl-1H-pyrrole-3-carbonitrile

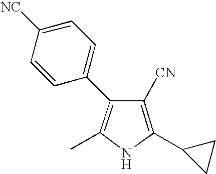

¹H-NMR (CDCl₃) δ: 0.89-0.94 (2H, m), 1.03-1.09 (2H, m), 1.98-2.03 (1H, m), 2.29 (3H, s), 7.50 (2H, d), 7.68 (2H, d), 8.23 (1H, s).

### 4-(4-Cyanophenyl)-5-ethyl-2-methyl-1H-pyrrole-3-carbonitrile

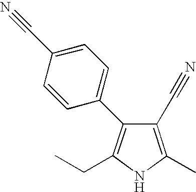

¹H-NMR (CDCl₃) δ: 1.23 (3H, t), 2.45 (3H, s), 2.67 (2H, q), 7.49 (2H, d), 7.68 (2H, d), 8.46 (1H, s).

### 4-(4-Cyanophenyl)-2-ethyl-5-methyl-1H-pyrrole-3-carbonitrile

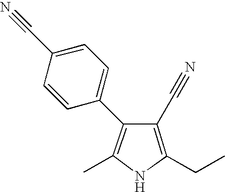

¹H-NMR (DMSO-d₃) δ: 1.24 (3H, t), 2.26 (3H, s), 2.69 (2H, q), 7.57 (2H, d), 7.89 (2H, d), 11.72 (1H, s).

### 3-(4-Cyanophenl)-5-methyl-1H-pyrrole-2-carbonitrile

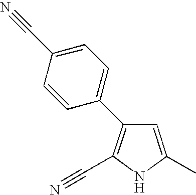

¹H-NMR (CDCl₃) δ 2.36 (3H, s), 6.27 (1H, dd), 7.59 (2H, d), 7.76 (2H, d), 8.74 (1H, s).

### Tert-butyl 2-cyano-4-(4-cyanophenyl)-5-methyl-1H-pyrrole-3-carboxylate

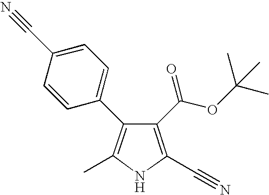

¹H-NMR (CDCl₃) δ1.44 (9H, s), 2.19 (3H, s), 7.37 (2H, d), 7.69 (2H, d), 9.05 (1H, s).

### REFERENCE EXAMPLE 4

### 3-Chloro-6-methylpyridine-2-carbonitrile

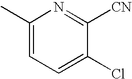

A mixture of 3-chloro-6-methylpyridine-2-carbonitrile 1-oxide (3.0 g), trimethylsilyl cyanide (3.1 ml), N,N-dimethylcarbamoyl chloride (1.9 ml), and dichloromethane (40 ml) was heated at 50° C. for 14 hours. After an aqueous saturated sodium chloride solution (100 ml) was added to the reaction mixture, this was extracted with ethyl acetate, and the ethyl acetate layer was washed with an aqueous saturated sodium chloride solution, dried over magnesium sulfate, and concentrated. The residue was purified by silica gel column chromatography (ethyl acetate-hexane) to obtain the desired product (1.67 g) as a solid.

¹H-NMR (CDCl₃) δ 2.59 (3H, s), 7.33 (1H, d), 7.73 (1H, d).

### REFERENCE EXAMPLE 5

### 3-Chloro-6-methylpyridine-2-carboxylic acid

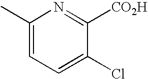

A mixture of 3-chloro-6-methylpyridine-2-carbonitrile (1.52 g), a 1N aqueous sodium hydroxide solution (2 ml) and ethanol (2 ml) was heated at 80° C. for 2 hours. After 1N hydrochloric acid (4 ml) was added to the reaction mixture, this was extracted with ethyl acetate, and the ethyl acetate layer was washed with an aqueous saturated sodium chloride solution, dried over magnesium sulfate, and concentrated. The residue was crystallized from ethyl acetate to obtain the desired product (1.10 g) as crystals.

¹H-NMR (CDCl₃) δ 2.56 (1H, s), 2.61 (3H, s), 7.38 (1H, d), 7.81 (1H, d).

### REFERENCE EXAMPLE 6

### Methyl 3-Chloro-6-methylpyridine-2-carboxylate

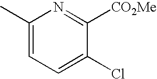

A mixture of 3-chloro-6-methylpyridine-2-carboxylic acid (1.10 g), and a 1N hydrogen chloride solution in methanol (6 ml) was heated at 80° C. for 20 hours. After an aqueous saturated sodium bicarbonate solution (20 ml) was added to the reaction mixture, this was extracted with ethyl acetate, and the ethyl acetate layer was washed with an aqueous saturated sodium chloride solution, dried over magnesium sulfate, and concentrated. The residue was purified by silica gel column chromatography (ethyl acetate-hexane) to obtain the desired product (1.67 g) as an oil.

¹H-NMR (CDCl₃) δ 2.59 (3H, s), 4.00 (3H, s), 7.23 (1H, d), 7.68 (1H, d).

According to the same manner as that shown in Reference Example 6, the following compounds were obtained.

### Methyl 2,5-dichloroisonicotinate

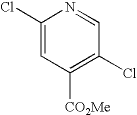

¹H-NMR (CDCl₃) δ 4.00 (3H, s), 7.42 (1H, d), 7.77 (1H, d). Methyl 2-chloro-5-methylbenzoate

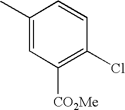

¹H-NMR (200 mHz, CDCl₃) δ 2.35 (3H, s), 3.92 (3H, s), 7.21 (1H, dd), 7.33 (1H, d), 7.63 (1H, d).

### REFERENCE EXAMPLE 7

### Methyl 5-Chloro-2-methylisonicotinate

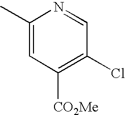

A 3N methylmagnesium bromide solution in ether (5.6 ml) was added dropwise to a mixture of methyl 2,5-dichloroisonicotinate (2.90 g), iron (III) acetylacetonate (0.25 g), THF (85 ml) and N-methylpyrrolidone (8.5 ml) under ice-cooling, and this was stirred at the same temperature for 1 hour. After an aqueous saturated sodium chloride solution (100 ml) was added to the reaction mixture, this was extracted with ethyl acetate, and the ethyl acetate layer was washed with an aqueous saturated sodium chloride solution, dried over magnesium sulfate, and concentrated. The residue was purified by silica gel column chromatography (ethyl acetate-hexane) to obtain the desired product (1.82 g) as an oil.

¹H-NMR (CDCl₃) δ 2.52 (3H, s), 3.99 (3H, s), 7.74 (1H, s), 8.34 (1H, s).

### REFERENCE EXAMPLE 8

### Ethyl 2-chloro-5-methylnicotinate

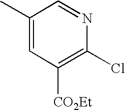

Phosphorus oxychloride (36 ml) was added dropwise to a mixture of ethyl (2E)-2-cyanopent-2-ene (30 g) and DMF (76 ml) under ice-cooling. This mixture was stirred at 80° C. for 2 hours. After ice water (200 ml) was added to the reaction mixture, this was extracted with ethyl acetate, and the ethyl acetate layer was washed with an aqueous saturated sodium chloride solution, dried over magnesium sulfate, and concentrated. The residue was purified by silica gel column chromatography (ethyl acetate-hexane) to obtain the desired product (21.1 g) as an oil.

¹H-NMR (CDCl₃) δ 1.42 (3H, t), 2.37 (3H, s), 4.42 (2H, q), 7.96 (1H, d), 8.32 (1H, d).

According to the same manner as that shown in Reference Example 8, the following compound was obtained.

### Isopropyl 2-chloro-5-methylnicotinate

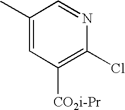

¹H-NMR (CDCl₃) δ 1.40 (6H, d), 2.37 (3H, s), 5.28 (1H, sept), 7.91 (1H, d), 8.31 (1H, d).

### REFERENCE EXAMPLE 9

### Ethyl 5-(bromomethyl)-2-chloronicotinate

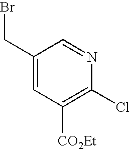

A mixture of ethyl 2-chloro-5-methylnicotinate (14.7 g), 2,2′-azobis(isobutyronitrile) (1.31 g), N-bromosuccinimide (17.1 g) and carbon tetrachloride (200 ml) was heated at 90° C. for 5 hours. After an aqueous saturated sodium chloride solution (100 ml) was added to the reaction mixture, this was extracted with ethyl acetate, and the ethyl acetate layer was washed with an aqueous saturated sodium chloride solution, dried over magnesium sulfate, and concentrated. The residue was purified by silica gel column chromatography (ethyl acetate-hexane) to obtain the desired product (10.5 g) as an oil.

¹H-NMR (CDCl₃) δ 1.4-1.5 (3H, m), 4.3-4.5 (4H, m), 7.97 (1H, d), 8.19 (1H, d)

According to the same manner as that shown in Reference Example 9, the following compounds were obtained.

### Methyl 5-(bromomethyl)-2-chlorobenzoate

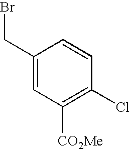

¹H-NMR (200 mHz, CDCl₃) δ 3.94 (3H, s), 4.46 (2H, s), 7.44 (2H, d), 7.86 (1H, s).

### Methyl 2-(bromomethyl)-5-chloronicotinate

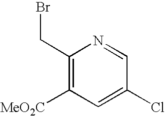

¹H-NMR (CDCl₃) δ 3.99 (3H, s), 5.00 (2H, s), 8.27 (1H, d), 8.66 (1H, d).

### Methyl 2-(bromomethyl)-5-chloroisonicotinate

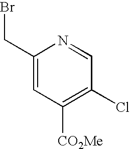

¹H-NMR (CDCl₃) δ 4.00 (3H, s), 4.86 (2H, s), 7.81 (1H, s), 8.52 (1H, s).

### Methyl 6-(bromomethyl)-3-chloropyridine-2-carboxylate

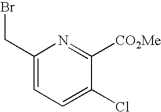

¹H-NMR (CDCl₃) δ 4.01 (3H, s), 4.56 (2H, s), 7.56 (1H, d), 7.81 (1H, d).

### Isopropyl 5-(bromomethyl)-2-chloronicotinate

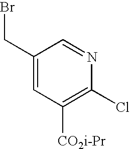

¹H-NMR (CDCl₃) δ 1.41 (6H, d), 4.46 (2H, s), 5.29 (1H, sept), 8.12 (1H, s), 8.49 (1H, s).

### Ethyl 5-(bromomethyl)-2-fluorobenzoate

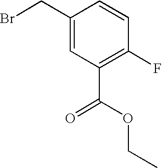

¹H-NMR (CDCl₃) δ: 1.41 (3H, t), 4.40 (2H, q), 4.48 (2H, s), 7.12 (1H, t), 7.51-7.57 (1H, m), 7.95 (1H, dd).

### REFERENCE EXAMPLE 10

### 4-(4-Cyanophenyl)-5-methyl-1H-pyrrole-3-carbonitrile

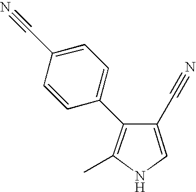

After a solution of 4-[(E)-2-cyanovinyl]benzonitrile (1.54 g), 1-[(1-isocyanoethyl)sulfonyl]-4-methylbenzene (2.09 g) in THF (50 ml) was added to a suspension of sodium hydride (60% oil suspension, 0.48 g) in THF (20 ml) dropwise under ice-cooling, and this was stirred at the same temperature for 2 hours. The reaction mixture was poured into an aqueous saturated sodium chloride solution (100 ml), this was extracted with ethyl acetate, and the ethyl acetate layer was washed with an aqueous saturated sodium chloride solution, dried over magnesium sulfate, and concentrated. The residue was purified by silica gel column chromatography (ethyl acetate-hexane), and crystallized from ether to obtain the desired product (0.60 g) as crystals.

¹H-NMR (CDCl₃) δ 2.37 (3H, s), 7.29 (1H, d), 7.55 (2H, d), 7.72 (2H, d), 8.63 (1H, br s).

According to the same manner as that shown in Reference Example 10, the following compound was obtained.

### Tert-butyl 4-(4-cyanophenyl)-5-methyl-1H-pyrrole-3-carboxylate

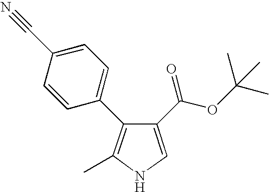

¹H-NMR (CDCl₃) δ1.37 (9H, s), 2.18 (3H, s), 7.34 (1H, d), 7.41 (2H, d), 7.65 (2H, d), 8.39 (1H, s)

### REFERENCE EXAMPLE 11

### 4-(4-Cyanophenyl)-2-formyl-5-methyl-1H-pyrrole-3-carbonitrile

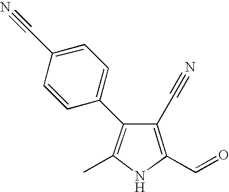

A mixture of 4-(4-cyanophenyl)-5-methyl-1H-pyrrole-3-carbonitrile (2.07 g) and (chloromethylene)dimethylammonium chloride (1.41 g) in DMF (20 ml) was stirred at 80° C. for 2 hours. The reaction mixture was poured into an aqueous saturated sodium chloride solution (100 ml), this was extracted with ethyl acetate, and the ethyl acetate layer was washed with an aqueous saturated sodium chloride solution, dried over magnesium sulfate, and concentrated. The residue was purified by silica gel column chromatography (ethyl acetate-hexane), and crystallized from ethyl acetate to obtain the desired product (1.85 g) as crystals.

¹H-NMR (CDCl₃) δ 2.45 (3H, s), 7.57 (2H, d), 7.78 (2H, d), 9.77 (1H, s), 9.84 (1H, br s).

### REFERENCE EXAMPLE 12

### Ethyl (2E)-3-[3-cyano-4-(4-cyanophenyl)-5-methyl-1H-pyrrole-2-yl]acrylate

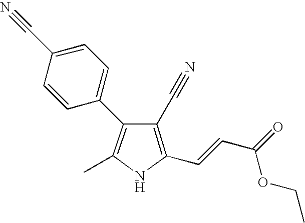

Ethyl diethylphosphonoacetate (0.40 ml) was added to a suspension of sodium hydride (60% oil suspension, 0.65 g) in DMF (30 ml) under ice-cooling. The reaction mixture was stirred for 30 minutes under ice-cooling, 4-(4-cyanophenyl)-2-formyl-5-methyl-1H-pyrrole-3-carbonitrile (0.60 g) was added, and this was stirred at room temperature for 4 hours. The reaction mixture was poured into an aqueous saturated sodium chloride solution, this was extracted with ethyl acetate, and the ethyl acetate layer was dried over magnesium sulfate, and concentrated. The residue was suspended in ether, and this was filtered to obtain the desired product (0.40 g) as a solid.

¹H-NMR (CDCl₃) δ 1.37 (3H, t), 2.42 (3H, s), 4.31 (2H, q), 6.53 (1H, d), 7.56 (2H, d), 7.64 (1H, d), 7.75 (2H, d), 9.76 (1H, br s).

### REFERENCE EXAMPLE 13

### 4-(4-Cyanophenyl)-2-(1,3-dioxolan-2-yl)-5-methyl-1H-pyrrole-3-carbonitrile

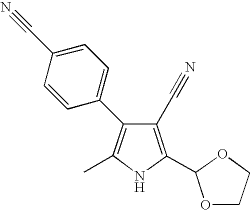

A solution of 4-(4-cyanophenyl)-2-formyl-5-methyl-1H-pyrrole-3-carbonitrile (1.01 g), ethylene glycol (2.5 ml), and p-toluenesulfonic acid monohydrate (9 mg) in toluene (25 ml) was heated to reflux for 2 hours using a Dean-Stark apparatus while dehydrating. The reaction mixture was poured into an aqueous saturated sodium chloride solution (100 ml), this was extracted with ethyl acetate, and ethyl acetate layer was washed with an aqueous saturated sodium chloride solution, dried over magnesium sulfate, and concentrated. The residue was purified by silica gel column chromatography (ethyl acetate-hexane), and crystallized from ethyl acetate to obtain the desired product (0.64 g) as crystals.

¹H-NMR (CDCl₃) δ 2.35 (3H, s), 4.0-4.3 (4H, m), 6.00 (1H, s), 7.53 (2H, d), 7.72 (2H, d), 8.77 (1H, br s).

### REFERENCE EXAMPLE 14

### 4-(4-Cyanophenyl)-2-(hydroxymethyl)-5-methyl-1H-pyrrole-3-carbonitrile

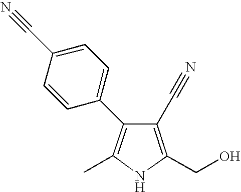

A solution of 4-(4-cyanophenyl)-2-formyl-5-methyl-1H-pyrrole-3-carbonitrile (2.60 g), and sodium borohydride (0.84 g) in THF (40 ml) was heated to reflux at room temperature for 14 hours. The reaction mixture was poured into an aqueous saturated sodium chloride solution (100 ml), this was extracted with ethyl acetate, and the ethyl acetate layer was washed with an aqueous saturated sodium chloride solution, and dried over magnesium sulfate, and concentrated. The residue was purified by silica gel column chromatography (ethyl acetate-hexane), and crystallized from ethyl acetate to obtain the desired product (0.93 g) as crystals.

¹H NMR (DMSO-D₆) δ 2.25 (3H, s), 4.51 (2H, d), 5.49 (1H, br s), 7.58 (2H, d), 7.90 (2H, d), 11.98 (1H, br s)

### REFERENCE EXAMPLE 15

### 4-(4-Cyanophenyl)-2-(methoxymethyl)-5-methyl-1H-pyrrole-3-carbonitrile

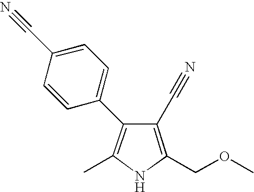

A solution of 4-(4-cyanophenyl)-2-(hydroxymethyl)-5-methyl-1H-pyrrole-3-carbonitrile (0.65 g), and sodium methoxide (4.44 g) in methanol (20 ml) was stirred at 80° C. for 20 hours. The reaction mixture was poured into an aqueous saturated sodium chloride solution (100 ml), this was extracted with ethyl acetate, and the ethyl acetate layer was washed with an aqueous saturated sodium chloride solution, dried over magnesium sulfate, and concentrated. The residue was purified by silica gel column chromatography (ethyl acetate-hexane) to obtain the desired product (42 mg) as crystals.

¹H-NMR (CDCl₃) δ: 2.35 (3H, s), 3.46 (3H, s), 4.59 (2H, s), 7.53 (2H, d), 7.71 (2H, d), 8.66 (1H, br s).

According to the same manner as that shown in Reference Example 15, the following compounds were obtained.

### 4-(4-Cyanophenyl)-2-(ethoxymethyl)-5-methyl-1H-pyrrole-3-carbonitrile

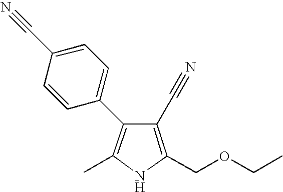

¹H-NMR (CDCl₃) δ 1.28 (3H, t), 2.35 (3H, s), 3.63 (2H, q), 4.63 (2H, s), 7.53 (2H, d), 7.71 (2H, d), 8.74 (1H, br s).

### 4-(4-Cyanophenyl)-5-methyl-2-[(methylthio)methyl]-1H-pyrrole-3-carbonitrile

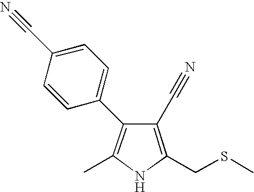

¹H-NMR (CDCl₃) δ 2.15 (3H, s), 2.20 (3H, s), 3.76 (2H, s), 5.32 (2H, s), 7.54 (2H, d), 7.73 (2H, d), 7.94 (1H, br s).

### REFERENCE EXAMPLE 16

### 4-(4-Cyanophenyl)-5-methyl-2-[(methylsulfonyl)methyl]-1H-pyrrole-3-carbonitrile

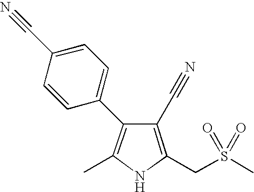

A solution of 4-(4-cyanophenyl)-2-(hydroxymethyl)-5-methyl-1H-pyrrole-3-carbonitrile (0.16 g), and sodium methanesulfinate (0.28 g) in formic acid (1.5 ml) was stirred at room temperature for 20 hours. After an aqueous saturated sodium chloride solution (20 ml) was added to the reaction mixture, this was extracted with ethyl acetate, and the ethyl acetate layer was washed with an aqueous saturated sodium chloride solution, and dried over magnesium sulfate, and concentrated. The residue was crystallized from ether and ethyl acetate to obtain the desired product (0.18 g) as crystals.

¹H NMR (DMSO-D₆) δ 2.30 (3H, s), 3.09 (3H, s), 4.61 (2H, s), 7.62 (2H, d), 7.92 (2H, d), 12.23 (1H, s).

### REFERENCE EXAMPLE 17

### 4-(4-Cyanophenyl)-2-iodo-5-methyl-1H-pyrrole-3-carbonitrile

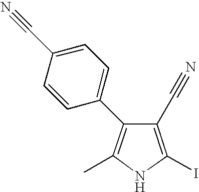

A mixture of 4-(4-cyanophenyl)-5-methyl-1H-pyrrole-3-carbonitrile (1.75 g) and N-iodosuccinic acid imide (2.09 g) in DMF (30 ml) was stirred at room temperature for 2 hours. The reaction mixture was poured into an aqueous saturated sodium chloride solution (100 ml), this was extracted with ethyl acetate, and the ethyl acetate layer was washed with an aqueous saturated sodium chloride solution, dried over magnesium sulfate, and concentrated. The residue was purified by silica gel column chromatography (ethyl acetate-hexane), and crystallized from ethyl acetate to obtain the desired product (2.12 g) as crystals.

¹H-NMR (CDCl₃) δ 2.38 (3H, s), 7.51 (2H, d), 7.72 (2H, d), 8.77 (1H, br s).

### REFERENCE EXAMPLE 18

### Tert-butyl 3-bromo-4-cyano-2,5-dimethyl-1H-pyrrole-1-carboxylate

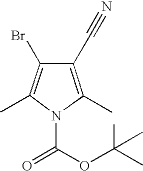

A solution of 3-bromo-4-cyano-2,5-dimethyl-1H-pyrrole (1.94 g), di-tert-butyl dicarbonate (1.61 ml) and 4-dimethylaminopyridine (0.86 g) in acetonitrile (12 ml) was stirred at room temperature for 20 hours. After an aqueous saturated sodium chloride solution (20 ml) was added to the reaction mixture, this was extracted with ethyl acetate, and the ethyl acetate layer was washed with an aqueous saturated sodium chloride solution, dried over magnesium sulfate, and concentrated. The residue was crystallized from ether and ethyl acetate to obtain the desired product (2.76 g) as crystals.

¹H-NMR (CDCl₃) δ 1.62 (9H, s), 2.37 (3H, s), 2.56 (3H, s).

According to the same manner as that shown in Reference Example 18, the following compound was obtained.

### Tert-butyl 3-cyano-4-(4-cyanophenyl)-2-iodo-5-methyl-1H-pyrrole-1-carboxylate

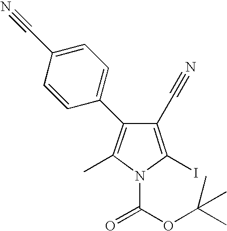

¹H-NMR (CDCl₃) δ 1.67 (9H, s), 2.38 (3H, s), 7.49 (2H, d) 7.73 (2H, d).

### REFERENCE EXAMPLE 19

### 4-(4-Cyanophenyl)-5-methyl-2-(methylthio)-1H-pyrrole-3-carbonitrile

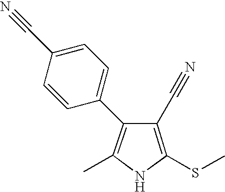

A solution of butyllithium in hexane (1.6 mol/ml, 1.2 ml) was added dropwise to a solution of tert-butyl 3-cyano-4-(4-cyanophenyl)-2-iodo-5-methyl-1H-pyrrole-1-carboxylate (0.69 g) in tetrahydrofuran (10 ml) at −78° C. The reaction mixture was stirred at the same temperature for 30 minutes, dimethyl disulfide (0.17 ml) was added, a temperature was raised to room temperature. After an aqueous saturated sodium chloride solution (20 ml) was added to the reaction mixture, this was extracted with ethyl acetate, and the ethyl acetate layer was washed with an aqueous saturated sodium chloride solution, dried over magnesium sulfate, and concentrated. The residue was purified by silica gel column chromatography (ethyl acetate-hexane), and crystallized from ethyl acetate to obtain the desired product (70 mg) as crystals.

¹H-NMR (CDCl₃) δ 2.35 (3H, s), 2.52 (3H, s), 7.53 (2H, d), 7.72 (2H, d), 8.49 (1H, br s).

### REFERENCE EXAMPLE 20

### [1-(Tert-butoxycarbonyl)-4-cyano-2,5-dimethyl-1H-pyrrole-3-yl]boronic acid

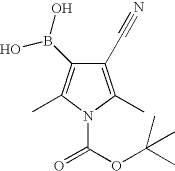

A solution of butyllithium in hexane (1.6 mol/l, 12.5 ml) was added dropwise to a solution of tert-butyl 3-bromo-4-cyano-2,5-dimethyl-1H-pyrrole-1-carboxylate (5.0 g) in tetrahydrofuran (75 ml) at −78° C. The reaction mixture was stirred at the same temperature for 30 minutes, a solution of trimethyl borate (20 ml) in tetrahydrofuran (210 ml) was added, and this was further stirred for 1 hour. After a mixture of water (20 ml) and methanol (20 ml) was added to the reaction mixture, and a temperature was raised to room temperature. After an aqueous saturated sodium chloride solution (100 ml) was added to the reaction mixture, this was extracted with ethyl acetate, and the ethyl acetate layer was washed with an aqueous saturated sodium chloride solution, dried over magnesium sulfate, and concentrated. The residue was crystallized from ether and ethyl acetate to obtain the desired product (4.93 g) as a solid.

¹H-NMR (CDCl₃) δ 1.6-1.7 (1H, br s), 1.64 (9H, s), 2.54 (3H, s), 2.60 (3H, s).

### REFERENCE EXAMPLE 21

### 4-(3-Cyanophenyl)-2,5-dimethyl-1H-pyrrole-3-carbonitrile

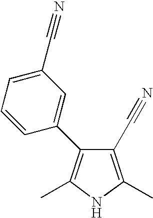

A mixture of 3-bromo-4-cyano-2,5-dimethyl-1H-pyrrole (2.45 g), 3-cyanophenylboronic acid (2.16 g), tetrakis(triphenylphosphine)palladium (0.60 g), anhydrous potassium carbonate (3.90 g), DMF (90 ml) and water (12 ml) was heated at 130° C. for 20 hours, and cooled to room temperature. After an aqueous saturated sodium chloride solution (100 ml) was added to the reaction mixture, this was extracted with ethyl acetate, and the ethyl acetate layer was washed with an aqueous saturated sodium chloride solution, dried over magnesium sulfate, and concentrated. The residue was purified by silica gel column chromatography (ethyl acetate-hexane), and crystallized from ethyl acetate to obtain the desired product (0.91 g) as crystals.

¹H-NMR (CDCl₃) δ 2.29 (3H, s), 2.41 (3H, s), 7.4-7.5 (2H, m), 7.6-7.8 (2H, m), 8.30 (1H, br s).

### REFERENCE EXAMPLE 22

### 4-[4-Cyano-3-(trifluoromethyl)phenyl]-2,5-dimethyl-1H-pyrrole-3-carbonitrile

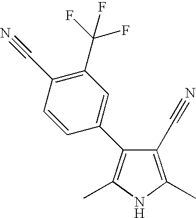

A mixture of [1-(tert-butoxycarbonyl)-4-cyano-2,5-dimethyl-1H-pyrrole-3-yl]boronic acid (6.0 g), 4-bromo-2-(trifluoromethyl)benzonitrile (5.68 g), potassium fluoride (4.62 g), tri t-butylphosphine (0.36 mol/l solution in hexane, 3.2 ml), tris(dibenzylideneacetone)dipalladium (0.42 g), and THF (50 ml) was stirred at room temperature for 20 hours. After an aqueous saturated sodium chloride solution (100 ml) was added to the reaction mixture, this was extracted with ethyl acetate, and the ethyl acetate layer was washed with an aqueous saturated sodium chloride solution, dried over magnesium sulfate, and concentrated. The residue was purified by silica gel column chromatography (ethyl acetate-hexane), and crystallized from ethyl acetate to obtain the desired product (3.39 g) as crystals.

¹H-NMR (CDCl₃) δ 2.34 (3H, s), 2.46 (3H, s), 7.7-7.8 (2H, m), 7.88 (1H, d), 8.25 (1H, br s).

According to the same manner as that shown in Reference Example 22, the following compound was obtained.

### 4-(4-Cyano-2-fluorophenyl)-2,5-dimethyl-1H-pyrrole-3-carbonitrile

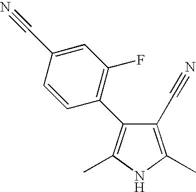

¹H-NMR (CDCl₃) δ2.30 (3H, s), 2.44 (3H, s), 7.20-7.30 (1H, m), 7.30-7.50 (2H, m), 8.24 (1H, br s).

### REFERENCE EXAMPLE 23

### Methyl 4-(4-cyanophenyl)-5-methyl-2-(trifluoromethyl)-1H-pyrrole-3-carboxylate

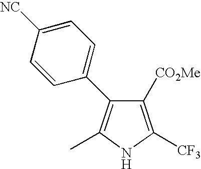

Methyl trifluoroacetoacetate (21.4 g) To a mixture of potassium hydroxide (2.39 g) and THF (75 ml) was added dropwise over 5 minutes under ice-cooling, and this was stirred for 45 minutes. 4-[(1E)-2-nitroprop-1-en-1-yl]benzonitrile (8.0 g) was added dropwise to this reaction mixture for 20 minutes, this was stirred at room temperature for 48 hours, and water and ethyl acetate were added to distribute therebetween. The organic layer was dried over magnesium sulfate, and concentrated. A mixture of the residue, water (2.24 ml), methanol (22.4 ml) and concentrated hydrochloric acid (2.24 ml) was heated to reflux for 1 hour, and cooled to room temperature, and water and ethyl acetate were added to distribute therebetween. The aqueous layer was extracted with ethyl acetate, and the organic layers were combined, washed with an aqueous saturated sodium dicarbonate solution and an aqueous sodium chloride solution, dried (magnesium sulfate) and concentrated. The residue was crystallized from diisopropyl ether to obtain the desired product (3.45 g) as crystals.

¹H-NMR (CDCl₃) δ: 2.20 (3H, s), 3.70 (3H, s), 7.36 (2H, d), 7.66 (2H, d), 8.85 (1H, s).

### REFERENCE EXAMPLE 24

### Ethyl 2-chloro-5-{[3-cyano-4-(4-cyanophenyl)-2,5-dimethyl-1H-pyrrol-1-yl]methyl}nicotinate

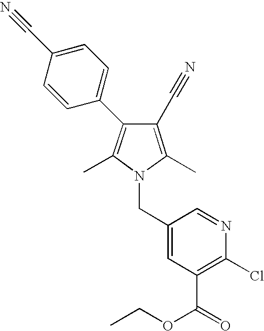

Sodium hydride (60% oil suspension, 0.65 g) was added to a solution of 4-(4-cyanophenyl)-2,5-dimethyl-1H-pyrrole-3-carbonitrile (3.0 g) in DMF (30 ml) under ice-cooling. The reaction mixture was stirred at room temperature for 30 minutes, ethyl 5-(bromomethyl)-2-chloronicotinate (4.17 g) was added, and the mixture was stirred at room temperature for 4 hours. The reaction mixture was poured into an aqueous saturated sodium chloride solution, this was extracted with ethyl acetate, and the ethyl acetate layer was dried over magnesium sulfate, and concentrated. The residue was purified by silica gel column chromatography (ethyl acetate-hexane), and crystallized from ethyl acetate to obtain the desired product (3.65 g) as crystals.

¹H-NMR (200 MHz, CDCl₃) δ 1.41 (3H, t), 2.21 (3H, s), 2.40 (3H, s), 4.43 (2H, q), 5.16 (2H, s), 7.51 (2H, d), 7.71 (1H, s), 7.73 (2H, d), 8.13 (1H, d).

According to the same manner as that shown in Reference Example 24, the following compounds were obtained.

### Ethyl 2-chloro-5-{[3-cyano-4-(4-cyanophenyl)-2-ethyl-5-methyl-1H-pyrrol-1-yl]methyl}nicotinate

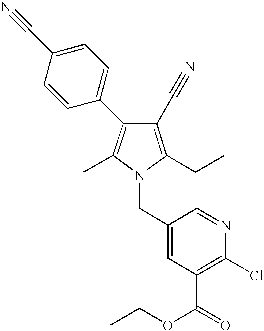

¹H-NMR (CDCl₃) δ 1.25 (3H, t), 1.41 (3H, t), 2.17 (3H, s), 2.79 (2H, q), 4.43 (2H, q), 5.18 (2H, s); 7.52 (2H, d), 7.7-7.8 (3H, m), 8.10 (1H, d).

### Ethyl 2-chloro-5-{[3-cyano-4-(4-cyanophenyl)-5-methyl-2-propyl-1H-pyrrol-1-yl]methyl}nicotinate

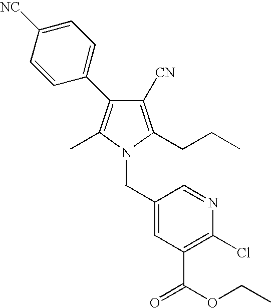

¹H-NMR (CDCl₃) δ: 1.00 (3H, t), 1.40 (3H, t), 1.56-1.68 (2H, m), 2.16 (3H, s), 2.73 (2H, t), 4.42 (2H, q), 5.16 (2H, s), 7.48-7.52 (2H, m), 7.68-7.73 (3H, m), 8.08 (1H, d)

### Ethyl 2-chloro-5-{[3-cyano-4-(4-cyanophenyl)-2-isopropyl-5-methyl-1H-pyrrol-1-yl]methyl}nicotinate

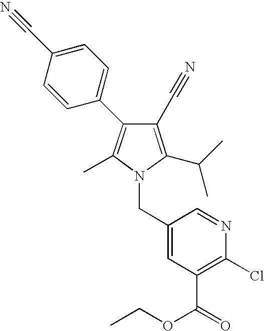

¹H-NMR (CDCl₃) δ 1.3-1.5 (9H, m), 2.17 (3H, s), 2.98 (1H, sept), 4.43 (2H, q), 5.20 (2H, s), 7.51 (2H, d), 7.7-7.8 (3H, m), 8.11 (1H, d).

### Ethyl 2-chloro-5-{[3-cyano-4-(4-cyanophenyl)-2-cyclopropyl-5-methyl-1H-pyrrol-1-yl]methyl}nicotinate

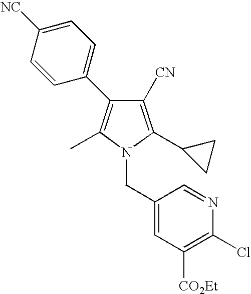

¹H-NMR (QDCl₃) δ: 0.97-1.06 (4H, m), 1.41 (3H, t), 1.59-1.68 (1H, m), 2.18 (3H, s), 4.43 (2H, q), 5.34 (2H, s), 7.48 (2H, d), 7.70 (2H, d), 7.75 (1H, d), 8.14 (1H, d)

### Isopropyl 2-chloro-5-({3-cyano-4-[4-cyano-3-(trifluoromethyl)phenyl]-2,5-dimethyl-1H-pyrrol-1-yl}methyl)nicotinate

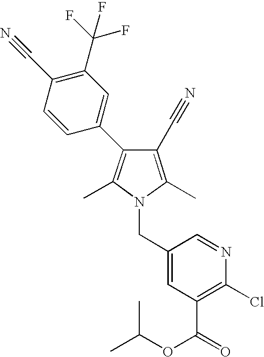

¹H-NMR (CDCl₃) δ: 1.40 (6H, d), 2.24 (3H, s), 2.42 (3H, s), 5.17 (2H, s), 5.29 (1H, sept), 7.7-7.8 (3H, m), 7.91 (1H, d), 8.10 (1H, d).

### Ethyl 2-chloro-5-{[3-cyano-4-(3-cyanophenyl)-2,5-dimethyl-1H-pyrrol-1-yl]methyl}nicotinate

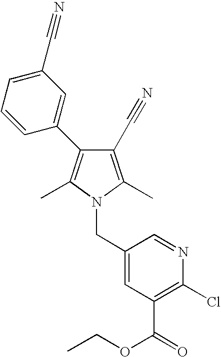

¹H-NMR (CDCl₃) δ 1.41 (3H, t), 2.0 (3H, s), 2.40 (3H, s), 4.43 (2H, q), 5.16 (2H, s), 7.57 (1H, d), 7.6-7.7 (4H, m), 8.14 (1H, d).

### Ethyl 2-chloro-5-{[3-cyano-4-(4-cyanophenyl)-5-ethyl-2-methyl-1H-pyrrol-1-yl]methyl}nicotinate

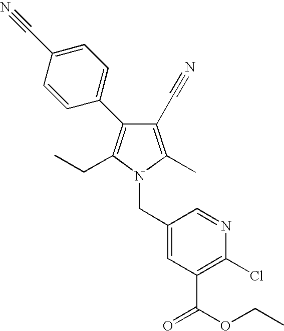

¹H-NMR (CDCl₃) δ: 1.12 (3H, t), 1.41 (3H, t), 2.34 (3H, s), 2.58 (2H, q), 4.42 (2H, q), 5.17 (2H, s), 7.51 (2H, d), 7.67-7.73 (3H, m), 8.06 (1H, d).

### Ethyl 2-chloro-5-{[3-(4-cyanophenyl)-4-(methoxycarbonyl)-2-methyl-5-(trifluoromethyl)-1H-pyrrol-1-yl]methyl}nicotinate

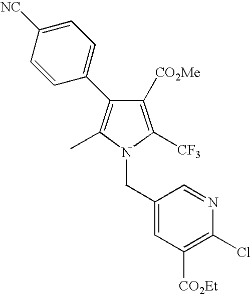

¹H-NMR (CDCl₃) δ: 1.41 (3H, t), 2.11 (3H, s), 3.72 (3H, s), 4.43 (2H, q), 5.31 (2H, s), 7.36 (2H, d), 7.69 (2H, d), 7.80 (1H, d), 8.14 (1H, d).

### Methyl 5-chloro-2-{[3-cyano-4-(4-cyanophenyl)-2,5-dimethyl-1H-pyrrol-1-yl]methyl}nicotinate

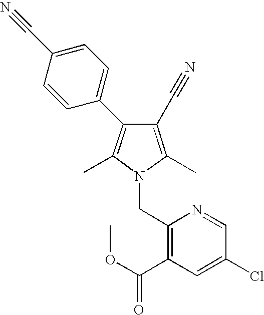

¹H-NMR (CDCl₃) δ 2.19 (3H, s), 2.30 (3H, s), 4.02 (3H, s), 5.58 (2H, s), 7.54 (2H, d), 7.70 (2H, d), 8.35 (1H, d), 8.59 (1H, d).

### Methyl 5-chloro-2-{[3-cyano-4-(4-cyanophenyl)-2,5-dimethyl-1H-pyrrol-1-yl]methyl}isonicotinate

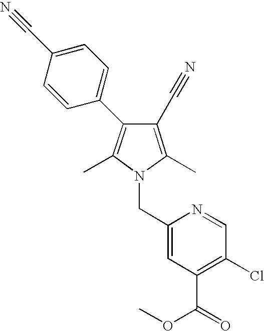

¹H-NMR (CDCl₃) δ 2.17 (3H, s), 2.35 (3H, s), 4.03 (3H, s), 5.51 (2H, s), 7.42 (1H, s), 7.53 (2H, d), 7.73 (2H, d), 7.95 (1H, s).

### Methyl 3-chloro-6-{[3-cyano-4-(4-cyanophenyl)-2,5-dimethyl-1H-pyrrol-1-yl]methyl}pyridine-2-carboxylate

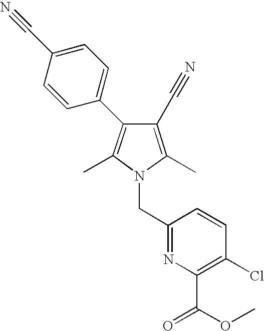

¹H-NMR (CDCl₃) δ 2.21 (3H, s), 2.39 (3H, s), 4.04 (3H, s), 5.23 (2H, s), 6.69 (1H, d), 7.52 (2H, d), 7.7-7.8 (3H, m).

### Ethyl 2-chloro-5-{[4-cyano-3-(4-cyanophenyl)-2-methyl-1H-pyrrol-1-yl]methyl}nicotinate

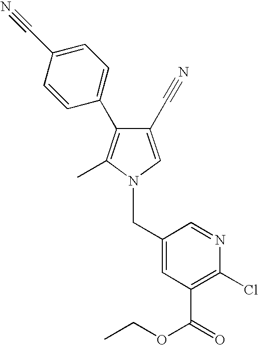

¹H-NMR (CDCl₃) δ 1.42 (3H, t), 2.22 (3H, s), 4.44 (2H, q), 5.16 (2H, s), 7.28 (1H, s), 7.51 (2H, d), 7.74 (2H, d), 7.85 (1H, d), 8.29 (1H, d).

### Ethyl 2-chloro-5-{[3-cyano-4-(4-cyanophenyl)-5-methyl-2-(methylthio)-1H-pyrrol-1-yl]methyl}nicotinate

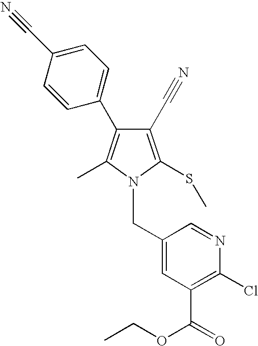

¹H-NMR (CDCl₃) δ 1.42 (3H, t), 2.24 (3H, s), 2.42 (3H, s), 4.43 (2H, q), 5.42 (2H, s), 7.51 (2H, d), 7.74 (2H, d), 7.82 (1H, d), 8.20 (1H, d).

### Ethyl-2-chloro-5-{[3-cyano-4-(4-cyanophenyl)-2-formyl-5-methyl-1H-pyrrol-1-yl]methyl}nicotinate

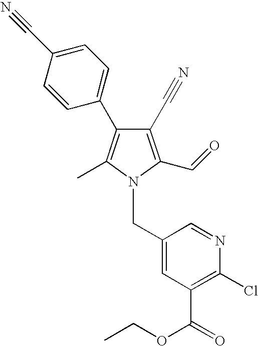

¹H-NMR (CDCl₃) δ 1.42 (3H, t), 2.35 (3H, s), 4.43 (2H, q), 5.74 (2H, s), 7.53 (2H, d), 7.80 (2H, d), 7.88 (1H, d), 8.26 (1H, d), 9.89 (1H, s).

### Ethyl 2-chloro-5-{[3-cyano-4-(4-cyanophenyl)-2-iodo-5-methyl-1H-pyrrol-1-yl]methyl}nicotinate

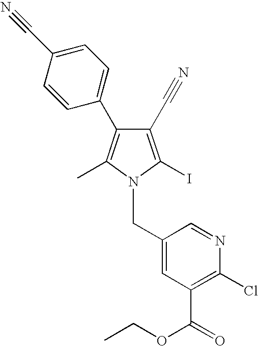

¹H-NMR (CDCl₃) δ 1.42 (3H, t), 2.22 (3H, s), 4.43 (2H, q), 5.16 (2H, s), 7.51 (2H, d), 7.73 (2H, d), 7.85 (1H, d), 8.28 (1H, d).

### Ethyl 2-chloro-5-{[3-cyano-4-(4-cyanophenyl)-2-(1,3-dioxolan-2-yl)-5-methyl-1H-pyrrol-1-yl]methyl}nicotinate

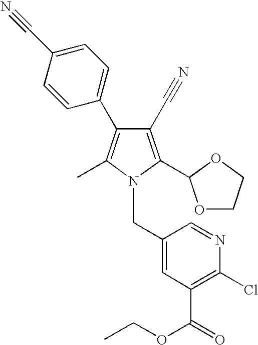

¹H-NMR (CDCl₃) δ 1.26 (3H, t), 2.13 (3H, s), 4.0-4.2 (4H, m), 4.43 (2H, q), 5.32 (2H, s), 6.04 (1H, s), 7.49 (2H, d), 7.73 (2H, d), 7.91 (1H, d), 8.25 (1H, d).

### Ethyl 2-chloro-5-{[3-cyano-4-(4-cyanophenyl)-2-(methoxymethyl)-5-methyl-1H-pyrrol-1-yl]methyl}nicotinate

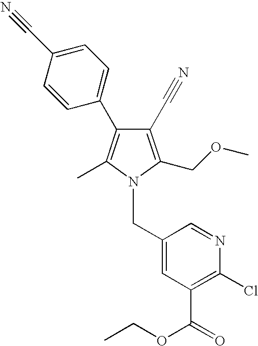

¹H-NMR (CDCl₃) δ 1.41 (3H, t), 2.19 (3H, s), 3.37 (3H, s), 4.42 (2H, q), 4.55 (2H, s), 5.27 (2H, s), 7.50 (2H, d), 7.73 (2H, d), 7.85 (1H, d), 8.20 (1H, d).

### Ethyl 2-chloro-5-{[3-cyano-4-(4-cyanophenyl)-2-(ethoxymethyl)-5-methyl-1H-pyrrol-1-yl]methyl}nicotinate

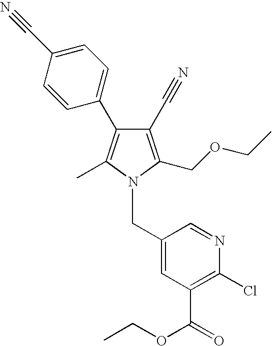

¹H-NMR (CDCl₃) δ 1.14 (3H, t), 1.41 (3H, t), 2.18 (3H, s), 3.56 (2H, q), 4.42 (2H, q), 4.59 (2H, s), 5.28 (2H, s), 7.50 (2H, d), 7.73 (2H, d), 7.85 (1H, d), 8.21 (1H, d).

### Ethyl 2-chloro-5-({3-cyano-4-(4-cyanophenyl)-5-methyl-2-[(methylthio)methyl]-1H-pyrrol-1-yl}methyl)nicotinate

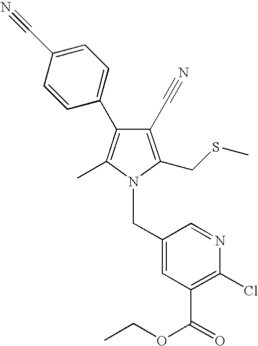

¹H-NMR (CDCl₃) δ 1.41 (3H, t), 2.14 (3H, s), 2.19 (3H, s), 3.77 (2H, s), 4.43 (2H, q), 5.33 (2H, s), 7.52 (2H, d), 7.7-7.8 (3H, m), 8.13 (1H, d).

### Ethyl 2-chloro-5-({3-cyano-4-(4-cyanophenyl)-5-methyl-2-[(methylsulfonyl)methyl]-1H-pyrrol-1-yl}methyl)nicotinate

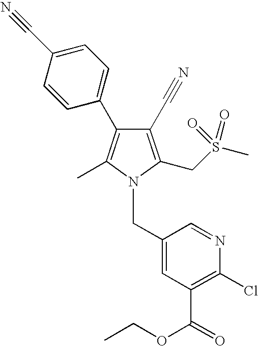

¹H-NMR (CDCl₃) δ 1.42 (3H, t), 2.25 (3H, s), 3.05 (3H, s), 4.4-4.5 (4H, m), 5.49 (2H, s), 7.53 (2H, d), 7.7-7.8 (3H, m), 8.03 (1H, d).

### Methyl 2-chloro-5-{[3-cyano-4-(3-cyanophenyl)-2,5-dimethyl-1H-pyrrol-1-yl]methyl}benzoate

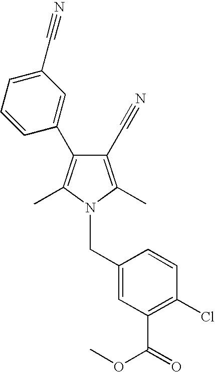

¹H-NMR (CDCl₃) δ 2.17 (3H, s), 2.37 (3H, s), 3.94 (3H, s), 5.11 (2H, s), 6.89 (1H, dd), 7.4-7.6 (2H, m), 7.6-7.7 (4H, m).

### Methyl 2-chloro-5-{[3-cyano-4-(4-cyanophenyl)-2,5-dimethyl-1H-pyrrol-1-yl]methyl}benzoate

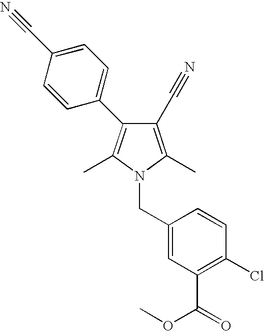

¹H-NMR (200 MHz, CDCl₃) δ 2.19 (3H, s), 2.38 (3H, s), 3.94 (3H, s), 5.11 (2H, s), 6.89 (1H, dd), 7.4-7.5 (4H, m), 7.72 (2H, d).

### Ethyl 2-chloro-5-({3-cyano-4-(4-cyanophenyl)-2-[(1E)-3-ethoxy-3-oxoprop-1-en-1-yl]-5-methyl-1H-pyrrol-1-yl}methyl)nicotinate

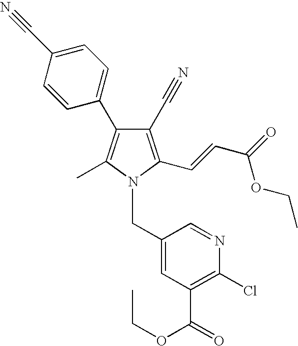

¹H-NMR (CDCl₃) δ 1.32 (3H, t), 1.41 (3H, t), 2.27 (3H, s), 4.24 (2H, q), 4.43 (2H, q), 5.32 (2H, s), 7.4-7.6 (3H, m) 7.7-7.9 (3H, m), 8.14 (2H, d).

### Ethyl 2-chloro-5-{[3-(4-cyanophenyl)-2,5-dimethyl-1H-pyrrol-1-yl]methyl}nicotinate

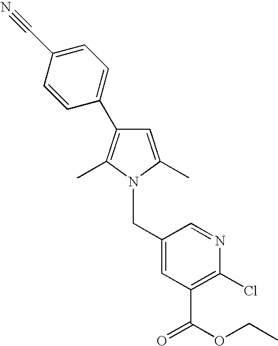

¹H NMR (CDCl₃) δ 1.38 (3H, t), 2.21 (3H, s), 2.28 (3H, s), 4.39 (2H, d), 5.14 (2H, s), 6.14 (1H, s), 7.45 (2H, d), 7.62 (2H, d), 7.72 (1H, d), 8.07 (1H, d).

### Ethyl 2-chloro-5-{[3-(4-cyanophenyl)-5-ethyl-2-methyl-1H-pyrrol-1-yl]methyl}nicotinate

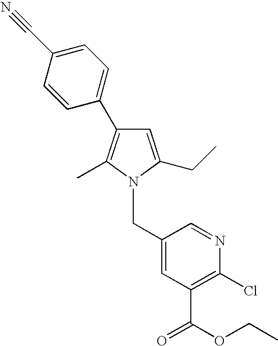

¹H NMR (CDCl₃) δ 1.26 (3H, t), 1.39 (3H, t), 2.27 (3H, s), 2.50 (2H, q), 4.40 (2H, q), 5.12 (2H, s), 6.15 (1H, s), 7.44-7.48 (2H, m), 7.62-7.65 (2H, m), 7.65-7.69 (1H, m), 8.02-8.04 (1H, m).

### Ethyl 2-chloro-5-{[3-(4-cyanophenyl)-5-methyl-2-(trifluoromethyl)-1H-pyrrol-1-yl]methyl}nicotinate

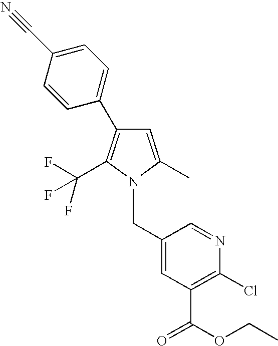

¹H NMR (CDCl₃) : 1.41 (3H, t), 2.19 (3H, s), 4.42 (2H, q), 5.27 (2H, s), 6.08 (1H, s), 7.45 (2H, d), 7.66 (2H, d), 7.77 (1H, d), 8.07 (1H, d).

### Ethyl 2-chloro-5-{[3-(4-cyanophenyl)-2-formyl-5-methyl-1H-pyrrol-1-yl]methyl}nicotinate

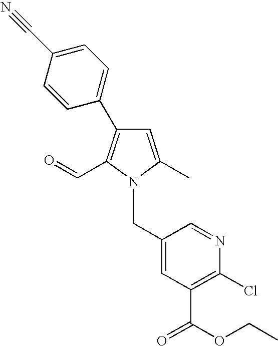

¹H NMR (CDCl₃) δ: 1.40 (3H, t), 2.33 (3H, s), 4.41 (2H, q), 5.70 (2H, s), 6.25 (1H, s), 7.52 (2H, d), 7.72 (2H, d), 7.88 (1H, d), 8.19 (1H, d).

### Ethyl 2-chloro-5-{[4-(4-cyanophenyl)-2-methyl-1H-pyrrol-1-yl]methyl}nicotinate

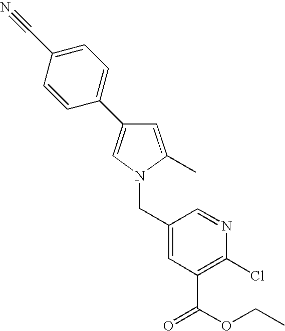

¹H NMR (CDCl₃): 1.38 (3H, t), 2.20 (3H, s), 4.40 (2H, q), 5.09 (2H, s), 6.31 (1H, s), 6.99 (1H, d), 7.05-7.59 (4H, m), 7.79 (1H, d), 8.22 (1H, d).

### Ethyl 2-chloro-5-{[2-cyano-3-(4-cyanophenyl)-5-methyl-1H-pyrrol-1-yl]methyl}nicotinate

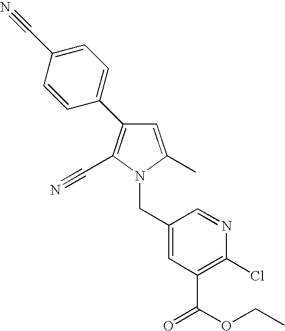

¹H NMR (CDCl₃): 1.40 (3H, t), 2.29 (3H, s), 4.41 (2H, q), 5.29 (2H, s), 6.35 (1H, s), 7.69 (2H, d), 7.76 (2H, d), 7.89 (1H, dd), 8.24-8.25 (1H, m).

### Ethyl 2-chloro-5-{[3-cyano-4-(4-cyano-2-fluorophenyl)-2,5-dimethyl-1H-pyrrol-1-yl]methyl}nicotinate

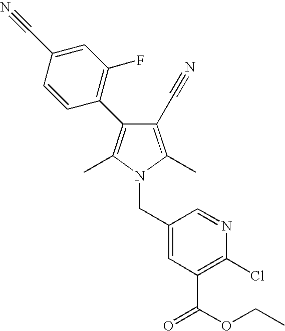

¹H NMR (CDCl₃) δ1.42 (3H, t) 2.21 (3H, s) 2.41 (3H, s) 4.44 (2H, q) 5.16 (2H, s) 7.30-7.40 (2H, m) 7.40-7.50 (1H, m) 7.71 (1H, d) 8.14 (1H, d).

### Ethyl 5-{[3-(tert-butoxycarbonyl)-2-cyano-4-(4-cyanophenyl)-5-methyl-1H-pyrrol-1-yl]methyl}-2-chloronicotinate

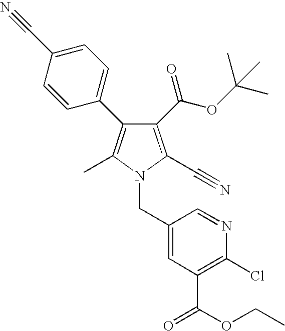

According to the same manner as that shown in Reference Example 24, an oily material (0.52 g) containing ethyl 5-{[3-(tert-butoxycarbonyl)-2-cyano-4-(4-cyanophenyl)-5-methyl-1H-pyrrol-1-yl]methyl}-2-chloronicotinate was obtained. This oily material was used in the subsequent reaction without isolation and purification.

Ethyl 5-{[3-(tert-butoxycarbonyl)-4-(4-cyanophenyl)-5-methyl-2-(trifluoromethyl)-1H-pyrrol-1-yl]methyl}-2-chloronicotinate

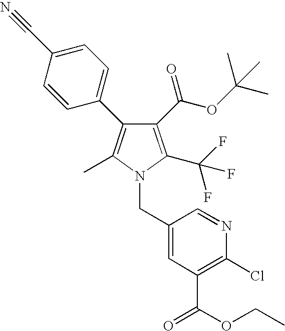

According to the same manner as that shown in Reference Example 24, an oily material (0.25 g) containing ethyl 5-{[3-(tert-butoxycarbonyl)-4-(4-cyanophenyl)-5-methyl-2-(trifluoromethyl)-1H-pyrrol-1-yl]methyl}-2-chloronicotinate was obtained. This oily material was used in the subsequent reaction without isolation and purification.

### REFERENCE EXAMPLE 25

### Ethyl 2-chloro-5-{[3-cyano-4-(4-cyanophenyl)-5-methyl-2-(1,3-oxazol-5-yl)-1H-pyrrol-1-yl]methyl}nicotinate

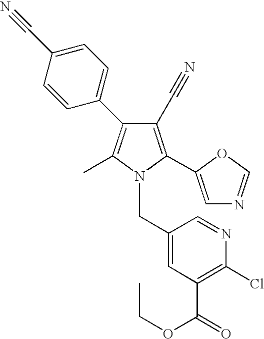

A mixture of ethyl 2-choro-5-{[3-cyano-4-(4-cyanophenyl)-2-formyl-5-methyl-1H-pyrrol-1-yl]methyl}nicotinate (0.31 g), toluenesulfonylmethyl isocyanide (0.15 g), potassium carbonate (0.11 g) and ethanol (5 ml) was stirred at 80° C. for 2 hours. The reaction mixture was poured into an aqueous saturated sodium chloride solution (5 ml), this was extracted with ethyl acetate, and the ethyl acetate layer was washed with an aqueous saturated sodium chloride solution, dried over magnesium sulfate, and concentrated. The residue was purified by silica gel column chromatography (ethyl acetate-hexane) to obtain the desired product (0.22 g) as an oil.

¹H-NMR (CDCl₃) δ 1.41 (3H, t), 2.28 (3H, s), 4.43 (2H, q), 5.37 (2H, s), 7.4-7.6 (3H, m), 7.77 (2H, d), 7.81 (1H, d), 7.97 (1H, d), 8.21 (1H, d).

### REFERENCE EXAMPLE 26

### Ethyl 2-chloro-5-({3-cyano-4-(4-cyanophenyl)-2-[(dimethylamino)methyl]-5-methyl-1H-pyrrol-1-yl}methyl)nicotinate

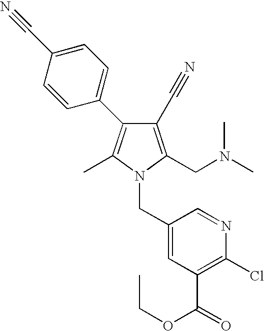

A mixture of ethyl 2-chloro-5-{[4-cyano-3-(4-cyanophenyl)-2-methyl-1H-pyrrol-1-yl]methyl}nicotinate (0.41 g), N,N-dimethylmethyleneammonium iodide (0.22 g) and DMF (5 ml) was stirred at 120° C. for 14 hours. The reaction mixture was poured into an aqueous saturated sodium chloride solution (5 ml), this was extracted with ethyl acetate, and the ethyl acetate layer was washed with an aqueous saturated sodium chloride solution, dried over magnesium sulfate, and concentrated. The residue was purified by silica gel column chromatography (ethyl acetate-hexane) to obtain the desired product (0.14 g) as crystals.

¹H-NMR (CDCl₃) δ 1.40 (3H, t), 2.18 (3H, s), 2.25 (6H, s), 3.51 (2H, s), 4.42 (2H, q), 5.37 (2H, s), 7.51 (2H, d), 7.72 (2H, d), 7.85 (1H, d), 8.21 (1H, d).

### REFERENCE EXAMPLE 27

### Ethyl 2-chloro-5-{[3-cyano-4-(4-cyanophenyl)-5-methyl-2-(methylsulfonyl)-1H-pyrrol-1-yl]methyl}nicotinate

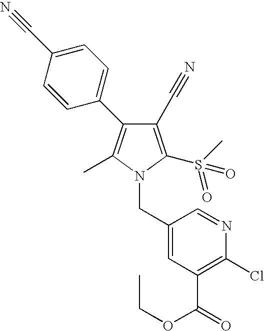

A mixture of ethyl 2-chloro-5-{[3-cyano-4-(4-cyanophenyl)-2-iodo-6-methyl-1H-pyrrole-1-yl]methyl}nicotinate (0.27 g), copper (I) iodide (0.18 g), sodium methanesulfinate (0.33 g), and hexamethylphosphoric acid triamide (6 ml) was stirred at 80° C. for 1 hour. The reaction mixture was poured into an aqueous saturated sodium chloride solution (5 ml), this was extracted with ethyl acetate, and the ethyl acetate layer was washed with an aqueous saturated sodium chloride solution, dried over magnesium sulfate, and concentrated. The residue was purified by silica gel column chromatography (ethyl acetate-hexane), and crystallized from ethyl acetate to obtain the desired product (0.14 g) as crystals.

¹H-NMR (200 MHz, CDCl₃) δ 1.42 (3H, t), 2.28 (3H, s), 3.18 (3H, s), 4.43 (2H, q), 5.68 (2H, s), 7.51 (2H, d), 7.79 (2H, d), 7.91 (1H, d), 8.22 (1H, d).

### REFERENCE EXAMPLE 28

### Ethyl 2-chloro-5-{[3-cyano-4-(4-cyanophenyl)-5-methyl-2-(trifluoromethyl)-1H-pyrrol-1-yl]methyl}nicotinate

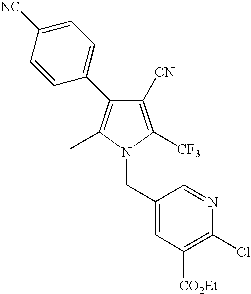

A mixture of ethyl 2-chloro-5-{[3-cyano-4-(4-cyanophenyl)-2-iodo-5-methyl-1H-pyrrole-1-yl]methyl}nicotinate (259 mg), methyl difluoro(fluorosulfonyl)acetate (411 μl), copper iodide (102 mg) and DMF (3.5 ml) was heated at 100° C. for 1 hour under the argon atmosphere. The reaction mixture was cooled to room temperature, poured into water, and distributed with ethyl acetate. The organic layer was washed with water and an aqueous saturated sodium chloride solution, dried (magnesium sulfate), and concentrated. The residue was purified by silica gel column chromatography (ethyl acetate-hexane), and crystallized from ethyl acetate-diisopropyl ether to obtain the desired product (145 mg) as crystals.

¹H-NMR (CDCl₃) δ: 1.41 (3H, t), 2.20 (3H, s), 4.43 (2H, q), 5.33 (2H, s), 7.49 (2H, d), 7.75-7.78 (1H, m), 7.76 (2H, d), 8.13 (1H, d).

### REFERENCE EXAMPLE 29

### Ethyl 2-chloro-5-{[3-cyano-4-(4-cyanophenyl)-5-methyl-2-(2,2,2-trifluoro-1-hydroxyethyl)-1H-pyrrol-1-yl]methyl}nicotinate

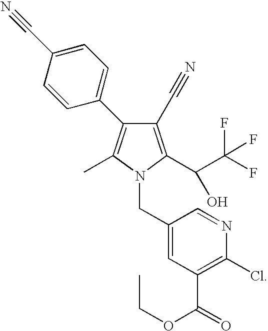

A 1N solution (0.3 ml) of tetrabutylammonium fluoride in THF was added dropwise to a solution of ethyl 2-chloro-5-{[3-cyano-4-(4-cyanophenyl)-2-formyl-5-methyl-1H-pyrrol-1-yl]methyl}nicotinate (0.10 g), and trimethyl(trifluoromethyl)silane (0.035 ml) in THF (3 ml) at room temperature, and this was stirred for 2 hours. The reaction mixture was poured into an aqueous saturated sodium chloride solution (10 ml), this was extracted with ethyl acetate, and the ethyl acetate layer was washed with an aqueous saturated sodium chloride solution, dried over magnesium sulfate, and concentrated. The residue was purified by silica gel column chromatography (ethyl acetate-hexane) to obtain the desired product (51 mg) as an oil.

¹H-NMR (CDCl₃) δ 1.40 (3H, t), 2.12 (3H, s), 4.41 (2H, q), 5.0-5.2 (2H, m), 5.19 (1H, d), 5.76 (1H, d), 7.47 (2H, d), 7.72 (2H, d), 7.84 (1H, d), 8.10 (1H, d).

### REFERENCE EXAMPLE 30

### 1-{[5-({[Tert-butyl(dimethyl)silyl]oxy}methyl)-6-chloropyridin-3-yl]methyl}-4-(4-cyanophenyl)-2-formyl-5-methyl-1H-pyrrole-3-carbonitrile

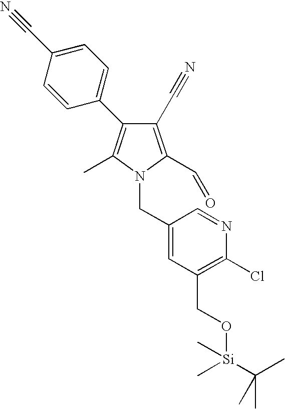

A mixture of 1-{[6-chloro-5-(hydroxymethyl)pyridin-3-yl]methyl}-4-(4-cyanophenyl)-2-formyl-5-methyl-1H-pyrrole-3-carbonitrile (Compound 40, 119 mg), t-butyldimethylsilyl chloride (55 mg), imidazole (51 mg) and DMF (5 ml) was stirred at room temperature for 6 hours. The reaction mixture was added to water (10 ml), this was extracted with ethyl acetate, and the ethyl acetate layer was washed with an aqueous saturated sodium chloride solution, dried over magnesium sulfate, and concentrated. The residue was purified by silica gel column chromatography (ethyl acetate-hexane) to obtain the desired product (144 mg) as a solid.

¹H-NMR (CDCl₃) δ 0.09 (6H, s), 0.90 (9H, s), 2.31 (3H, s), 4.68 (2H, s), 5.76 (2H, s), 7.4-7.6 (3H, m), 7.79 (2H, d), 8.15 (1H, d), 9.89 (1H, s).

### REFERENCE EXAMPLE 31

### 1-{[5-({[Tert-butyl(dimethyl)silyl]oxy}methyl)-6-chloropyridin-3-yl]methyl}-4-(4-cyanophenyl)-2-(1-hydroxyethyl)-5-methyl-1H-pyrrole-3-carbonitrile

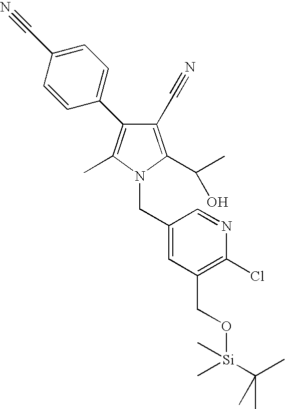

A 3N solution (0.19 ml) of methylmagnesium bromide in THF was added dropwise to a solution of 1-{[5-({[tert-butyl(dimethyl)silyl]oxy}methyl)-6-chloropyridin-3-yl]methyl}-4-(4-cyanophenyl)-2-formyl-5-methyl-1H-pyrrole-3-carbonitrile (0.14 g) in THF (3 ml) under ice-cooling, and this was stirred for 20 minutes under ice-cooling. The reaction mixture was poured into an aqueous saturated sodium chloride solution (10 ml), this was extracted with ethyl acetate, and the ethyl acetate layer was washed with an aqueous saturated sodium chloride solution, dried over magnesium sulfate, and concentrated. The residue was purified by silica gel column chromatography (ethyl acetate-hexane) to obtain the desired product (0.14 g) as a solid.

¹H-NMR (CDCl₃) δ 0.08 (6H, s), 0.87 (9H, s), 1.65 (3H, d), 1.5-1.7 (1H, br s), 2.12 (3H, s), 4.70 (2H, s), 5.22 (1H, q), 5.46 (2H, s), 7.42 (1H, d), 7.48 (2H, d), 7.72 (2H, d), 8.04 (1H, d).

### REFERENCE EXAMPLE 32

### 2-Acetyl-1-{[5-({[tert-butyl(dimethyl)silyl]oxy}methyl)-6-chloropyridin-3-yl]methyl}-4-(4-cyanophenyl)-5-methyl-1H-pyrrole-3-carbonitrile

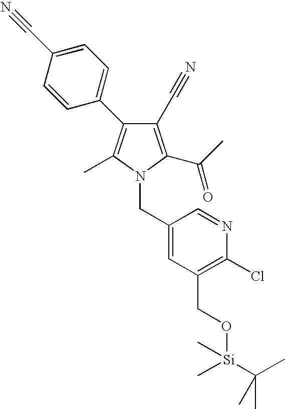

A solution of 1-{[5-({[tert-butyl(dimethyl)silyl]oxy}methyl)-6-chloropyridin-3-yl]methyl}-4-(4-cyanophenyl)-2-(1-hydroxyethyl)-5-methyl-1H-pyrrole-3-carbonitrile (0.14 g) and manganese dioxide (4.2 g) in dichloromethane (5 ml) was stirred at 50° C. for 3 hours. The insolubles were filtered, and the reaction mixture was washed with an aqueous saturated sodium chloride solution, dried over magnesium sulfate, and concentrated. The residue was purified by silica gel column chromatography (ethyl acetate-hexane) to obtain the desired product (90 mg) as an oil.

¹H-NMR (CDCl₃) δ 0.08 (6H, m), 0.89 (9H, m), 2.25 (3H, s), 2.75 (3H, s), 4.68 (2H, s), 5.73 (2H, s), 7.44 (1H, d), 7.50 (2H, d), 7.78 (2H, d), 8.10 (1H, d).

### REFERENCE EXAMPLE 33

### Ethyl 2-chloro-5-{[3-(4-cyanophenyl)-5-cyclopropyl-2-methyl-1H-pyrrol-1-yl]methyl}nicotinate

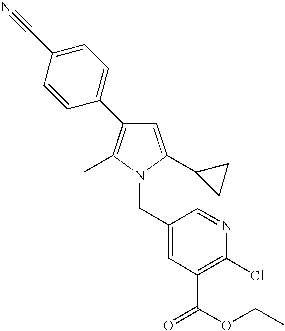

To a solution of sodium hydride (60% suspension in oil, 87 mg) in DMF (8 ml) was added 4-(5-cyclopropyl-2-methyl-1H-pyrrol-3-yl)benzonitrile (400 mg) with ice-cooling. The reaction mixture was stirred at room temperature for 30 minutes, a solution of ethyl 5-(bromomethyl)-2-chloronicotinate (732 mg) in DMF (2 ml) was added thereto, and the mixture was stirred at room temperature for 0.5 hours. The reaction mixture was poured into a saturated sodium chloride solution, the mixture was extracted with ethyl acetate, and the ethyl acetate layer was dried over magnesium sulfate and concentrated. The residue was purified by silica gel column chromatography (ethyl acetate-hexane) and crystallized from diisopropyl ether to obtain ethyl 2-chloro-5-{[4-(4-cyanophenyl)-2-cyclopropyl-5-methyl-1H-pyrrol-3-yl]methyl}nicotinate (96 mg) as crystals, thereby obtaining a mixture of this compound and the desired product (263 mg) as an oily material. This material was used in the subsequent reaction without further purification.

### REFERENCE EXAMPLE 34

### Tert-butyl 4-(4-cyanophenyl)-2-methyl-1H-pyrrole-3-carboxylate

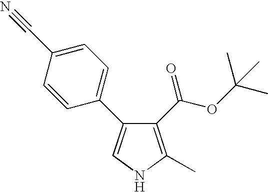

To a solution of 4-[(E)-2-nitrovinyl]benzonitrile (11.47 g) synthesized by a known method and tert-butyl acetoacetate (11.47 ml) in methanol (18 ml) was added sodium methoxide (918 mg) and the mixture was stirred at room temperature for 20 minutes. To this reaction mixture was added a 2M ammonia-methanol solution (180 ml), and the mixture was stirred at room temperature for 20 hours. The reaction mixture was concentrated, the residue was distributed between ethyl acetate and an aqueous 10% citric acid solution. The organic layer was washed with an aqueous 10% citric acid solution and a sodium chloride solution, dried over magnesium sulfate and concentrated. The residue was washed with diisopropyl ether to obtain the desired product (4.40 g).

¹H-NMR (CDCl₃) δ: 1.40 (9H, s), 2.53 (3H, s), 6.60-6.61 (1H, m), 7.45-7.48 (2H, m), 7.58-7.61 (2H, m), 8.33 (1H, s).

### REFERENCE EXAMPLE 35

### 4-(4-Cyanophenyl)-2-methyl-1H-pyrrole

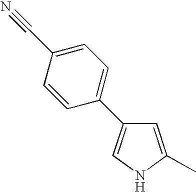

TFA (1.1 ml) was added to tert-butyl 4-(4-cyanophenyl)-2-methyl-1H-pyrrole-3-carboxylate (327 mg), and the mixture was stirred at room temperature for 1 hour. The reaction mixture was concentrated under reduced pressure, the residue was poured into a mixture of THF-ethyl acetate-aqueous saturated sodium bicarbonate solution to separate into layers. The organic layer was washed with an aqueous saturated sodium bicarbonate solution and a saturated sodium chloride solution, dried over magnesium sulfate, and concetrated. The residue was washed with diisopropyl ether to obtain the desired product (172 mg) as a solid.

¹H-NMR (DMSO-d₆) δ: 2.19 (3H, s), 6.22 (1H, s), 7.27 (1H, t), 7.66 (4H, dd), 10.93 (1H, s)

### REFERENCE EXAMPLE 36

### 3-(4-Cyanophenyl)-5-methyl-2-(trifluoromethyl)-1H-pyrrole

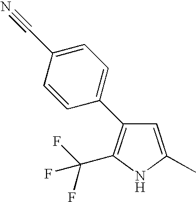

A solution of 4-(4-cyanophenyl)-2-methyl-1H-pyrrole (1.01 g), N-iodosuccinimide (1.31 g) in DMF (25 ml) was stirred at room temperature for 1 hour. The reaction mixture was diluted with ethyl acetate, washed with water and a saturated sodium chloride solution, dried over magnesium sulfate, and concentrated. The residue was purified by silica gel column chromatography (ethyl acetate-hexane) to obtain a mixture of 3-(4-cyanophenyl)-5-methyl-2-iodo-1H-pyrrole (1.17 g) and by-products. To a solution of the mixture (700 mg) in DMF (12 ml) were added copper iodide (476 mg) and methyl fluorosulfonyl(difluoro)acetate (1.92 ml), and the mixture was heated at 100° C. for 1 hour. The reaction mixture was cooled to room temperature and poured to water and the mixture was extracted with ethyl acetate (50 ml). The organic layer was washed with water and a saturated sodium chloride solution, dried over magnesium sulfate, and concentrated. The residue was purified by silica gel column chromatography (ethyl acetate-hexane) and crystallized from diisopropyl ether-hexane to obtain the desired product (92 mg) as crystals.

¹H-NMR (CDCl₃) δ: 2.34 (3H, s), 6.09-6.10 (1H, m), 7.53 (2H, d), 7.65 (2H, d), 8.40 (1H, s)

### REFERENCE EXAMPLE 37

### 3-(4-Cyanophenyl)-2-formyl-5-methyl-1H-pyrrole

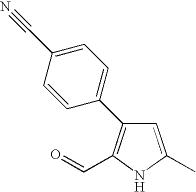

To a solution of 4-(4-cyanophenyl)-2-methyl-1H-pyrrole (200 mg) in DMF (3.0 ml) was added Vilsmeier reagent (337 mg) by portions with ice-cooling, and the mixture was stirred for 4 hours. The reaction mixture was poured into an aqueous saturated sodium bicarbonate solution (20 ml), and the mixture was extracted with ethyl acetate. The organic layer was washed with water and a saturated sodium chloride solution, dried over magnesium sulfate, and concentrated under reduced pressure. The residue was purified by silica gel chromatography (ethyl acetate-hexane) and crystallized from ethyl acetate-diisopropyl ether to obtain the desired product (148 mg) as crystals.

¹H-NMR (CDCl₃) δ: 2.42 (3H, s), 6.21 (1H, d), 7.58 (2H, d), 7.72 (2H, d), 9.50 (1H, s), 10.07 (1H, s).

### REFERENCE EXAMPLE 38

### Tert-butyl 4-(4-cyanophenyl)-2-iodo-5-methyl-1H-pyrrole-3-carboxylate

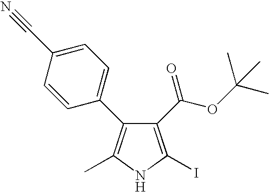

A solution of tert-butyl 4-(4-cyanophenyl)-5-methyl-1H-pyrrole-3-carboxylate (4.79 g) and N-iodosuccinimide (4.21 g) in DMF (50 ml) was stirred at room temperature for 2 hours. The reaction mixture was poured into a saturated sodium chloride solution (200 ml), and the mixture was extracted with ethyl acetate. The ethyl acetate layer was washed with a saturated saline, dried over magnesium sulfate, and concentrated. The residue was purified by silica gel column chromatography (ethyl acetate-hexane), and crystallized from ethyl acetate to obtain the desired product (5.83 g) as crystals.

¹H-NMR (CDCl₃) δ1.31 (9H, m), 2.15 (3H, s), 7.30-7.35 (2H, m), 7.61-7.67 (2H, m), 8.49 (1H, s).

### REFERENCE EXAMPLE 39

### Tert-butyl 4-(4-cyanophenyl)-5-methyl-2-(trifluoromethyl)-1H-pyrrole-3-carboxylate

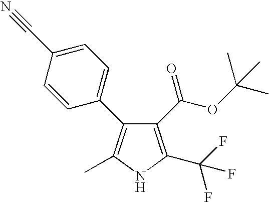

A solution of tert-butyl 4-(4-cyanophenyl)-2-iodo-5-methyl-1H-pyrrole-3-carboxylate (1.06 g), methyl fluorsulfonyldifluoroacetate (0.99 ml) and copper iodide (0.55 g) in DMF (15 ml) was stirred at 80° C. for 14 hours. The reaction mixture was poured into a saturated sodium chloride solution (200 ml), and the mixture was extracted with ethyl acetate. The ethyl acetate layer was washed with a saturated sodium chloride solution, dried over magnesium sulfate, and concentrated. The residue was purified by silica gel column chromatography (ethyl acetate-hexane) and crystallized from ethyl acetate to obtain the desired product (0.65 g) as crystals.

¹H-NMR (CDCl₃) δ 1.39 (9H, s), 2.18 (3H, s), 7.37 (2H, d), 7.67 (2H, d), 8.77 (1H, br s).

### REFERENCE EXAMPLE 40

### Ethyl 2-chloro-5-{[2-acetyl-3-(4-cyanophenyl)-5-methyl-1H-pyrrol-1-yl]methyl}nicotinate

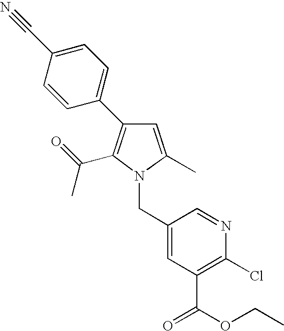

To a solution of ethyl 2-chloro-5-{[4-(4-cyanophenyl)-2-methyl-1H-pyrrol-1-yl]methyl}nicotinate (463 mg) obtained according to the same manner as that of Reference Example 24, acetyl chloride (0.14 ml) and dichloromethane (13 ml) was added dropwise tin tetrachloride (0.46 ml) with ice-cooling, and the mixture was stirred for 2 hours. The reaction mixture was poured into a cold aqueous saturated sodium bicarbonate solution, and the mixture was extracted with ethyl acetate. The organic layer was washed with an aqueous saturated sodium bicarbonate solution and a saturated sodium chloride solution, dried over magnesium sulfate and concentrated under reduced pressure. The residue was subjected to silica gel column chromatography (ethyl acetate-hexane), and crystallized from ethanol-diisopropyl ether to obtain the desired product (213 mg).

¹H NMR (CDCl₃) δ: 1.41 (3H, t), 1.91 (3H, s), 2.27 (3H, s), 4.41 (2H, q), 5.60 (2H, s), 6.05 (1H, s), 7.45 (2H, d), 7.70 (2H, d), 7.86 (1H, d), 8.10 (1H, d).

### EXAMPLE 1

### 1-{[6-Chloro-5-(hydroxymethyl)pyridin-3-yl]methyl}-4-(4-cyanophenyl)-2,5-dimethyl-1H-pyrrole-3-carbonitrile (Compound 1)

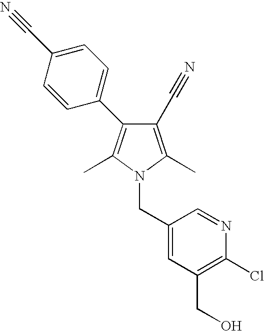

A mixture of sodium borohydride (0.50 g), calcium chloride (1.0 g), THF (40 ml) and ethanol (20 ml) was stirred at room temperature for 30 minutes, ethyl 2-chloro-5-{[3-cyano-4-(4-cyanophenyl)-2,5-dimethyl-1H-pyrrol-1-yl]methyl}nicotinate (1.8 g) was added, and this was stirred at room temperature for 14 hours. The reaction mixture was poured into an aqueous saturated citric acid solution (100 ml), this was extracted with ethyl acetate, and the ethyl acetate layer was washed with an aqueous saturated sodium chloride solution, dried over magnesium sulfate, and concentrated. The residue was purified by silica gel column chromatography (ethyl acetate-hexane), and crystallized from ethanol to obtain the desired product (1.26 g) as crystals. m.p. 147-148° C.

¹H-NMR (200 MHz, CDCl₃) δ2.22 (3H, s), 2.38 (3H, s), 2.84 (1H, br s), 4.76 (2H, s), 5.14 (2H, s), 7.50 (2H, d), 7.54 (1H, d), 7.69 (2H, d), 7.94 (1H, d).

### EXAMPLE 2

### 1-{[6-Chloro-5-(hydroxymethyl)pyridin-3-yl]methyl}-4-(4-cyanophenyl)-2-ethyl-5-methyl-1H-pyrrole-3-carbonitrile (Compound 2)

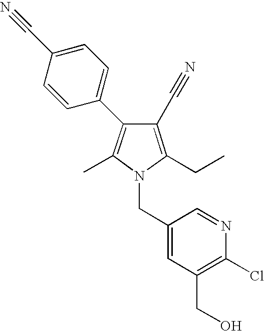

A mixture of sodium borohydride (0.14 g), calcium chloride (0.28 g), THF (10 ml) and ethanol (5 ml) was stirred at room temperature for 30 minutes, ethyl 2-chloro-5-{[3-cyano-4-(4-cyanophenyl)-2-ethyl-5-methyl-1H-pyrrol-1-yl]methyl}nicotinate (0.40 g) was added thereto, and the mixture was stirred at room temperature for 14 hours. The reaction mixture was poured into an aqueous saturated citric acid solution (50 ml), and the mixture was extracted with ethyl acetate. The ethyl acetate layer was washed with a saturated sodium chloride solution, dried over magnesium sulfate, and concentrated. The residue was purified by silica gel column chromatography (ethyl acetate-hexane) and crystallized from ethyl acetate to obtain the desired product (224 mg) as crystals. m.p. 126-127° C.

¹H NMR (CDCl₃) δ1.24 (3H, t), 2.18 (3H, s), 2.79 (2H, q), 4.78 (2H, s), 5.16 (2H, s), 7.4-7.6 (3H, m), 7.72 (2H, d), 7.92 (1H, d).

### EXAMPLE 3

### 1-{[6-Chloro-5-(hydroxymethyl)pyridin-3-yl]methyl}-4-(4-cyanophenyl)-5-methyl-2-propyl-1H-pyrrole-3-carbonitrile (Compound 3)

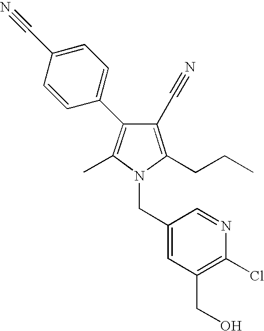

A mixture of sodium borohydride (134 mg), calcium chloride (294 mg), THF (10 ml) and ethanol (5 ml) was stirred at room temperature for 40 minutes, ethyl 2-chloro-5-{[3-cyano-4-(4-cyanophenyl)-5-methyl-2-propyl-1H-pyrrol-1-yl]methyl}nicotinate (395 mg) was added thereto, and the mixture was stirred at room temperature for 2 hours. The reaction mixture was concentrated, and partitioned between ethyl acetate and water. The organic layer was washed with an ammonium chloride solution and a saturated sodium chloride solution, dried over sodium sulfate, and concentrated. The residue was purified by silica gel column chromatography (ethyl acetate-hexane), and crystallized from ethyl acetate to obtain the desired product (325 mg) as crystals. m.p. 137-138° C.

¹H-NMR (CDCl₃) δ: 0.99 (3H, t), 1.56-1.68 (2H, m), 2.17 (3H, s), 2.20 (1H, br.s), 2.74 (2H, t), 4.78 (2H, s), 5.14 (2H, s), 7.50-7.53 (3H, m), 7.69-7.73 (2H, m), 7.91 (1H, d).

### EXAMPLE 4

### 1-{[6-Chloro-5-(hydroxymethyl)pyridin-3-yl]methyl}-4-(4-cyanophenyl)-2-isopropyl-5-methyl-1H-pyrrole-3-carbonitrile (Compound 4)

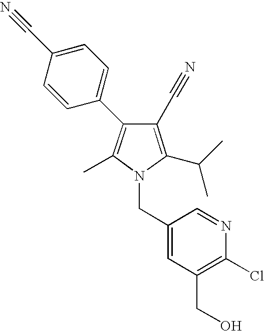

A mixture of sodium borohydride (0.33 g), calcium chloride (0.66 g), THF (25 ml) and ethanol (12 ml) was stirred at room temperature for 30 minutes, ethyl 2-chloro-5-{[3-cyano-4-(4-cyanophenyl)-2-isopropyl-5-methyl-1H-pyrrol-1-yl]methyl}nicotinate (0.95 g) was added thereto, and the mixture was stirred at room temperature for 14 hours. The reaction mixture was poured into an aqueous saturated citric acid solution (50 ml), and the mixture was extracted with ethyl acetate. The ethyl acetate layer was washed with a saturated sodium chloride solution, dried over magnesium sulfate, and concentrated. The residue was purified by silica gel column chromatography (ethyl acetate-hexane), and crystallized from ethyl acetate to obtain the desired product (375 mg) as crystals.

¹H NMR (CDCl₃) δ1.41 (6H, d), 2.18 (3H, s), 3.02 (1H, sept), 4.76 (2H, m), 5.16 (2H, d), 7.4-7.6 (3H, m), 7.72 (2H, d), 7.91 (1H, d).

### EXAMPLE 5

### 1-{[6-Chloro-5-(hydroxymethyl)pyridin-3-yl]methyl}-4-(4-cyanophenyl)-2-cyclopropyl-5-methyl-1H-pyrrole-3-carbonitrile (Compound 5)

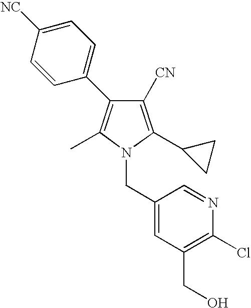

A mixture of sodium borohydride (31 mg), calcium chloride (90 mg), THF (3.0 ml) and ethanol (1.5 ml) was stirred at room temperature for 30 minutes, ethyl 2-chloro-5-{[3-cyano-4-(4-cyanophenyl)-2-cyclopropyl-5-methyl-1H-pyrrol-1-yl]methyl}nicotinate (120 mg) was added thereto, and the mixture was stirred at room temperature for 14 hours. The reaction mixture was poured into water, and the mixture was extracted with ethyl acetate. The ethyl acetate layer was washed with a saturated sodium chloride solution, dried over magnesium sulfate, and concentrated. The residue was purified by silica gel column chromatography (ethyl acetate-hexane), and crystallized from diisopropyl ether to obtain the desired product (80.0 mg) as crystals. m.p. 193.4-194.5° C.

¹H-NMR (CDCl₃) δ: 0.97-1.04 (4H, m), 1.60-1.66 (1H, m), 2.18 (4H, br), 4.78 (2H, d), 5.32 (2H, s), 7.47 (2H, d), 7.54 (1H, dd), 7.69 (2H, d), 7.96 (1H, d).

### EXAMPLE 6

### 1-{[6-Chloro-5-(hydroxymethyl)pyridin-3-yl]methyl}-4-[4-cyano-3-(trifluoromethyl)phenyl]-2,5-dimethyl-1H-pyrrole-3-carbonitrile (Compound 6)

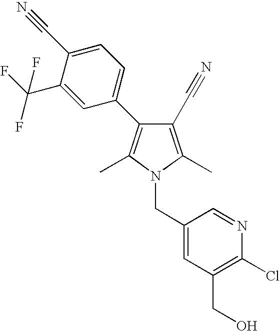

A mixture of sodium borohydride (0.20 g), calcium chloride (0.10 g), THF (25 ml) and ethanol (12 ml) was stirred at room temperature for 30 minutes, isopropyl 2-chloro-5-({3-cyano-4-[4-cyano-3-(trifluoromethyl)phenyl]-2,5-dimethyl-1H-pyrrol-1-yl}methyl)nicotinate (0.23 g) was added thereto, and the mixture was stirred at room temperature for 14 hours. The reaction mixture was poured into an aqueous saturated citric acid solution (50 ml), and the mixture was extracted with ethyl acetate. The ethyl acetate layer was washed with a saturated sodium chloride solution, dried over magnesium sulfate, and concentrated. The residue was purified by silica gel column chromatography (ethyl acetate-hexane), and crystallized from ethyl acetate to obtain the desired product (174 mg) as crystals.

¹H NMR (CDCl₃) δ 2.17 (1H, br s), 2.24 (3H, s), 2.42 (3H, s), 4.79 (2H, s), 5.15 (2H, s), 7.53 (1H, s), 7.6-7.8 (2H, m), 7.8-8.0 (2H, m).

### EXAMPLE 7

### 1-{[6-Chloro-5-(hydroxymethyl)pyridin-3-yl]methyl}-4-(3-cyanophenyl)-2,5-dimethyl-1H-pyrrole-3-carbonitrile (Compound 7)

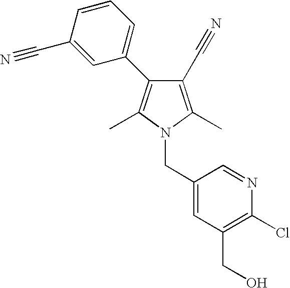

A mixture of sodium borohydride (0.12 g), calcium chloride (0.24 g), THF (8 ml) and ethanol (4 ml) was stirred at room temperature for 30 minutes, ethyl 2-chloro-5-{[3-cyano-4-(3-cyanophenyl)-2,5-dimethyl-1H-pyrrol-1-yl]methyl}nicotinate (0.31 g) was added thereto, and the mixture was stirred at room temperature for 14 hours. The reaction mixture was poured into an aqueous saturated citric acid solution, and the mixture was extracted with ethyl acetate. The ethyl acetate layer was washed with a saturated sodium chloride solution, dried over magnesium sulfate, and concentrated. The residue was purified by silica gel column chromatography (ethyl acetate-hexane), and crystallized from ethyl acetate to obtain the desired product (159 mg) as an amorphous solid.

¹H NMR (CDCl₃) δ 1.98 (1H, br s), 2.20 (3H, s), 2.40 (3H, s), 4.79 (2H, d), 5.14 (2H, s), 7.51 (1H, d), 7.63 (4H, m), 7.97 (1H, d).

### EXAMPLE 8

### 1-{[6-Chloro-5-(hydroxymethyl)pyridin-3-yl]methyl}-4-(4-cyanophenyl)-5-ethyl-2-methyl-1H-pyrrole-3-carbonitrile (Compound 8)

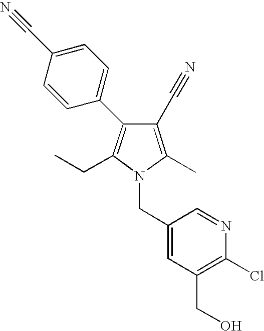

According to the same manner as that shown in Example 1 and using ethyl 2-chloro-5-{[3-cyano-4-(4-cyanophenyl)-5-ethyl-2-methyl-1H-pyrrol-1-yl]methyl}nicotinate as a starting material, the desired product was obtained.

¹H-NMR (CDCl₃) δ: 1.13 (3H, t), 2.24 (1H, s), 2.33 (3H, s), 2.58 (2H, q), 4.77 (2H, d), 5.15 (2H, s), 7.48-7.52 (3H, m), 7.70 (2H, d), 7.88 (1H, d).

### EXAMPLE 9

### Methyl 1-{[6-chloro-5-(hydroxymethyl)pyridin-3-yl]methyl}-4-(4-cyanophenyl)-5-methyl-2-(trifluoromethyl)-1H-pyrrole-3-carboxylate (Compound 9)

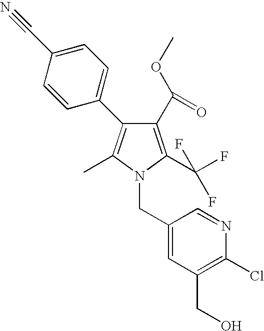

According to the same manner as that shown in Example 1 and using ethyl 2-chloro-5-{[3-(4-cyanophenyl)-4-(methoxycarbonyl)-2-methyl-5-(trifluoromethyl)-1H-pyrrol-1-yl]methyl}nicotinate as a starting material, the desired product was obtained.

¹H-NMR (CDCl₃) δ: 2.11 (3H, s), 2.18 (1H, t), 3.71 (3H, s), 4.78 (2H, d), 5.30 (2H, s), 7.36 (2H, d), 7.58 (1H, d), 7.68 (2H, d), 7.96 (1H, d)

### EXAMPLE 10

### 1-{[5-Chloro-3-(hydroxymethyl)pyridin-2-yl]methyl}-4-(4-cyanophenyl)-2,5-dimethyl-1H-pyrrole-3-carbonitrile (Compound 10)

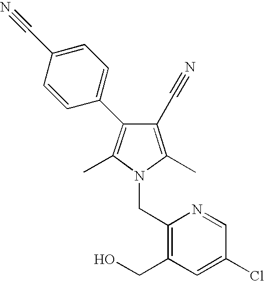

According to the same manner as that shown in Example 1 and using methyl 5-chloro-2-{[3-cyano-4-(4-cyanophenyl)-2,5-dimethyl-1H-pyrrol-1-yl]methyl}nicotinate as a starting material, the desired product was obtained.

¹H NMR (CDCl₃) δ2.16 (4H, m), 2.34 (3H, s), 4.80 (2H, d), 5.22 (2H, s), 7.53 (2H, d), 7.6-7.8 (3H, m), 8.41 (1H, d).

### EXAMPLE 11

### 1-{[5-Chloro-4-(hydroxymethyl)pyridin-2-yl]methyl}-4-(4-cyanophenyl)-2,5-dimethyl-1H-pyrrole-3-carbonitrile (Compound 11)

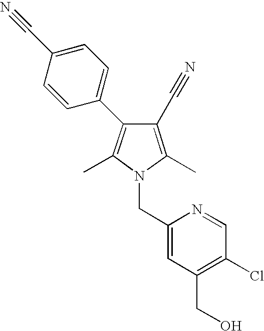

According to the same manner as that shown in Example 1 and using methyl 5-chloro-2-{[3-cyano-4-(4-cyanophenyl)-2,5-dimethyl-1H-pyrrol-1-yl]methyl}isonicotinate as a starting material, the desired product was obtained.

¹H NMR (DMSO-d₆) δ2.15 (3H, s), 2.30 (3H, m), 4.71 (2H, d,) 5.29 (2H, s), 5.71 (1H, t), 7.19 (1H, s), 7.57 (1H, s), 7.62 (2H, d), 7.93 (2H, d).

### EXAMPLE 12

### 1-{[5-Chloro-6-(hydroxymethyl)pyridin-2-yl]methyl}-4-(4-cyanophenyl)-2,5-dimethyl-1H-pyrrole-3-carbonitrile (Compound 12)

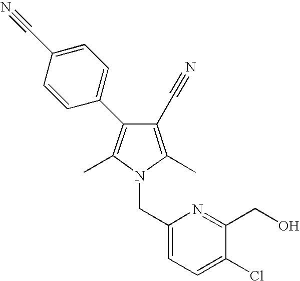

According to the same manner as that shown in Example 1 and using methyl 3-chloro-6-{[3-cyano-4-(4-cyanophenyl)-2,5-dimethyl-1H-pyrrol-1-yl]methyl}pyridine-2-carboxylate as a starting material, the desired product was obtained.

¹H NMR (CDCl₃) δ2.23 (3H, s), 2.42 (3H, s), 3.94 (1H, t), 4.81 (2H, d), 5.22 (2H, s), 6.66 (1H, d), 7.53 (2H, d), 7.6-7.8 (3H, m).

### EXAMPLE 13

### 1-{[6-Chloro-5-(hydroxymethyl)pyridin-3-yl]methyl}-4-(4-cyanophenyl)-5-methyl-1H-pyrrole-3-carbonitrile (Compound 13)

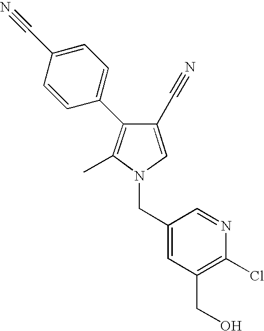

According to the same manner as that shown in Example 1 and using ethyl 2-chloro-5-{[4-cyano-3-(4-cyanophenyl)-2-methyl-1H-pyrrol-1-yl]methyl}nicotinate as a starting material, the desired product was obtained.

¹H NMR (CDCl₃) δ 2.25 (3H, s), 4.80 (2H, d), 5.13 (2H, s), 7.26 (1H, s), 7.50 (2H, d), 7.67 (1H, d), 7.72 (2H, d), 8.12 (1H, d).

### EXAMPLE 14

### 1-{[6-Chloro-5-(hydroxymethyl)pyridin-3-yl]methyl}-4-(4-cyanophenyl)-5-methyl-2-(1,3-oxazol-5-yl)-1H-pyrrole-3-carbonitrile (Compound 14)

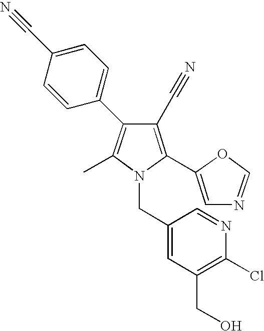

According to the same manner as that shown in Example 1 and using ethyl 2-chloro-5-{[3-cyano-4-(4-cyanophenyl)-5-methyl-2-(1,3-oxazol-5-yl)-1H-pyrrol-1-yl]methyl}nicotinate as a starting material, the desired product was obtained.

¹H NMR (CDCl₃) δ 2.15 (1H, br s), 2.28 (3H, s), 4.78 (2H, d), 5.36 (2H, s), 7.5-7.6 (4H, m), 7.77 (2H, d), 7.97 (1H, s), 8.02 (1H, d).

### EXAMPLE 15

### 1-{[6-Chloro-5-(hydroxymethyl)pyridin-3-yl]methyl}-4-(4-cyanophenyl)-2-[(dimethylamino)methyl]-5-methyl-1H-pyrrole-3-carbonitrile (Compound 15)

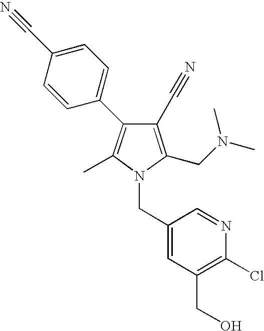

According to the same manner as that shown in Example 1 and using ethyl 2-chloro-5-({3-cyano-4-(4-cyanophenyl)-2-[(dimethylamino)methyl]-5-methyl-1H-pyrrol-1-yl}methyl)nicotinate as a starting material, the desired product was obtained.

¹H NMR (CDC₃) δ 2.18 (3H, s), 2.2-2.4 (7H, m), 3.49 (2H, s), 4.78 (2H, s), 5.39 (2H, s), 7.51 (2H, d), 7.60 (1H, d), 7.72 (2H, d), 8.00 (1H, d).

### EXAMPLE 16

### 1-{[6-Chloro-5-(hydroxymethyl)pyridin-3-yl]methyl}-4-(4-cyanophenyl)-5-methyl-2-(methylthio)-1H-pyrrole-3-carbonitrile (Compound 16)

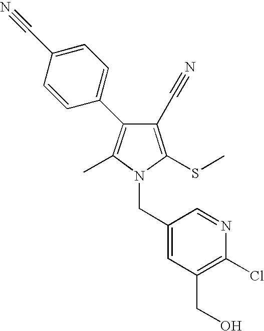

According to the same manner as that shown in Example 1 and using ethyl 2-chloro-5-{[3-cyano-4-(4-cyanophenyl)-5-methyl-2-(methylthio)-1H-pyrrol-1-yl]methyl}nicotinate as a starting material, the desired product was obtained.

¹H NMR (CDCl₃) δ 2.23 (3H, s), 2.41 (3H, s), 4.79 (2H, d), 5.40 (2H, s), 7.51 (2H, d), 7.60 (1H, d), 7.73 (2H, d), 8.02 (1H, d).

### EXAMPLE 17

### 1-{[6-Chloro-5-(hydroxymethyl)pyridin-3-yl]methyl}-4-(4-cyanophenyl)-5-methyl-2-(methylsulfonyl)-1H-pyrrole-3-carbonitrile (Compound 17)

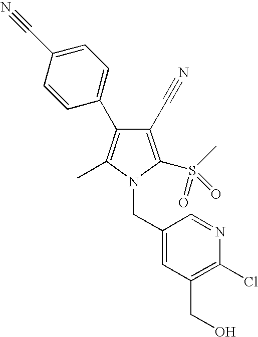

According to the same manner as that shown in Example 1 and using ethyl 2-chloro-5-{[3-cyano-4-(4-cyanophenyl)-5-methyl-2-(methylsulfonyl)-1H-pyrrol-1-yl]methyl}nicotinate as a starting material, the desired product was obtained.

¹H NMR (CDCl₃) δ 2.14 (1H, br s), 2.28 (3H, s), 3.16 (3H, s), 4.79 (2H, s), 5.67 (2H, s), 7.51 (2H, d), 7.68 (1H, d), 7.78 (2H, d), 8.03 (1H, d)

### EXAMPLE 18

### 1-{[6-Chloro-5-(hydroxymethyl)pyridin-3-yl]methyl}-4-(4-cyanophenyl)-5-methyl-2-(trifluoromethyl)-1H-pyrrole-3-carbonitrile (Compound 18)

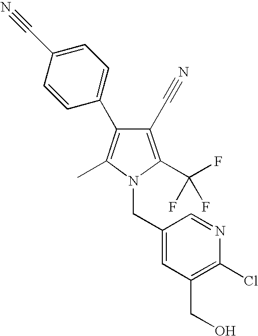

According to the same manner as that shown in Example 1 and using ethyl 2-chloro-5-{[3-cyano-4-(4-cyanophenyl)-5-methyl-2-(trifluoromethyl)-1H-pyrrol-1-yl]methyl}nicotinate as a starting material, the desired product was obtained.

¹H-NMR (CDCl₃) δ: 2.19 (4H, br s), 4.78 (2H, s), 5.32 (2H, s), 7.48-7.51 (2H, m), 7.56-7.57 (1H, m), 7.73-7.76 (2H, m), 7.95 (1H, d).

### EXAMPLE 19

### 1-{[6-Chloro-5-(hydroxymethyl)pyridin-3-yl]methyl}-4-(4-cyanophenyl)-5-methyl-2-(2,2,2-trifluoro-1-hydroxyethyl)-1H-pyrrole-3-carbonitrile (Compound 19)

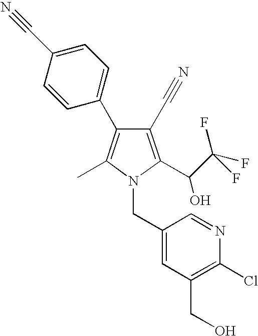

According to the same manner as that shown in Example 1 and using ethyl 2-chloro-5-{[3-cyano-4-(4-cyanophenyl)-5-methyl-2-(2,2,2-trifluoro-1-hydroxyethyl)-1H-pyrrol-1-yl]methyl}nicotinate as a starting material, the desired product was obtained.

¹H NMR (DMSO-D₆) δ 2.10 (3H, s), 4.52 (2H, d), 5.5-5.7 (3H, m), 7.54 (1H, d), 7.6-7.7 (3H, m), 7.9-8.0 (3H, m).

### EXAMPLE 20

### 1-{[6-Chloro-5-(hydroxymethyl)pyridin-3-yl]methyl}-4-(4-cyanophenyl)-2-(1,3-dioxolan-2-yl)-5-methyl-1H-pyrrole-3-carbonitrile (Compound 20)

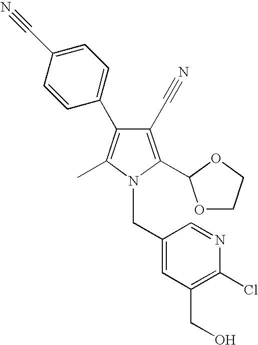

According to the same manner as that shown in Example 1 and using ethyl 2-chloro-5-{[3-cyano-4-(4-cyanophenyl)-2-(1,3-dioxolan-2-yl)-5-methyl-1H-pyrrol-1-yl]methyl}nicotinate as a starting material, the desired product was obtained.

¹H NMR (CDCl₃) δ 2.14 (3H, s), 2.23 (1H, t), 4.0-4.2 (4H, m), 4.77 (2H, d), 5.31 (2H, s), 6.03 (1H, s), 7.49 (2H, d), 7.65 (1H, d), 7.72 (2H, d), 8.08 (1H, d)

### EXAMPLE 21

### 1-{[6-Chloro-5-(hydroxymethyl)pyridin-3-yl]methyl}-4-(4-cyanophenyl)-2-(methoxymethyl)-5-methyl-1H-pyrrole-3-carbonitrile (Compound 21)

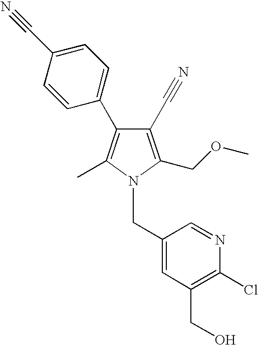

According to the same manner as that shown in Example 1 and using ethyl 2-chloro-5-{[3-cyano-4-(4-cyanophenyl)-2-(methoxymethyl)-5-methyl-1H-pyrrol-1-yl]methyl}nicotinate as a starting material, the desired product was obtained.

¹H NMR (CDCl₃) δ 2.10 (1H, br s), 2.19 (3H, s), 3.38 (3H, s), 4.53 (2H, s), 4.78 (2H, d), 5.26 (2H, s), 7.51 (2H, d), 7.61 (1H, d), 7.73 (2H, d), 8.00 (1H, d).

### EXAMPLE 22

### 1-{[6-Chloro-5-(hydroxymethyl)pyridin-3-yl]methyl}-4-(4-cyanophenyl)-2-(ethoxymethyl)-5-methyl-1H-pyrrole-3-carbonitrile (Compound 22)

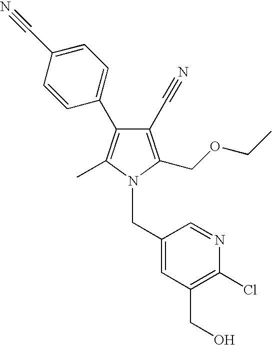

According to the same manner as that shown in Example 1 and using ethyl 2-chloro-5-{[3-cyano-4-(4-cyanophenyl)-2-(ethoxymethyl)-5-methyl-1H-pyrrol-1-yl]methyl}nicotinate as a starting material, the desired product was obtained.

¹H NMR (CDCl₃) δ 1.16 (3H, t), 2.17 (1H, br s), 2.19 (3H, s), 3.56 (2H, q), 4.58 (2H, s), 4.77 (2H, s), 5.28 (2H, s), 7.51 (2H, d), 7.62 (1H, d), 7.73 (2H, d), 8.01 (1H, d).

### EXAMPLE 23

### 1-{[6-Chloro-5-(hydroxymethyl)pyridin-3-yl]methyl}-4-(4-cyanophenyl)-5-methyl-2-[(methylthio)methyl]-1H-pyrrole-3-carbonitrile (Compound 23)

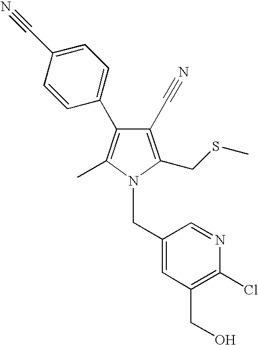

According to the same manner as that shown in Example 1 and using ethyl 2-chloro-5-({3-cyano-4-(4-cyanophenyl)-5-methyl-2-[(methylthio)methyl]-1H-pyrrol-1-yl}methyl)nicotinate as a starting material, the desired product was obtained.

¹H NMR (CDCl₃) δ 2.06 (1H, t), 2.15 (3H, s), 2.20 (3H, s), 3.76 (2H, s), 4.79 (2H, d), 5.32 (2H, s), 7.4-7.6 (3H, m), 7.73 (2H, d), 7.94 (1H, d).

### EXAMPLE 24

### 1-{[6-Chloro-5-(hdyroxymethyl)pyridin-3-yl]methyl}-4-(4-cyanophenyl)-5-methyl-2-[(methylsulfonyl)methyl]-1H-pyrrole-3-carbonitrile (Compound 24)

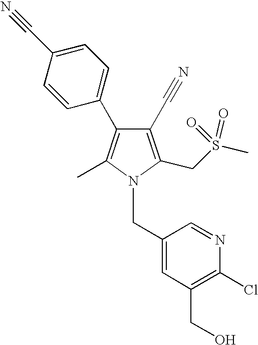

According to the same manner as that shown in Example 1 and using ethyl 2-chloro-5-({3-cyano-4-(4-cyanophenyl)-5-methyl-2-[(methylsulfonyl)methyl]-1H-pyrrol-1-yl}methyl)nicotinate as a starting material, the desired product was obtained.

¹H NMR (CDCl₃) δ 2.1-2.3 (1H, m), 2.26 (3H, s), 3.05 (3H, s), 4.39 (2H, s), 4.79 (2H, s), 5.47 (2H, s), 7.4-7.6 (3H, m), 7.76 (2H, d), 7.85 (1H, d).

### EXAMPLE 25

### 1-[4-Chloro-3-(hydroxymethyl)benzyl]-4-(3-cyanophenyl)-2,5-dimethyl-1H-pyrrole-3-carbonitrile (Compound 25)

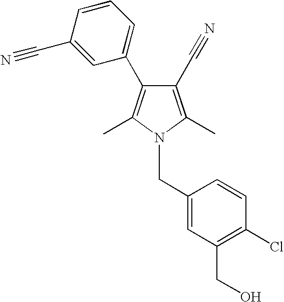

According to the same manner as that shown in Example 1 and using methyl 2-chloro-5-{[3-cyano-4-(3-cyanophenyl)-2,5-dimethyl-1H-pyrrol-1-yl]methyl}benzoate as a starting material, the desired product was obtained.

¹H NMR (CDCl₃) δ 1.98 (1H, br s), 2.17 (3H, s), 2.37 (3H, s), 4.79 (2H, s), 5.10 (2H, s), 6.71 (1H, dd), 7.21 (1H, d), 7.35 (1H, d), 7.59 (2H, d), 7.65 (1H, s), 7.69 (1H, m).

### EXAMPLE 26

### 1-[4-Chloro-3-(hydroxymethyl)benzyl]-4-(4-cyanophenyl)-2,5-dimethyl-1H-pyrrole-3-carbonitrile (Compound 26)

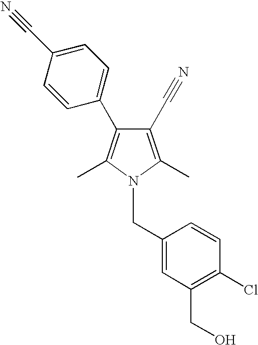

A mixture of sodium borohydride (164 mg), calcium chloride (481 mg), THF (20 ml) and ethanol (10 ml) was stirred at room temperature for 30 minutes, methyl 2-chloro-5-{[3-cyano-4-(4-cyanophenyl)-2,5-dimethyl-1H-pyrrol-1-yl]methyl}benzoate (700 mg) was added thereto, and the mixture was stirred at room temperature for 16 hours. The reaction mixture was poured into an aqueous saturated citric acid solution (100 ml), and the mixture was extracted with ethyl acetate. The ethyl acetate layer was washed with a saturated sodium chloride solution, dried over magnesium sulfate, and concentrated. The residue was purified by silica gel column chromatography (ethyl acetate-hexane), and crystallized from diethyl ether to obtain the desired product (558 mg) as crystals. m.p. 189° C.

¹H-NMR (200 MHz, CDCl₃) δ 1.98 (1H, t), 2.20 (3H, s), 2.37 (3H, s), 4.79 (2H, d), 5.10 (2H, s), 6.72 (1H, dd), 7.21 (1H, d), 7.34 (1H, d), 7.52 (2H, d), 7.72 (2H, d).

### EXAMPLE 27

### 4-(4-cyanophenyl)-2,5-dimethyl-1-[4-Fluoro-3-(hydroxymethyl)benzyl]-1H-pyrrole-3-carbonitrile (Compound 27)

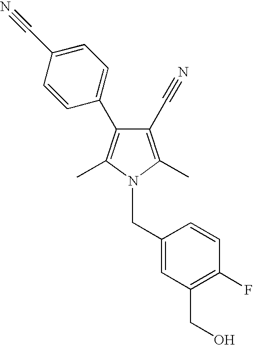

According to the same manner as that shown in Example 1 and using ethyl 5-{[3-cyano-4-(4-cyanophenyl)-2,5-dimethyl-1H-pyrrol-1-yl]methyl}-2-fluorobenzoate as a starting material, the desired product was obtained.

¹H-NMR (CDCl₃) δ: 1.85(1H, t), 2.20 (3H,S), 2.37 (3H, s), 4.76 (2H, d), 5.08 (2H, s), 6.72-6.78 (1H, m), 7.03 (1H, t), 7.11 (1H, dd), 7.51 (2H, d), 7.70 (2H, d).

### EXAMPLE 28

### 1-[4-Chloro-3-(hydroxymethyl)benzyl]-4-(4-cyanophenyl)-2-ethyl-5-methyl-1H-pyrrole-3-carbonitrile (Compound 28)

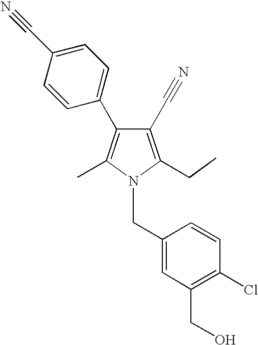

Sodium hydride (60% oil suspension, 0.16 g) was added to a solution of 4-(4-cyanophenyl)-2-ethyl-5-methyl-1H-pyrrole-3-carbonitrile (0.40 g) in DMF (10 ml) under ice-cooling. The reaction mixture was stirred at room temperature for 30 minutes, methyl 5-(bromomethyl)-2-chlorobenzoate (0.54 g) was added, and this was stirred at room temperature for 4 hours. The reaction mixture was poured into an aqueous saturated sodium chloride solution, this was extracted with ethyl acetate, and the ethyl acetate layer was dried over magnesium sulfate, and concentrated to obtain an oil (0.25 g). A mixture of sodium borohydride (0.26 g), calcium chloride (0.52 g), THF (10 ml) and ethanol (5 ml) was stirred at room temperature for 30 minutes, the oil (0.25 g) was added, and this was stirred at room temperature for 14 hours. The reaction mixture was poured into an aqueous saturated citric acid solution (10 ml), this was extracted with ethyl acetate, and the ethyl acetate layer was washed with an aqueous saturated sodium chloride solution, dried over magnesium sulfate, and concentrated. The residue was purified by silica gel column chromatography (ethyl acetate-hexane), and crystallized from ethanol to obtain the desired product (43 mg) as crystals.

¹H NMR (CDCl₃) δ 1.22 (3H, t), 2.00 (1H, br s), 2.16 (3H, s), 2.77 (2H, q), 4.79 (2H, s), 5.12 (2H, s), 6.68 (1H, dd), 7.21 (1H, d), 7.34 (1H, d), 7.53 (2H, d), 7.71 (2H, d).

### EXAMPLE 29

### 1-[4-Chloro-3-(3-hydroxypropyl)benzyl]-4-(4-cyanophenyl)-2,5-dimethyl-1H-pyrrole-3-carbonitrile (Compound 29)

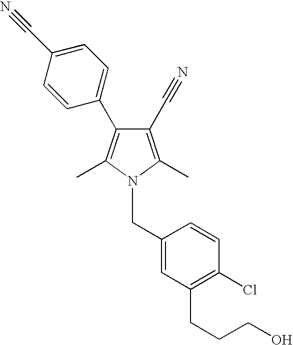

A mixture of the compound 26 (2.0 g), manganese dioxide (16.3 g) and dichloromethane (80 ml) was stirred at room temperature for 5 hours. The reaction mixture was filtered with Celite, and the filtrate was concentrated under reduced pressure to obtain 1-(4-chloro-3-formylbenzyl)-4-(4-cyanophenyl)-2,5-dimethyl-1H-pyrrole-3-carbonitrile (1.39 g).

A solution of 1-(4-chloro-3-formylbenzyl)-4-(4-cyanophenyl)-2,5-dimethyl-1H-pyrrole-3-carbonitrile (465 mg) in acetonitrile (4 ml) was added to a mixture of ethyl diethylphosphonoacetate (271 μg), 1,8-diazabicyclo[5.4.0]undec-7-ene (205 μl), and acetonitrile (6 ml), and this was stirred at room temperature for 1 hour. The reaction mixture was concentrated, and the residue was purified by silica gel column chromatography (ethyl acetate-hexane), and crystallized from diethyl ether to obtain ethyl (2E)-3-(2-chloro-5-{[3-cyano-4-(4-cyanophenyl)-2,5-dimethyl-1H-pyrrol-1-yl]methyl}phenyl)acrylate (410 mg).

A mixture of calcium chloride (525 mg), sodium borohydride (398 mg), ethanol (3 ml) and THF (6 ml) was stirred at room temperature for 10 minutes, and a solution (1 ml) of ethyl (2E)-3-(2-chloro-5-{[3-cyano-4-(4-cyanophenyl)-2,5-dimethyl-1H-pyrrol-1-yl]methyl}phenyl)acrylate (350 mg) in THF was added. The mixture was stirred at room temperature for 3 hours, the reaction mixture was concentrated, and the residue was distributed between ethyl acetate and water. The organic layer was washed with an ammonium chloride solution and an aqueous sodium chloride solution, dried (magnesium sulfate) and concentrated. The residue was purified by silica gel column chromatography (ethyl acetate-hexane) to obtain the desired product (80 mg) as an amorphous substance.

¹H-NMR (CDCl₃) δ: 1.35 (1H, br s), 1.81-1.90 (2H, m), 2.19 (3H, s), 2.37 (3H, s), 2.81 (2H, dd), 3.69 (2H, br s), 5.05 (2H, s), 6.64 (1H, dd), 6.86 (1H, d), 7.31 (1H, d), 7.52 (2H, d), 7.70 (2H, d).

### EXAMPLE 30

### (2-Chloro-5-{[3-cyano-4-(4-cyanophenyl)-2-cyclopropyl-5-methyl-1H-pyrrol-1-yl]methyl}pyridin-3-yl)methyl N,N-dimethylglycinate (Compound 30)

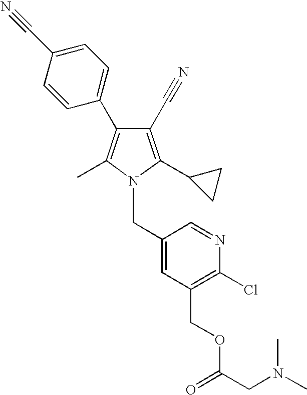

A mixture of Compound 5 (380 mg), N,N-dimethylglycine (117 mg), WSC (271 mg), DMAP (138 mg) and DMF (12 ml) was stirred at room temperature for 2 days. The reaction mixture was poured into water, and this was extracted with ethyl acetate. The extract was washed with water, dried (sodium sulfate) and concentrated. The residue was purified by silica gel column chromatography (ethyl acetate-hexane), and crystallized from diethyl ether to obtain the desired product (373 mg) as crystals.

¹H-NMR (CDCl₃) δ: 0.94-1.07 (4H, m), 1.58-1.67 (1H, m), 2.17 (3H, s), 2.33 (6H, s), 3.21 (2H, s), 5.23 (2H, s), 5.31 (2H, s), 7.30 (1H, d), 7.47-7.51 (2H, m), 7.68-7.72 (2H, m), 8.03 (1H, d).

### EXAMPLE 31

### 2-Chloro-5-{[3-cyano-4-(4-cyanophenyl)-2,5-dimethyl-1H-pyrrol-1-yl]methyl}benzyl-N,N-dimethylglycinate (Compound 31)

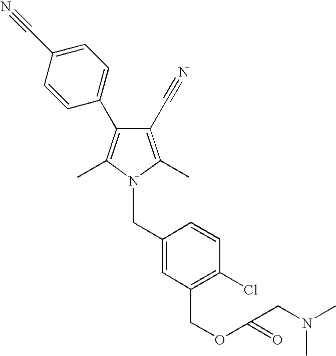

A mixture of Compound 26 (510 mg), N,N-dimethylglycine (155 mg), WSC (345 mg), pyridine (4 ml) and DMF (4 ml) was stirred at room temperature for 3 days, and at 70° C. for 5 hours. The reaction mixture was poured into a mixture of water and ethyl acetate, and the ethyl acetate layer was separated. The ethyl acetate layer was extracted with a 1N hydrochloric acid, and the hydrochloric acid layer extracted was neutralized with an aqueous saturated sodium bicarbonate solution. The aqueous layer thus neutralized was extracted with ethyl acetate, and the extract was dried over sodium sulfate and concentrated. The residue was purified by silica gel column chromatography (ethyl acetate-hexane) to obtain the desired product (220 mg) as an amorphous solid.

¹H-NMR (CDCl₃) δ: 2.19 (3H, s), 2.34 (6H, s), 2.37 (3H, s), 3.22 (2H, s), 5.09 (2H, s), 5.25 (2H, s), 6.77 (1H, dd), 7.03 (1H, d), 7.38 (1H, d), 7.53 (2H, d), 7.72 (2H, d).

### EXAMPLE 32

### 4-[(2-Chloro-5-{[3-cyano-4-(4-cyanophenyl)-2,5-dimethyl-1H-pyrrol-1-yl]methyl}benzyl)oxy]-4-oxobutanoic acid (Compound 32)

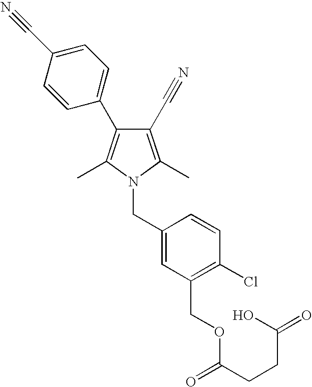

Synthesis method 1: A mixture of Compound 26 (6.10 g), 4-tert-butoxy-4-oxobutanoic acid (3.67 g), WSC (4.67 g) and pyridine (50 ml) was stirred at room temperature for 7 hours. WSC (12.3 g) was added, and the mixture was stirred at 65° C. for 16 hours. The reaction mixture was concentrated, and distributed between ethyl acetate and water. The organic layer was washed with a 10% citric acid solution and a sodium bicarbonate solution, dried (sodium sulfate), and concentrated. The residue was purified by silica gel column chromatography (ethyl acetate-hexane) to obtain 2-chloro-5-{[3-cyano-4-(4-cyanophenyl)-2,5-dimethyl-1H-pyrrol-1-yl]methyl}benzyl tert-butyl succinate (9.00 g). Trifluoroacetic acid (30 ml) was added to a mixture of this compound (9.00 g) and acetonitrile (60 ml), and the mixture was stirred at room temperature for 3 hours. The reaction mixture was concentrated, and ethyl acetate and a sodium bicarbonate solution were added. This was neutralized with a 10% citric acid solution, followed by distribution. The organic layer was dried (sodium sulfate), and concentrated. The residue was purified by silica gel column chromatography (ethyl acetate-hexane), and crystallized from ethyl acetate-diisopropyl ether to obtain the desired product (5.82 g) as crystals.

Synthesis method 2: A mixture of Compound 26 (969 mg), succinic acid anhydride (516 mg) and pyridine (16 ml) was stirred at 90° C. for 16 hours. The reaction mixture was cooled to room temperature, and poured into a 5% citric acid solution, and this was extracted with ethyl acetate. The extract was washed with a 5% citric acid solution and water, dried (sodium sulfate) and concentrated. The residue was purified by silica gel column chromatography (ethyl acetate), and crystallized from ethyl acetate to obtain the desired product (880 mg) as crystals. m.p. 149-151° C.

¹H-NMR (CDCl₃) δ: 2.19 (3H, s), 2.36 (3H, s), 2.70 (4H, s), 5.09 (2H, s), 5.22 (2H, s), 6.75 (1H, dd), 7.02 (1H, d), 7.36 (1H, d), 7.52 (2H, d), 7.70 (2H, d).

### EXAMPLE 33

### 4-[(2-Chloro-5-{[3-cyano-4-(4-cyanophenyl)-5-methyl-2-propyl-1H-pyrrol-1-yl]methyl}pyridin-3-yl)methoxy]-4-oxobutanoic acid (Compound 33)

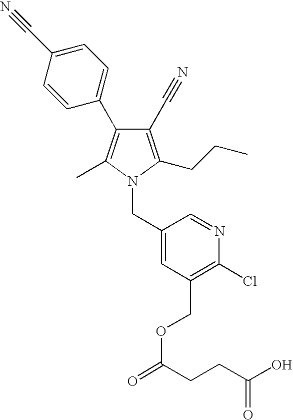

A mixture of Compound 3 (340 mg), succinic anhydride (168 mg) and pyridine (7.0 ml) was stirred at 90° C. for 11 hours. The reaction mixture was cooled to room temperature and poured into an aqueous 5% citric acid solution. The mixture was extracted with ethyl acetate, and the extract was washed with an aqueous 5% citric acid solution and water, dried over sodium sulfate and concentrated. The residue was purified by silica gel column chromatography (ethyl acetate) and crystallized from ethyl acetate to obtain the desired product (398 mg) as crystals. m.p. 167-168° C.

¹H-NMR (CDCl₃) δ: 0.99 (3H, t), 1.54-1.67 (2H, m), 2.15 (3H, s), 2.66-2.76 (6H, m), 5.14 (2H, s), 5.20 (2H, s), 7.27 (1H, d), 7.50-7.53 (2H, m), 7.69-7.72 (2H, m), 7.95 (1H, d).

### EXAMPLE 34

### 4-[(2-Chloro-5-{[3-cyano-4-(4-cyanophenyl)-2-ethyl-5-methyl-1H-pyrrol-1-yl]methyl}pyridin-3-yl)methoxy]-4-oxobutanoic acid (Compound 34)

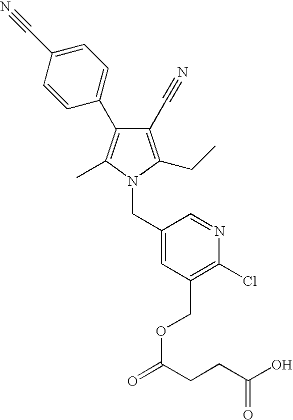

A mixture of Compound 2 (80 mg), succinic anhydride (41 mg) and pyridine (1.8 ml) was stirred at 90° C. for 13 hours. The reaction mixture was cooled to room temperature and poured into an aqueous 5% citric acid solution. The mixture was extracted with ethyl acetate, and the extract was washed with an aqueous 5% citric acid solution and water, dried over sodium sulfate and concentrated. The residue was purified by silica gel column chromatography (ethyl acetate) and crystallized from ethyl acetate to obtain the desired product (89 mg) as crystals. m.p. 187-188° C.

¹H-NMR (CDCl₃) δ: 1.23 (3H, t), 2.16 (3H, s), 2.66-2.72 (4H, m), 2.78 (2H, q), 5.15 (2H, s), 5.21 (2H, s), 7.28 (1H, d), 7.49-7.52 (2H, m), 7.69-7.72 (2H, m), 7.96 (1H, d).

### EXAMPLE 35

### 4-[(2-Chloro-5-{[3-cyano-4-(4-cyanophenyl)-2-cyclopropyl-5-methyl-1H-pyrrol-1-yl]methyl}pyridin-3-yl)methoxy]-4-oxobutanoic acid (Compound 35)

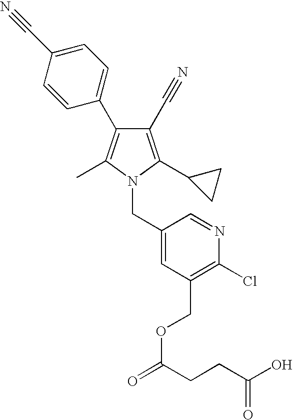

A mixture of Compound 5 (250 mg), succinic anhydride (124 mg) and pyridine (5.0 ml) was stirred at 90° C. for 13 hours. The reaction mixture was cooled to room temperature and poured into an aqueous 5% citric acid solution. The mixture was extracted with ethyl acetate, and the extract was washed with an aqueous 5% citric acid solution and water, dried over sodium sulfate and concentrated. The residue was purified by silica gel column chromatography (ethyl acetate) and crystallized from ethyl acetate to obtain the desired product (264 mg) as crystals. m.p. 188-189° C.

¹H-NMR (DMSO-d₆) δ: 0.81-0.84 (2H, m), 0.95-0.98 (2H, m), 1.80-1.90 (1H, m), 2.17 (3H, s), 2.46-2.57 (4H, m), 5.15 (2H, s), 5.47 (2H, s), 7.55-7.58 (3H, m), 7.90 (2H, d), 8.14 (1H, m), 12.2 (1H, br s).

### EXAMPLE 36

### (2-Chloro-5-{[3-cyano-4-(4-cyanophenyl)-2,5-dimethyl-1H-pyrrol-1-yl]methyl}pyridin-3-yl)methyl carbamate (Compound 36)

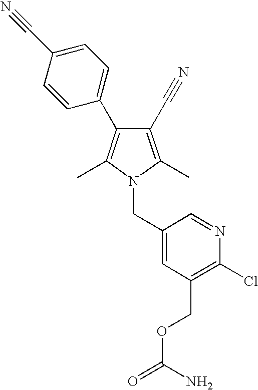

Trichloroacetyl isocyanate (0.072 ml) was added dropwise to a solution of Compound 1 (0.15 g) in dichloromethane (3 ml) under ice-cooling. This mixture was stirred for 2 hours under ice-cooling, potassium carbonate (0.11 g), methanol (2 ml) and water (2 ml) were added, and the mixture was further stirred at room temperature for 3 hours. The reaction mixture was poured into an aqueous saturated sodium chloride solution (10 ml), this was extracted with ethyl acetate, and the ethyl acetate layer was washed with an aqueous saturated sodium chloride solution, dried over magnesium sulfate, and concentrated. The residue was crystallized from ethyl acetate to obtain the desired product (0.14 g) as crystals.

¹H NMR (CDCl₃) δ2.20 (3H, s), 2.40 (3H, s), 4.72 (2H, br s), 5.13 (2H, s), 5.18 (2H, s), 7.24 (1H, s), 7.52 (2H, d), 7.73 (2H, d), 8.01 (1H, d).

### EXAMPLE 37

### 1-[4-Chloro-3-(2,2,2-trifluoro-1-hydroxyethyl)benzyl]-4-(4-cyanophenyl)-2,5-dimethyl-1H-pyrrole-3-carbonitrile (Compound 37)

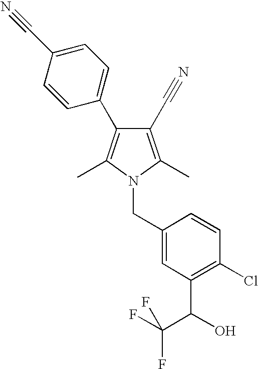

Tetrabutylammonium fluoride monohydrate (2 mg) was added to a mixture of 1-(4-chloro-3-formylbenzyl)-4-(4-cyanophenyl)-2,5-dimethyl-1H-pyrrole-3-carbonitrile (200 mg), trifluorotrimethylsilane (0.1 ml) and THF (2.5 ml) under ice-cooling, and the mixture was stirred for 4 hours. 1N hydrochloric acid (5 ml) was added to the reaction mixture, and this was extracted with ethyl acetate (20 ml). The organic layer was washed with water and an aqueous saturated sodium chloride solution, purified by basic silica gel column chromatography (ethyl acetate-hexane), and crystallized from diisopropyl ether-hexane to obtain the desired product (73 mg) as colorless crystals. m.p. 196.5-197.5° C.

¹H-NMR (DMSO-d₆) δ: 2.19 (3H, s), 2.36 (3H, s), 5.30 (2H, s), 5.44 (1H, q), 6.96 (1H, dd), 7.00 (1H, d), 7.43-7.45 (2H, m), 7.55 (2H, d), 7.83 (2H, d).

### EXAMPLE 38

### 1-{[6-Chloro-5-(2,2,2-trifluoro-1-hydroxyethyl)pyridin-3-yl]methyl}-4-(4-cyanophenyl)-2,5-dimethyl-1H-pyrrole-3-carbonitrile (Compound 38)

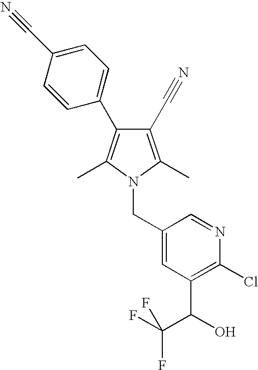

A mixture of Compound 1 (1.80 g), manganese dioxide (14.7 g) and dichloromethane (100 ml) was stirred at room temperature for 20 hours. The reaction mixture was filtered through a pad of Celite, and the filtrate was concentrated under reduced pressure to obtain 1-[(6-chloro-5-formylpyridin-3-yl)methyl]-4-(4-cyanophenyl)-2,5-dimethyl-1H-pyrrole-3-carbonitrile (554 mg). To a mixture of this compound (400 mg), (trifluoromethyl)trimethylsilane (0.19 ml) and THF (5.0 ml) was added a 1.0M solution of tetrabutylammonium bromide in THF (0.1 ml) with ice-cooling, and the mixture was stirred for 4 hours. To the reaction mixture was added a 1N hydrochloric acid (10 ml), and the mixture was extracted with ethyl acetate (30 ml). The organic layer was washed with water and a saturated sodium chloride solution, and purified by basic silica gel column chromatography (ethyl acetate-hexane) and crystallized from ethyl acetate-diisopropyl ether to obtain the desired product (184 mg) as colorless crystals. m.p. 200-201° C.

¹H-NMR (DMSO-d₆) δ 2.18 (3H, s), 2.36 (3H, s), 5.44 (3H, br), 7.35 (1H, br d), 7.57 (2H, d), 7.73 (1H, br d), 7.92 (2H, d), 8.18 (1H, d)

### EXAMPLE 39

### 1-{[6-Chloro-5-(1-hydroxyethyl)pyridin-3-yl]methyl}-4-(4-cyanophenyl)-2,5-dimethyl-1H-pyrrole-3-carbonitrile (Compound 39)

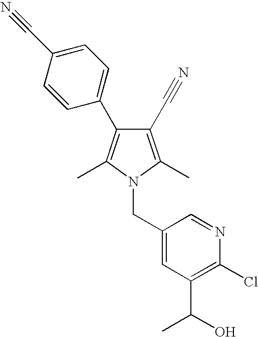

A 3N solution (0.12 ml) of methylmagnesium bromide in THF was added dropwise to a solution of 1-[(6-chloro-5-formylpyridin-3-yl)methyl]-4-(4-cyanophenyl)-2,5-dimethyl-1H-pyrrole-3-carbonitrile (0.11 g) in THF (5 ml) under ice-cooling, and this was stirred at room temperature for 20 minutes. The reaction mixture was poured into an aqueous saturated sodium chloride solution (10 ml), this was extracted with ethyl acetate, and the ethyl acetate layer was washed with an aqueous saturated sodium chloride solution, dried over magnesium sulfate, and concentrated. The residue was purified by silica gel column chromatography (ethyl acetate-hexane) to obtain the desired product (0.12 g) as an oil.

¹H NMR (CDCl₃) δ1.50 (3H, d), 2.22 (3H, s), 2.40 (3H, s), 5.13 (2H, s), 5.1-5.3 (2H, m), 7.51 (2H, d), 7.64 (1H, d), 7.71 (2H, d), 7.87 (1H, d).

### EXAMPLE 40

### 1-{[6-Chloro-5-(hydroxymethyl)pyridin-3-yl]methyl}-4-(4-cyanophenyl)-2-formyl-5-methyl-1H-pyrrole-3-carbonitrile (Compound 40)

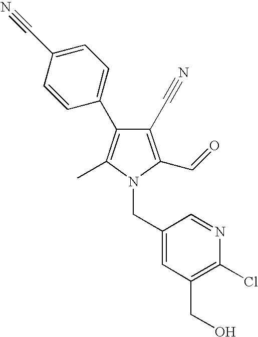

A solution of Compound 20 (0.19 g) in acetic acid (0.2 ml) and water (0.8 ml) was stirred at 50° C. for 1 hour. The reaction mixture was poured into an aqueous saturated sodium bicarbonate solution (5 ml), this was extracted with ethyl acetate, and the ethyl acetate layer was washed with an aqueous saturated sodium chloride solution, dried over magnesium sulfate, and concentrated. The residue was crystallized from ethanol to obtain the desired product (0.15 g) as crystals.

¹H NMR (200 MHz, CDCl₃) δ 2.18 (1H, br s), 2.33 (3H, s), 4.77 (2H, d), 5.74 (2H, s), 7.53 (2H, d), 7.66 (1H, d), 7.79 (2H, d), 8.06 (1H, d), 9.89 (1H, s).

### EXAMPLE 41

### 1-{[6-Chloro-5-(hydroxymethyl)pyridin-3-yl]methyl}-4-(4-cyanophenyl)-2-(1-hydroxyethyl)-5-methyl-1H-pyrrole-3-carbonitrile (Compound 41)

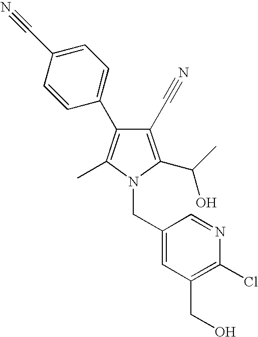

A 3N solution (0.18 ml) of methymagensium bromide in THF was added to a solution of Compound 40 (68 mg) in THF (3 ml) under ice-cooling and the mixture was stirred for 20 minutes under ice-cooling. The reaction mixture was poured into an aqueous saturated sodium chloride solution (10 ml), this was extracted with ethyl acetate, and the ethyl acetate layer was washed with an aqueous saturate sodium chloride solution, dried over magnesium sulfate, and concentrated. The residue was purified by silica gel column chromatography (ethyl acetate-hexane) to obtain the desired product (27 mg) as a solid.

¹H NMR (CDCl₃) δ 1.66 (3H, d), 2.12 (3H, s), 2.25 (1H, t), 2.42 (1H, d), 4.77 (2H, d), 5.22 (1H, dd), 5.44 (2H, d), 7.49 (2H, d), 7.56 (1H, d), 7.72 (2H, d), 7.94 (1H, d).

### EXAMPLE 42

### 2-Acetyl-1-{[6-chloro-5-(hydroxymethyl)pyridin-3-yl]methyl}-4-(4-cyanophenyl)-5-methyl-1H-pyrrole-3-carbonitrile (Compound 42)

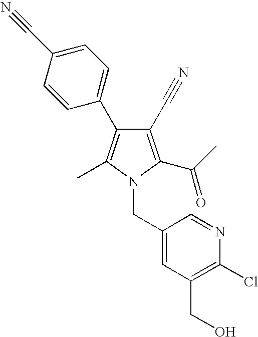

A 1N-solution (0.3 ml) of tetrabutylammonium fluoride in THF was added dropwise to a solution of 2-acetyl-1-{[5-{[(tert-butyl(dimethyl)silyl)]oxy}methyl]-6-chloropyridin-3-yl}-methyl}-4-(4-cyanophenyl)-5-methyl-1H-pyrrole-3-carbonitrile (90 mg) in THF (3 ml) at room temperature, and the mixture was stirred for 2 hours. The reaction mixture was poured into an aqueous saturated sodium chloride solution (10 ml), this was extracted with ethyl acetate, and the ethyl acetate layer was washed with an aqueous saturated sodium chloride solution, dried over magnesium sulfate, and concentrated. The residue was purified by silica gel column chromatography (ethyl acetate-hexane) to obtain the desired product (36 mg) as an oil.

¹H NMR (CDCl₃) δ 2.28 (3H, s), 2.48 (1H, br s), 2.76 (3H, s), 4.76 (2H, s), 5.70 (2H, s), 7.53 (2H, d), 7.63 (1H, d), 7.78 (2H, d), 7.97 (1H, d).

### EXAMPLE 43

### 1-{[6-Chloro-5-(hydroxymethyl)pyridin-3-yl]methyl}-4-(4-cyanophenyl)-2-(1-hydroxy-1-methylethyl)-5-methyl-1H-pyrrole-3-carbonitrile (Compound 43)

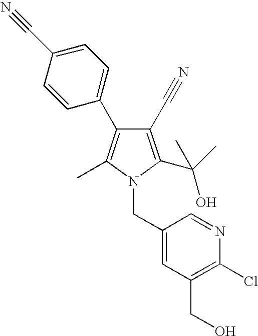

A 3N solution (0.1 ml) of methylmagnesium bromide in THF was added dropwise to a solution of Compound 42 (29 mg) in THF (3 ml) under ice-cooling, and the mixture was stirred for 2 hours under ice-cooling. The reaction mixture was poured into an aqueous saturated sodium chloride solution (10 ml), this was extracted with ethyl acetate, and the ethyl acetate layer was washed with an aqueous saturated sodium chloride solution, dried over magnesium sulfate, and concentrated. The residue was purified by silica gel column chromatography (ethyl acetate-hexane) to obtain the desired product (1.4 mg) as a solid.

¹H NMR (CDCl₃) δ 1.82 (6H, s), 2.05 (2H, br s), 2.06 (3H, s), 4.78 (2H, s), 5.65 (2H, s), 7.48 (2H, d), 7.53 (1H, d), 7.72 (2H, d), 7.86 (1H, d).

### EXAMPLE 44

### 1-{[6-Chloro-5-(hydroxymethyl)pyridin]-3-yl}methyl}-4-(cyanophenyl)-2-(3-hydroxyprop-1-en-1-yl)-5-methyl-1H-pyrrole-3-carbonitrile (Compound 44)

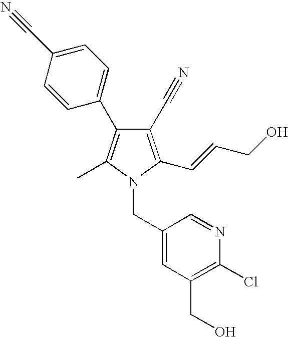

A mixture of the oil (45 mg) obtained by performing the same reaction as that shown in Example 1 from ethyl 2-chloro-5-({3-cyano-4-(4-cyanophenyl)-2-[(1E)-3-ethoxy-3-oxyprop-1-en-1-yl]-5-methyl-1H-pyrrol-1-yl}methyl)nicotinate (99 mg), tert-butyldimethylsilyl chloride (40 mg), imidazole (37 mg), and DMF was stirred at room temperature for 6 hours. The reaction mixture was poured into an aqueous saturated sodium chloride solution (10 ml), this was extracted with ethyl acetate, and the ethyl acetate layer was washed with an aqueous saturated sodium chloride solution, dried over magnesium sulfate, and concentrated. A 1N solution (0.2 ml) of tetrabutylammonium fluoride in THF was added dropwise to a solution of the resulting solid (27 mg) in THF (3 ml) at room temperature, and the mixture was stirred for 2 hours. The reaction mixture was poured into an aqueous saturated sodium chloride solution (10 ml), this was extracted with ethyl acetate, and the ethyl acetate layer was washed with an aqueous saturated sodium chloride solution, dried over magnesium sulfate, and concentrated. The residue was purified by silica gel column chromatography (ethyl acetate-hexane) to obtain the desired product (11 mg) as a solid.

¹H NMR (DMSO-D₆) δ2.21 (3H, s), 4.14 (2H, d), 4.51 (2H, d), 5.03 (1H, t), 5.42 (2H, s), 5.62 (1H, s), 6.66 (2H, s), 7.5-7.7 (3H, m), 7.9-8.0 (2H, m), 8.03 (1H, d).

### EXAMPLE 45

### 1-{[6-Chloro-5-(hydroxymethyl)pyridin]-3-yl}methyl}-4-(4-cyanophenyl)-2-(hydroxymethyl)-5-methyl-1H-pyrrole-3-carbonitorile (Compound 45)

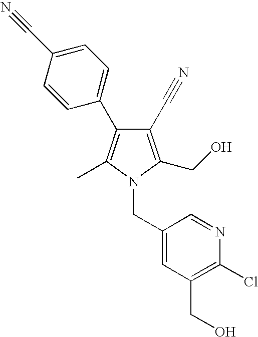

Sodium borohydride (0.17 g) was added to a solution of ethyl 2-chloro-5-{[3-cyano-4-(4-cyanophenyl)-2-formyl-5-methyl-1H-pyrrole-1-yl]methyl}nicotinate (0.37 g) in methanol (5 ml) at room temperature, and the mixture was stirred for 20 hours. The reaction mixture was poured into an aqueous saturated sodium chloride solution (20 ml), this was extracted with ethyl acetate, and the ethyl acetate layer was washed with an aqueous saturated solution, dried over magnesium sulfate, and concentrated. The residue was purified by silica gel column chromatography (ethyl acetate-hexane) to obtain the desired product (35 mg) as a solid.

¹H NMR (CDCl₃) δ 2.1-2.2 (2H, m), 2.20 (3H, s), 4.78 (2H, s), 4.79 (2H, s), 5.35 (2H, s), 7.50 (2H, d), 7.62 (1H, d), 7.73 (2H, d), 8.02 (1H, d).

### EXAMPLE 46

### 4-(1-{[6-Chloro-5-(hydroxymethyl)pyridin-3-yl]methyl}-2,5-dimethyl-1H-pyrrol-3-yl)benzonitrile (Compound 46)

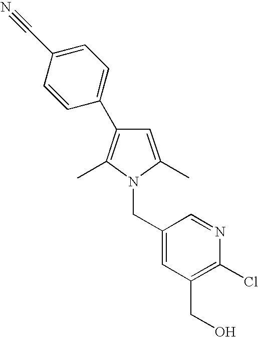

A mixture of sodium borohydride (113 mg), calcium chloride (333 mg), THF (14 ml) and ethanol (7 ml) was stirred at room temperature for 30 minutes, ethyl 2-chloro-5-{[3-(4-cyanophenyl)-2,5-dimethyl-1H-pyrrol-1-yl]methyl}benzoate (600 mg) was added thereto, and the mixture was stirred at room temperature for 14-hours. The reaction mixture was poured into an aqueous saturated citric acid solution (100 ml), and the mixture was extracted with ethyl acetate. The ethyl acetate layer was washed with a saturated sodium chloride solution, dried over magnesium sulfate, and concentrated. The residue was purified by silica gel column chromatography (ethyl acetate-hexane), and crystallized from diethyl ether to obtain the desired product (400 mg) as crystals. m.p. 128-129° C.

¹H NMR (CDCl₃) δ2.20 (3H, s), 2.27 (3H, s), 2.35-2.50 (1H, br), 4.74 (2H, s), 5.08 (2H, s), 6.11 (1H, s), 7.44 (2H, d), 7.49 (1H, d), 7.59 (2H, d), 7.89 (1H, d).

### EXAMPLE 47

### 4-(1-{[6-Chloro-5-(hydroxymethyl)pyridin-3-yl]methyl}-5-ethyl-2-methyl-1H-pyrrol-3-yl)benzonitrile (Compound 47)

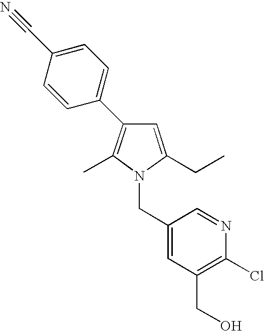

According to the same manner as that shown in Example 1, the desired compound was obtained by using ethyl 2-chloro-5-{[3-(4-cyanophenyl)-5-ethyl-2-methyl-1H-pyrrol-1-yl]methyl}nicotinate as starting material.

¹H NMR (CDCl₃) δ 1.25 (3H, t), 2.04 (1H, t), 2.28 (3H, s), 2.51 (2H, q), 4.75 (2H, d), 5.10 (2H, s), 6.14 (1H, s), 7.45-7.49 (3H, m), 7.61-7.65 (2H, m), 7.88-7.89 (1H, m).

### EXAMPLE 48

### 4-(1-{[6-Chloro-5-(hydroxymethyl)pyridin-3-yl]methyl}-5-cyclopropyl-2-methyl-1H-pyrrol-3-yl)benzonitrile (Compound 48)

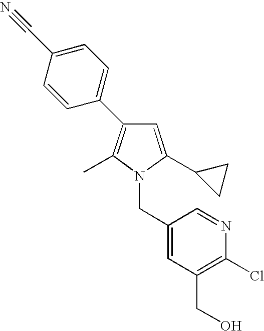

A mixture of sodium borohydride (46 mg), calcium chloride (100 mg), THF (4.0 ml) and ethanol (2.0 ml) was stirred at room temperature for 30 minutes, a mixture of ethyl 2-chloro-5-{[3-(4-cyanophenyl)-5-cyclopropyl-2-methyl-1H-pyrrol-1-yl]methyl}nicotinate and ethyl 2-chloro-5-{[4-(4-cyanophenyl)-2-cyclopropyl-5-methyl-1H-pyrrol-3-yl]methyl}nicotinate (253 mg) was added thereto, and the mixture was stirred at room temperature for 14 hours. The reaction mixture was poured into water (30 ml), and the mixture was extracted with ethyl acetate. The ethyl acetate layer was washed with a saturated sodium chloride solution, dried over magnesium sulfate, and concentrated. The residue was purified by silica gel column chromatography (ethyl acetate-hexane) to obtain the desired product (14.0 mg) as an amorphous solid.

¹H NMR (CDCl₃) δ: 0.59-0.62 (2H, m), 0.79-0.84 (2H, m), 1.52-1.60 (2H, m), 5.57 (3H, s), 4.76 (2H, s), 5.28 (2H, s), 6.03 (1H, s), 7.43 (2H, d), 7.52-7.53 (1H, m), 7.61 (2H, d), 7.96 (1H, d).

### EXAMPLE 49

### 4-(1-{[6-Chloro-5-(hydroxymethyl)pyridin-3-yl]methyl}-5-methyl-2-(trifluoromethyl)-1H-pyrrol-3-yl)benzonitrile (Compound 49)

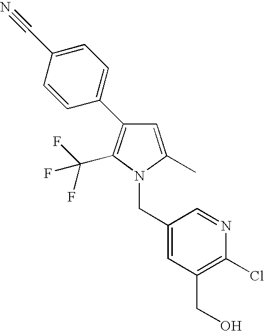

According to the same manner as that shown in Example 1, the desired compound was obtained by using ethyl 2-chloro-5-{[3-(4-cyanophenyl)-5-methyl-2-(trifluoromethyl)-1H-pyrrol-1-yl]methyl}nicotinate as starting material.

¹H NMR (CDCl₃) δ: 2.18 (4H, s), 4.78 (2H, s), 5.26 (2H, s), 6.06 (1H, s), 7.45 (2H, d), 7.54 (1H, d), 7.63-7.66 (2H, m), 7.90 (1H, d).

### EXAMPLE 50

### 4-(1-{[6-Chloro-5-(hydroxymethyl)pyridin-3-yl]methyl}-2-(hydroxymethyl)-5-methyl-1H-pyrrol-3-yl)benzonitrile (Compound 50)

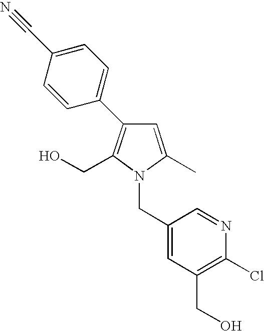

According to the same manner as that shown in Example 1, the desired compound was obtained by using ethyl 2-chloro-5-{[3-(4-cyanophenyl)-2-formyl-5-methyl-1H-pyrrol-1-yl]methyl}nicotinate as starting material.

¹H NMR (DMSO-d₆) δ: 2.13 (3H, s), 4.40 (2H, d), 4.49 (2H, d), 5.24 (1H, t), 5.32 (2H, s), 5.55 (1H, t), 7.61 (1H, d), 7.64 (2H, d), 7.80 (2H, d), 7.95 (1H, d).

### EXAMPLE 51

### 4-(1-{[6-Chloro-5-(hydroxymethyl)pyridin-3-yl]methyl}-2-(1-hydroxyethyl)-5-methyl-1H-pyrrol-3-yl)benzonitrile (Compound 51)

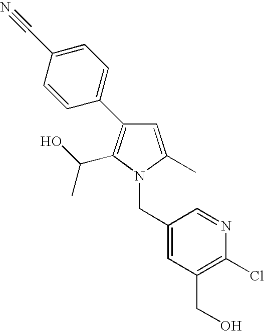

According to the same manner as that shown in Example 1, the desired compound was obtained by using ethyl 2-chloro-5-{[2-acetyl-3-(4-cyanophenyl)-5-methyl-1H-pyrrol-1-yl]methyl}nicotinate as starting material.

¹H NMR (CDCl₃): 1.48 (3H, d), 2.02-2.08 (4H, m), 4.73 (2H, s), 5.19 (1H, q), 5.34-5.53 (2H, m), 6.04 (1H, s), 7.39 (2H, d), 7.49 (1H, m), 7.62 (2H, d), 7.81-7.83 (1H, m).

### EXAMPLE 52

### 1-{[6-Chloro-5-(hydroxymethyl)pyridin-3-yl]methyl}-3-(4-cyanophenyl)-5-methyl-1H-pyrrole-2-carbonitrile (Compound 52)

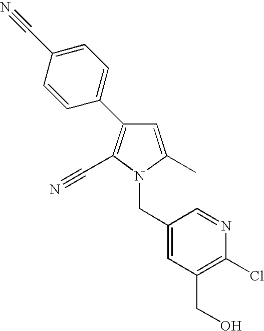

A mixture of sodium borohydride (64 mg), calcium chloride (70 mg), THF (3.0 ml) and ethanol (1.5 ml) was stirred at room temperature for 30 minutes, ethyl 2-chloro-5-{[2-cyano-3-(4-cyanophenyl)-5-methyl-1H-pyrrol-1-yl]methyl}nicotinate (170 mg) was added thereto, and the mixture was stirred at room temperature for 14 hours. The reaction mixture was poured into water, and the mixture was extracted with ethyl acetate. The ethyl acetate layer was washed with a saturated sodium chloride solution, dried over magnesium sulfate, and concentrated. The residue was purified by silica gel column chromatography (ethyl acetate-hexane), and crystallized from ethyle acetate-diisopropyl ether to obtain the desired product (139 mg) as crystals. m.p. 183.5-184.5° C.

¹H NMR (DMSO-d₆): 2.30 (3H, s), 4.51 (2H, d), 5.44 (2H, s), 5.63 (1H, t), 6.64 (1H, s), 7.65-7.66 (1H, m), 7.86 (2H, d), 7.92-7.95 (2H, m), 8.17 (1H, d).

### EXAMPLE 53

### 1-{[6-Chloro-5-(hydroxymethyl)pyridin-3-yl]methyl}-4-(4-cyano-2-fluorophenyl)-2,5-dimethyl-1H-pyrrole-3-carbonitrile (Compound 53)

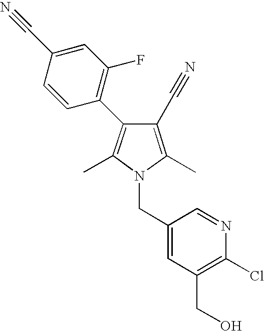

According to the same manner as that shown in Example 1, the desired compound was obtained by using ethyl 2-chloro-5-{[3-(4-cyano-2-fluorophenyl)-2,5-dimethyl-1H-pyrrol-1-yl]methyl}nicotinate as starting material.

¹H NMR (CDCl₃): 2.09 (1H, br s), 2.21 (3H, s), 2.40 (3H, s), 4.79 (2H, s), 5.14 (2H, s), 7.32 (1H, dd), 7.38 (1H, dt), 7.45 (1H, s), 7.51 (1H, d), 7.97 (1H, d).

### EXAMPLE 54

### Tert-butyl 1-{[6-chloro-5-(hydroxymethyl)pyridin-3-yl]methyl}-2-cyano-4-(4-cyanophenyl)-5-methyl-1H-pyrrole-3-carboxylate (Compound 54)

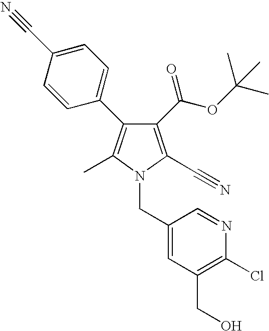

According to the same manner as that shown in Example 1, the desired compound was obtained by using ethyl 5-{[3-(tert-butoxycarbonyl)-2-cyano-4-(4-cyanophenyl)-5-methyl-1H-pyrrol-1-yl]methyl}-2-chloronicotinate as starting material.

¹H NMR (CDCl₃): 1.44 (9H, s), 2.11 (3H, s), 2.26 (1H, br s), 4.80 (2H, d), 5.33 (2H, s), 7.34 (2H, d), 7.68 (2H, d), 7.76 (1H, d), 8.12 (1H, d).

### EXAMPLE 55

### Tert-butyl 1-{[6-chloro-5-(hydroxymethyl)pyridin-3-yl]methyl}-4-(4-cyanophenyl)-5-methyl-2-(trifluoromethyl)-1H-pyrrole-3-carboxylate (Compound 55)

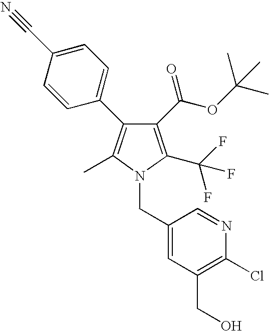

According to the same manner as that shown in Example 1, the desired compound was obtained by using ethyl 5-{[3-(tert-butoxycarbonyl)-4-(4-cyanophenyl)-5-methyl-2-(trifluoromethyl)-1H-pyrrol-1-yl]methyl}-2-chloronicotinate as starting material.

¹H NMR (CDCl₃): 1.37 (9H, s), 2.09 (3H, s), 2.14 (1H, br s), 4.78 (2H, d), 5.28 (2H, s), 7.38 (2H, m), 7.58 (1H, d), 7.68 (2H, m), 7.97 (1H, d).

### EXAMPLE 56

### 4-[(2-Chloro-5-{[3-(4-cyanophenyl)-2,5-dimethyl-1H-pyrrol-1-yl]methyl}pyridin-3-yl)methoxy]-4-oxobutanoic acid (Compound 56)

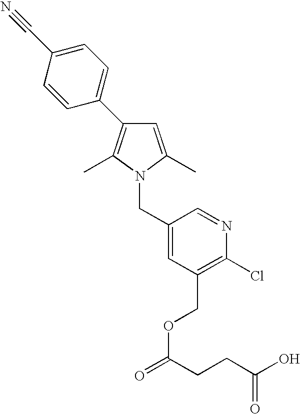

A mixture of Compound 46 (222 mg), 4-tert-butoxy-4-oxobotanoic acid (122 mg), WSC (159 mg) and pyridine (2 ml) was stirred at room temperature for 7 hours. WSC (80 mg) was added thereto and the mixture was stirred at 65° C. for 16 hours. The reaction mixture was concentrated and distributed between ethyl acetate and water. The organic layer was washed with an aqueous 10% citric acid solution and an aqueous sodium bicarbonate solution, dried over sodium sulfate, and concentrated. The residue was purified by silica gel column chromatography (ethyl acetate-hexane) to obtain 2-chloro-5-{[3-cyano-4-(4-cyanophenyl)-2,5-dimethyl-1H-pyrrol-1-yl]methyl}benzyl tert-butyl succinate (200 mg). To a mixture of this compound (200 mg) and acetonitrile (2 ml) was added trifluoroacetic acid (2 ml), and the mixture was stirred at room temperature for 1 hour. The reaction mixture was concentrated, and ethyl acetate and an aqueous sodium bicaronate solution were added thereto. The mixture was neutralized with an aqueous 10% citric acid solution and separated into layers. The organic layer was dried over sodium sulfateand concentrated. The residue was crystallized from ethyl acetate to obtain the desired product (150 mg) as crystals.

m.p. 201-204° C.

¹H NMR (CDCl₃): 2.20 (3H, s), 2.27 (3H, s), 2.66 (4H, s), 5.09 (2H, s), 5.20 (2H, s), 6.15 (1H, s), 7.22 (1H, s), 7.46 (2H, d), 7.63 (2H, d), 8.01 (1H, s).

### EXAMPLE 57

### 4-[(2-Chloro-5-{[3-cyano-4-(4-cyanophenyl)-2,5-dimethyl-1H-pyrrol-1-yl]methyl}pyridin-3-yl)methoxy]-4-oxobutanoic acid (Compound 57)

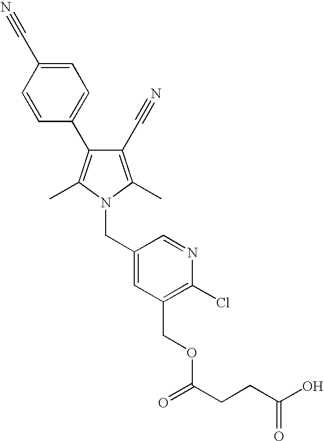

A mixture of Compound 1 (100 mg), siccinic anhydride (53 mg) and pyridine (2 ml) was stirred at 90° C. for 13 hours. The reaction mixture was cooled to room temperature and poured into an aqueous 5% citric acid solution, and the mixture was extracted with ethyl acetate. The extract was washed with an aqueous 5% citric acid solution and water, dried over sodium sulfate and concentrated. The residue was crystallized from ethyl acetate to obtain the desired product (105 mg) as crystals. m.p. 187-188° C.

¹H NMR (CDCl₃) δ: 2.20 (3H, s), 2.39 (3H, s), 2.67-2.70 (4H, m), 5.13 (2H, s), 5.21 (1H, s), 7.30 (1H, d), 7.49-7.53 (2H, m), 7.69-7.72 (2H, m), 7.99 (1H, d).

### EXAMPLE 58

### 1-{[6-Chloro-5-(hydroxymethyl)pyridin-3-yl]methyl}-4-(4-cyanophenyl)-2,5-dimethyl-1H-pyrrole-3-carbonitrile benzenesulfonate (Compound 58)

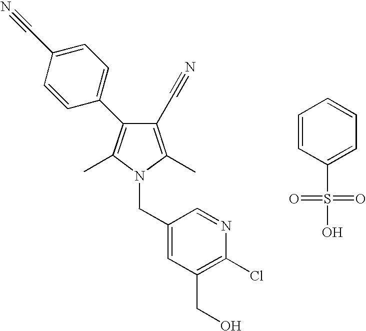

To a mixture of Compound 1 (100 mg), tetrahydrofuran (2 ml) and diethyl ether (2 ml) was added benzenesulfonic acid monohydrate (47 mg) at room temperature. After stirring for 17 hours, a precipitate was collected by filtration and washed with diethyl ether to obtain a mono-ethanol solvate of the desired product (118 mg) as a solid.

m.p. 179-181° C. (decomp.).

¹H NMR (DMSO-d₆): 1.06 (3H, t), 2.21 (3H, s), 2.37 (3H, s), 3.44 (2H, q), 4.52 (2H, s), 5.37 (2H, s), 7.29-7.36 (3H, m), 7.50-7.65 (5H, m), 7.92 (2H, d), 8.06 (1H, d)

### EXAMPLE 59

### 1-{[6-Chloro-5-(hydroxymethyl)pyridin-3-yl]methyl}-4-(4-cyanophenyl)-2-ethyl-5-methyl-1H-pyrrole-3-carbonitrile benzenesulfonate (Compound 59)

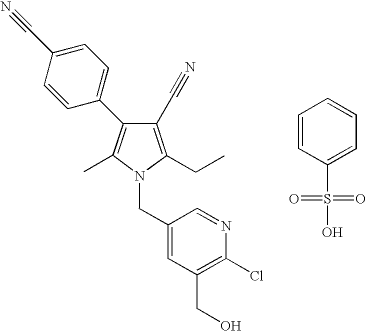

To a mixture of Compound 2 (58 mg), tetrahydrofuran (0.4 ml) and diethyl ether (0.4 ml) was added a mixture of benzenesulfonic acid monohydrate (26 mg), tetrahydrofuran (0.15 ml) and diethyl ether (0.15 ml) at room temperature. After stirring for 17 hours, a precipitate was collected by filtration and washed with diethyl ether to obtain the desired product (73 mg) as a solid. m.p. 174-175° C.

¹H NMR (DMSO-d₆): 1.11 (3H, t), 2.17 (3H, s), 2.78 (2H, q), 4.52 (2H, s), 5.40 (2H, s), 7.30-7.36 (3H, m), 7.56-7.62 (5H, m), 7.91-7.94 (2H, m), 8.01 (1H, d).

### EXAMPLE 60

### 1-{[6-Chloro-5-(hydroxymethyl)pyridin-3-yl]methyl}-4-(4-cyanophenyl)-5-methyl-2-propyl-1H-pyrrole-3-carbonitrile benzenesulfonate (Compound 60)

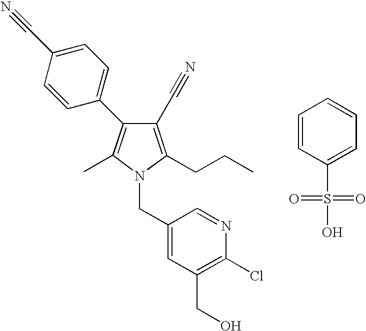

To a mixture of Compound 3 (3.0 g) and ethyl acetate (20 ml) was added benzenesulfonic acid monohydrate (1.31 g) at room temperature. After stirring for 17 hours, a precipitate was collected by filtration and washed with diethyl ether to obtain the desired product (3.44 g) as a solid. m.p. 152-153° C.

¹H NMR (DMSO-d₆): 0.90 (3H, t), 1.43-1.56 (2H, m), 2.16 (3H, s), 2.74 (2H, t), 4.52 (2H, s), 5.40 (2H, s), 7.29-7.35 (3H, m), 7.55 (1H, d), 7.58 (1H, s), 7.58-7.62 (3H, m), 7.92 (2H, d), 8.00 (1H, d).

### EXAMPLE 61

### 1-{[6-Chloro-5-(hydroxymethyl)pyridin-3-yl]methyl}-4-(4-cyanophenyl)-2-cyclopropyl-5-methyl-1H-pyrrole-3-carbonitrile benzenesulfonate (Compound 61)

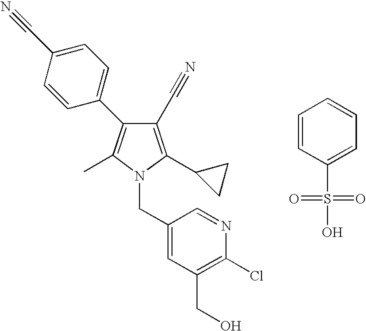

To a mixture of Compound 5 (100 mg), tetrahydrofuran (2.0 ml) and ethyl acetate (2.0 ml) was added a mixture of benzenesulfonic acid monohydrate (43.7 mg), tetrahydrofuran (0.1 ml) and diethyl ether (0.1 ml) at room temperature. The mixture was stirred for 18 hours. The solvent was concentrated under reduced pressure and to the residue were added tetrahydrofuran (0.25 ml) and diethyl ether (0.5 ml). A precipitate formed was collected by filtration and washed with diethyl ether to obtain the desired product (131 mg) as a solid. m.p. 184.5-185.5° C.

¹H NMR (DMSO-d₆): 0.80-0.85 (2H, m), 0.96-1.01 (2H, m), 1.73-1.91 (2H, m), 2.17 (3H, s), 4.52 (2H, s), 5.48 (2H, s), 7.30-7.33 (3H, m), 7.54-7.61 (6H, m), 7.90 (2H, d), 7.80 (1H, d).

### EXAMPLE 62

### 1-{[6-Chloro-5-(hydroxymethyl)pyridin-3-yl]methyl}-4-(4-cyanophenyl)-2,5-dimethyl-1H-pyrrole-3-carbonitrile methanesulfonate (Compound 62)

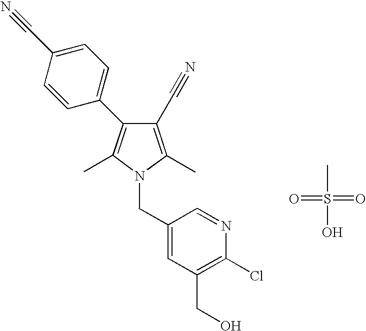

To a mixture of Compound 1 (100 mg) and ethyl acetate (2 ml) was added methanesulfonic acid (0.017 ml) at room temperature, and the mixture was stirred for 20 hours. A precipitate was collected by filtration and washed with ethyl acetate to obtain the desired product (110 mg) as a solid. m.p. 108-111° C.

¹H NMR (DMSO-d₆) δ: 2.21 (3H, s), 2.35 (3H, s), 2.37 (3H, s), 4.52 (2H, s), 5.37 (2H, s), 7.56-7.63 (3H, m), 7.92 (2H, d), 8.06 (1H, d)

### EXAMPLE 63

### 1-{[6-Chloro-5-(hydroxymethyl)pyridin-3-yl]methyl}-4-(4-cyanophenyl)-2-ethyl-5-methyl-1H-pyrrole-3-carbonitrile methanesulfonate (Compound 63)

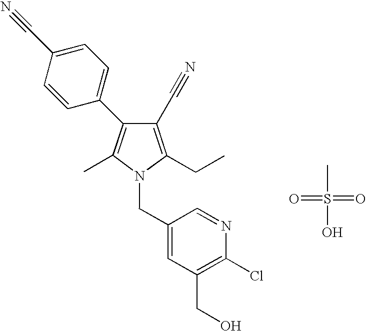

To a mixture of Compound 2 (98 mg) and ethyl acetate (0.75 ml) was added methanesulfonic acid (0.016 ml) at 40° C. The mixture was cooled to room temperature, and stirred for 9 hours. A precipitate was collected by filtration and washed with ethyl acetate to obtain the desired product (105 mg) as a solid. m.p. 154-155° C.

¹H NMR (DMSO-d₆) δ: 1.11 (3H, t), 2.17 (3H, s), 2.35 (3H, s), 2.78 (2H, q), 4.52 (2H, s), 5.40 (2H, s), 7.55-7.60 (3H, m), 7.90-7.94 (2H, m), 8.01 (1H, d).

### EXAMPLE 64

### 1-{[6-Chloro-5-(hydroxymethyl)pyridin-3-yl]methyl}-4-(4-cyanophenyl)-5-methyl-2-propyl-1H-pyrrole-3-carbonitrile methanesulfonate (Compound 64)

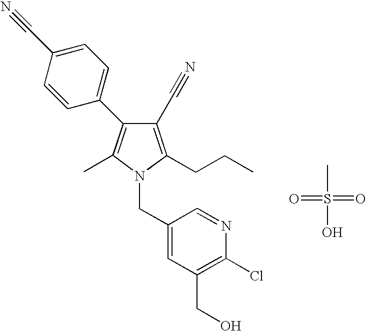

To a mixture of Compound 3 (100 mg) and ethyl acetate (0.8 ml) was added methanesulfonic acid (0.016 ml) at room temperature. After stirring for 14 hours, a precipitate was collected by filtration and washed with ethyl acetate to obtain the desired product (96 mg) as a solid. m.p. 157-158° C.

¹H NMR (DMSO-d₆) δ: 0.90 (3H, t), 1.49 (2H, sextet), 2.16 (3H, s), 2.39 (3H, s), 2.74 (2H, t), 4.51 (2H, s), 5.40 (2H, s), 7.54-7.60 (3H, m), 7.91 (2H, d), 7.98-7.99 (1H, m).

### EXAMPLE 65

### 1-{[6-Chloro-5-(hydroxymethyl)pyridin-3-yl]methyl}-4-(4-cyanophenyl)-2-cyclopropyl-5-methyl-1H-pyrrole-3-carbonitrile methanesulfonate (Compound 65)

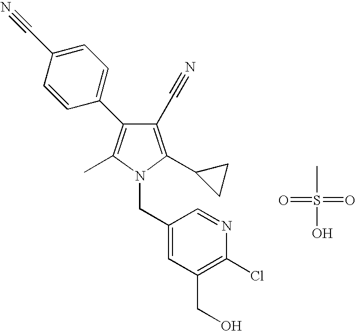

To a mixture of Compound 5 (100 mg) and ethyl acetate (1.0 ml) was added methanesulfonic acid (0.016 ml) at 50° C., and the mixture was cooled to room temperature and stirred for 16 hours. To the reaction mixture was added diethyl ether (1.0 ml), and the mixture was stirred at room temperature for 4 hours. A precipitate was collected by filtration and washed with ethyl acetate to obtain the desired product (68.4 mg) as a solid. m.p. 181.5-182.5° C.

¹H NMR (DMSO-d₆) δ: 0.80-0.86 (2H, m), 0.95-1.00 (2H, m), 1.81-1.87 (1H, m), 2.18 (3H, s), 2.41 (3H, s), 4.52 (2H, s), 5.48 (2H, s), 7.18 (1H, br s), 7.56 (2H, d), 7.59 (1H, d), 7.90 (2H, d), 8.08 (1H, d).

Compounds obtained in the aforementioned Examples are summarized in the following Table 1. Table 1

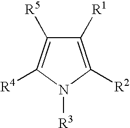

| Example | | | | | | |
|---|---|---|---|---|---|---|
| No. | R¹ | R² | R³ | | R⁴ | R⁵ |
| 1 | CN | Me | 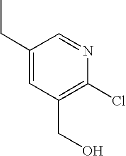 | | Me | 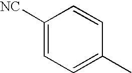 |
| 2 | CN | Et | 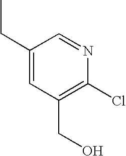 | | Me | 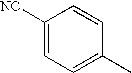 |
| 3 | CN | 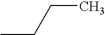 | 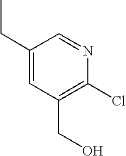 | | Me | 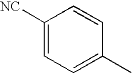 |
| 4 | CN | 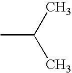 | 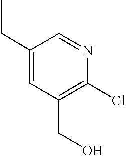 | | Me | 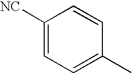 |
| 5 | CN | 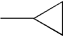 | 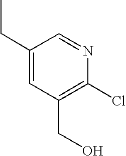 | | Me | 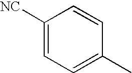 |
| 6 | CN | Me | 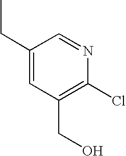 | | Me | 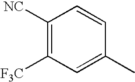 |
| 7 | CN | Me | 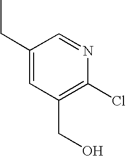 | | Me | 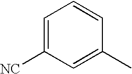 |
| 8 | CN | Me | 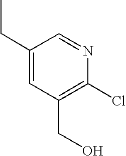 | | Et | 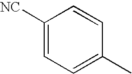 |
| 9 | CO₂Me | CF3 | 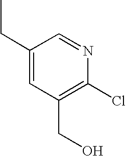 | | Me | 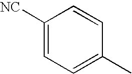 |
| 10 | CN | Me | 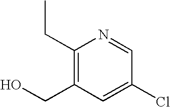 | | Me | 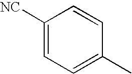 |
| 11 | CN | Me | 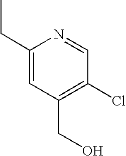 | | Me | 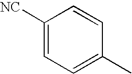 |
| 12 | CN | Me | 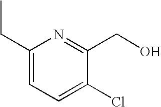 | | Me | 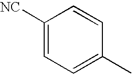 |
| 13 | CN | H | 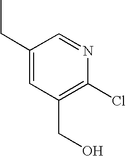 | | Me | 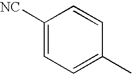 |
| 14 | CN | 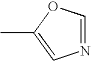 | 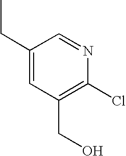 | | Me | 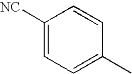 |
| 15 | CN | 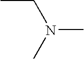 | 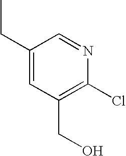 | | Me | 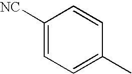 |
| 16 | CN | 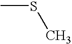 | 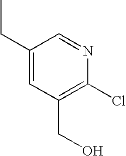 | | Me | 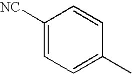 |
| 17 | CN | 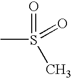 | 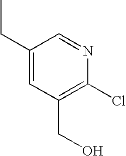 | | Me | 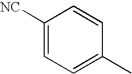 |
| 18 | CN | CF₃ | 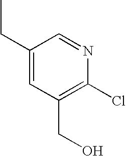 | | Me | 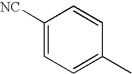 |
| 19 | CN | 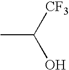 | 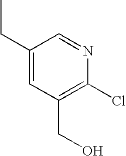 | | Me | 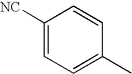 |
| 20 | CN | 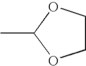 | 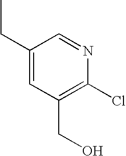 | | Me | 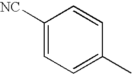 |
| 21 | CN | 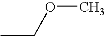 | 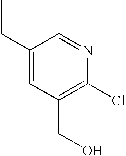 | | Me | 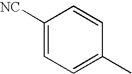 |
| 22 | CN | 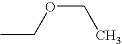 | 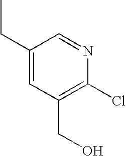 | | Me | 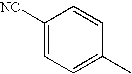 |
| 23 | CN | 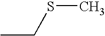 | 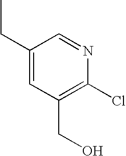 | | Me | 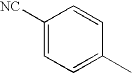 |
| 24 | CN | 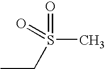 | 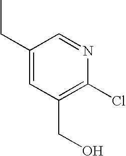 | | Me | 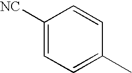 |
| 25 | CN | Me | 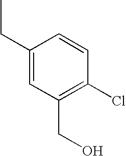 | | Me | 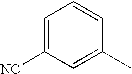 |
| 26 | CN | Me | 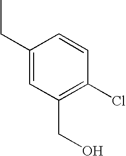 | | Me | 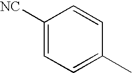 |
| 27 | CN | Me | 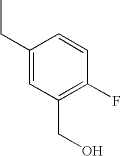 | | Me | 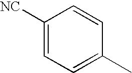 |
| 28 | CN | Et | 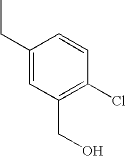 | | Me | 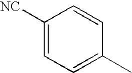 |
| 29 | CN | Me | 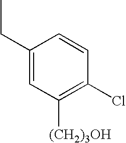 | | Me | 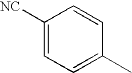 |
| 30 | CN | 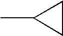 | 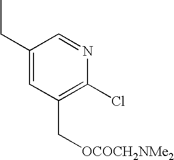 | | Me | 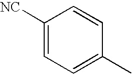 |
| 31 | CN | Me | 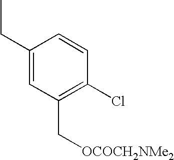 | | Me | 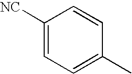 |
| 32 | CN | Me | 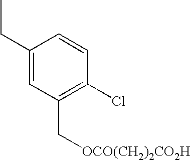 | | Me | 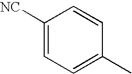 |
| 33 | CN | 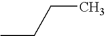 | 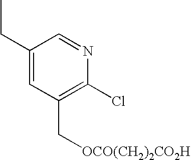 | | Me | 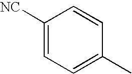 |
| 34 | CN | Et | 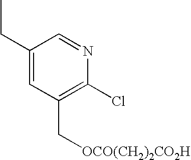 | | Me | 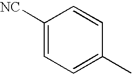 |
| 35 | CN | 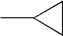 | 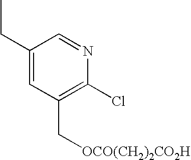 | | Me | 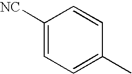 |
| 36 | CN | Me | 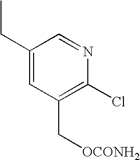 | | Me | 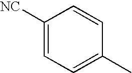 |
| 37 | CN | Me | 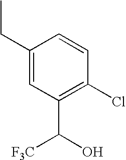 | | Me | 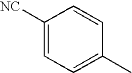 |
| 38 | CN | Me | 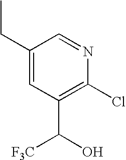 | | Me | 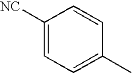 |
| 39 | CN | Me | 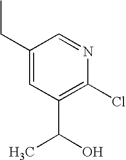 | | Me | 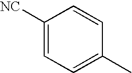 |
| 40 | CN | 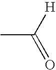 | 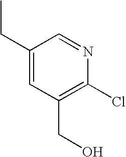 | | Me | 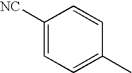 |
| 41 | CN | 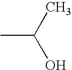 | 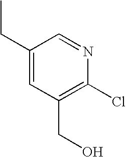 | | Me | 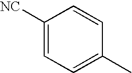 |
| 42 | CN | 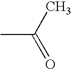 | 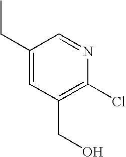 | | Me | 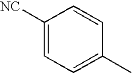 |
| 43 | CN | 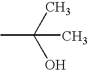 | 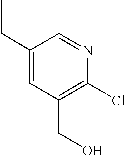 | | Me | 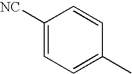 |
| 44 | CN | 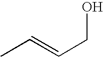 | 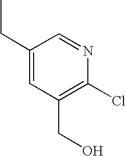 | | Me | 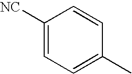 |
| 45 | CN | 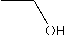 | 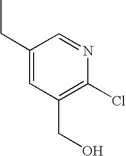 | | Me | 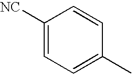 |
| 46 | H | Me | 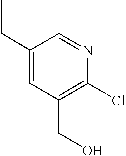 | | Me | 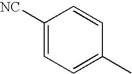 |
| 47 | H | Et | 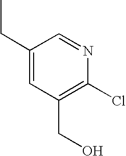 | | Me | 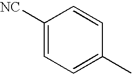 |
| 48 | H | 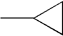 | 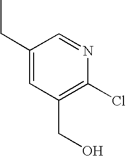 | | Me | 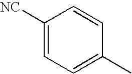 |
| 49 | H | Me | 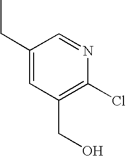 | | CF₃ | 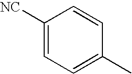 |
| 50 | H | Me | 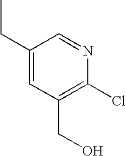 | | CH2OH | 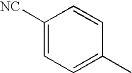 |
| 51 | H | Me | 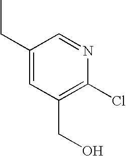 | | 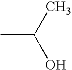 | 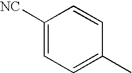 |
| 52 | H | Me | 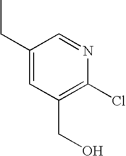 | | CN | 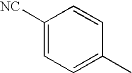 |
| 53 | CN | Me | 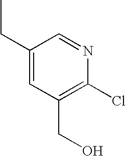 | | Me | 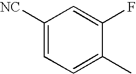 |
| 54 | CO₂tBu | CN | 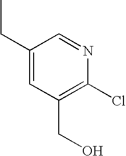 | | Me | 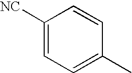 |
| 55 | CO₂tBu | CF₃ | 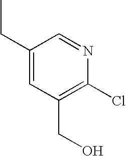 | | Me | 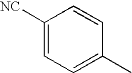 |
| 56 | H | Me | 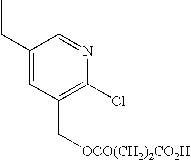 | | Me | 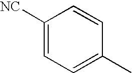 |
| 57 | CN | Me | 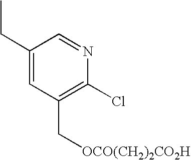 | | Me | 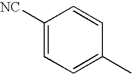 |
| 58 | CN | Me | 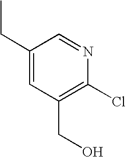 | 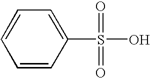 | Me | 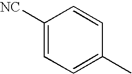 |
| 59 | CN | Et | 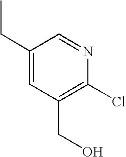 | 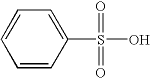 | Me | 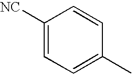 |
| 60 | CN | 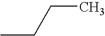 | 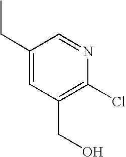 | 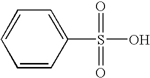 | Me | 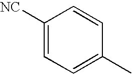 |
| 61 | CN | 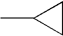 | 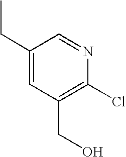 | 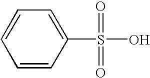 | Me | 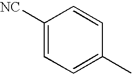 |
| 62 | CN | Me | 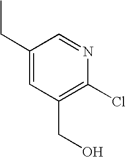 | 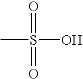 | Me | 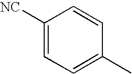 |
| 63 | CN | Et | 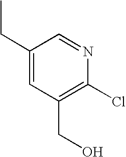 | 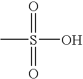 | Me | 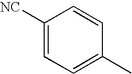 |
| 64 | CN | 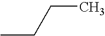 | 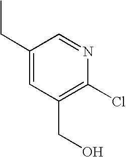 | 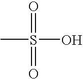 | Me | 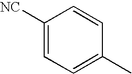 |
| 65 | CN | 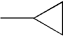 | 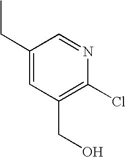 | 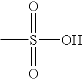 | Me | 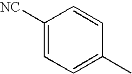 |

### PREPARATION EXAMPLE 1

| | | |
|---|---|---|
| (1) Compound of Example 1 | 5.0 | mg |
| (2) Sodium chloride | 20.0 | mg |
| (3) Distilled water | ad. 2 | mL |

5.0 Milligram of the compound of Example 1 and 20.0 mg of sodium chloride are dissolved in distilled water, and water is added to a total amount of 2.0 mL. The solution is filtered, and filled into a 2 mL ampoule under the sterile condition. The ampoule is sterilized, and sealed to obtain a solution for injection.

### PREPARATION EXAMPLE 2

| | | |
|---|---|---|
| (1) Compound of Example 1 | 50 | mg |
| (2) Lactose | 34 | mg |
| (3) Corn starch | 10.6 | mg |
| (4) Corn starch (paste) | 5 | mg |
| (5) Magnesium stearate | 0.4 | mg |
| (6) Calcium carboxymethylcellulose | 20 | mg |
| Total | 120 | mg |

According to a conventional method, (1) to (6) were mixed, and compressed with a tabletting machine to obtain tablets.

### PREPARATION EXAMPLE 3

| | | |
|---|---|---|
| (1) Compound of Example 7 | 5.0 | mg |
| (2) Sodium chloride | 20.0 | mg |
| (3) Distilled water | ad. 2 | mL |

5.0 Milligram of the compound of Example 7 and 20.0 mg of sodium chloride are dissolved in distilled water, and water is added to a total amount of 2.0 mL. The solution is filtered, and filled into a 2 mL ampoule under the sterile condition. The ampoule is sterilized, and sealed to obtain a solution for injection.

### PREPARATION EXAMPLE 4

| | | |
|---|---|---|
| (1) Compound of Example 7 | 50 | mg |
| (2) Lactose | 34 | mg |
| (3) Corn starch | 10.6 | mg |
| (4) Corn starch (paste) | 5 | mg |
| (5) Magnesium stearate | 0.4 | mg |
| (6) Calcium carboxymethylcellulose | 20 | mg |
| Total | 120 | mg |

According to the conventional method, (1) to (6) were mixed, and compressed with a tabletting machine to obtain tablets.

### PREPARATION EXAMPLE 5

| | | |
|---|---|---|
| (1) Compound of Example 9 | 5.0 | mg |
| (2) Sodium chloride | 20.0 | mg |
| (3) Distilled water | ad. 2 | mL |

5.0 Milligram of the compound of Example 9 and 20.0 mg of sodium chloride are dissolved in distilled water, and water is added to a total amount of 2.0 mL. The solution is filtered and filled into 2 ml ampoule under the sterile condition. The ampoule is sterilized and sealed to obtain a solution for injection.

### PREPARATION EXAMPLE 6

| | | |
|---|---|---|
| (1) Compound of Example 9 | 50 | mg |
| (2) Lactose | 34 | mg |
| (3) Corn starch | 10.6 | mg |
| (4) Corn starch (paste) | 5 | mg |
| (5) Magnesium stearate | 0.4 | mg |
| (6) Calcium carboxymethylcellulose | 20 | mg |
| Total | 120 | mg |

According to the conventional method (1) to (6) were mixed, and compressed with a tabletting machine to obtain tablets.

### TEST EXAMPLE 1

### AR Binding Inhibitory Test (Wild Type, LNCaP Type)

3 nM miboleron and 1 μM of a compound was added to a solution containing a wild-type androgen receptor (AR) or an androgen receptor (AR) having a LNCaP type mutation, this was incubated at 4° C. for 3 hours, and B(Bound)/F(Free) separation was performed by a dextran/charcoal method. A label count of B was measured, and an inhibition rate of the compound was calculated. The results are shown in Table 2.

TABLE 2
Inhibitory rate (%) at 1 μM

| Compound No. | Wild type | LNCaP type |
|---|---|---|
| 1 | 94 | 87 |
| 2 | 99 | 94 |
| 3 | 96 | 99 |
| 5 | 98 | 96 |
| 1 | 94 | 58 |
| 26 | 98 | 93 |
| 46 | 86 | 89 |
| 47 | 50 | 81 |
| 48 | 30 | 87 |
| bicalutamide | 88 | 63 |

### TEST EXAMPLE 2

### Test of Prostate Specific Antigen (PSA) Production Inhibition by Present Compound in Prostate Cancer Cell

A human prostate cancer cell LNCaP-FGC was inoculated on a 96-well plate at 5000 cells/100 μL/well and, on the next day, testosterone having the final concentration of 1 ng/mL and 1 μM of a compound were added. Three days after addition, the PSA concentration in the culture supernatant was measured by ELISA, and a PSA production inhibition rate was calculated, letting the rate of a testosterone non-added group to be 100%, and letting the rate of a testosterone-added group to be 0%. The results are shown in Table 3.

TABLE 3
Inhibitory rate at 1 μM

| Compound No. | Inhibitory rate (%) |
|---|---|
| 1 | 99 |
| 2 | 95 |
| 3 | 99 |
| 5 | 99 |
| 7 | 94 |
| 26 | 97 |
| 38 | 94 |
| 46 | 95 |
| 48 | 98 |
| 52 | 99 |
| bicalutamide | 58 |

### TEST EXAMPLE 3

### AR Transcription Inhibition Test

5,000,000 Cells of Cos-7 was inoculated on a flask, and cultured in a culturing solution (DMEM+10% Dextran Charcoal (DCC)-Fetal Bovine Serum (FBS)+2 mM glutamine) for 24 hours, and a vector DNA in which mutated AR (W741C) had been inserted, and a vector DNA in which a luciferase gene had been connected downstream of an androgen-responsive promoter were transfected by a liposome method. After two hours, the medium was exchanged, the cells were cultured for 3 hours, DHT (dihydrotestosterone) having the final concentration of 0.04 μM and an assessment compound having the final concentration of 0.1 μM were added, and the cells were cultured for 24 hours, and luciferase activity was measured to investigate AR transcription inhibition activity of the assessment compound. The results expressed by an inhibitory rate (%) relative to a control are shown in Table 4.

TABLE 4
Inhibitory rate at 0.1 μM

| Compound No. | Inhibitory rate (%) |
|---|---|
| 1 | 49 |
| 2 | 30 |
| 3 | 78 |
| 5 | 78 |
| 26 | 53 |
| Bicalutamide | −72 |

### TEST EXAMPLE 4

### Test of Reduction in Weight of Mouse Prostate

A compound (bicalutamide is 100 mg/kg/day) was orally administered to a 8 week old male mouse at 50 mg/kg/day for 7 days, and a weight of prostate was measured on the next date from completion of administration. A rate of reduction in a prostate weight is shown in Table 5, letting at drug non-addition to be 0%, and letting at castration to be 100%.

TABLE 5

| Compound No. | Reduction rate (%) |
|---|---|
| 1 | 74 |
| 38 | 68 |
| Bicalutamide | 40 |

### INDUSTRIAL APPLICABILITY

The compound (I) of the present invention or a salt thereof has excellent antagonism for a normal androgen receptor and/or a mutated androgen receptor, and is useful as an agent for preventing or treating a hormone-sensitive cancer at an androgen dependent term and/or at an androgen independent term.

1. A compound represented by the formula: 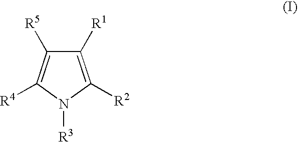
wherein R¹ represents a hydrogen atom, a cyano group or a group represented by the formula COOR^{A} (wherein R^{A} represents an optionally substituted C₁₋₆ alkyl group), R² and R⁴ are the same or different, and each represents a hydrogen atom, a C₁₋₆ alkyl group, a C₃₋₆ cycloalkyl group, a trifluoromethyl group, an amino-C₁₋₆ alkyl group, a mono- or di-substituted amino-C₁₋₆ alkyl group, an optionally halogenated C₁₋₆ alkyl group substituted with an optionally substituted hydroxyl group, a C₂₋₆ alkenyl group substituted with an optionally substituted hydroxyl group, a C₁₋₆ alkyl group substituted with an optionally substituted and optionally oxidized thiol group, an optionally substituted and optionally oxidized thiol group, a cyano group, an acyl group, an optionally substituted oxazolyl group or a 1,3-dioxolan-2-yl group, R³ represents a group represented by the formula: 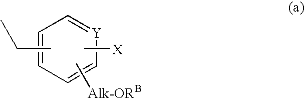
(wherein X represents a halogen atom, Y represents a carbon atom or a nitrogen atom, Alk represents an optionally substituted C₁₋₄ alkylene group, and R^{B} represents a hydrogen atom or an acyl group), and R⁵ represents a phenyl group which has a cyano group at a 4-position or a 3-position thereof and may be further substituted, or a salt thereof.

2. The compound according to claim 1, wherein R¹ is a cyano group.

3. The compound according to claim 1, wherein R² is a C₁₋₆ alkyl group, a C₃₋₆ cycloalkyl group, a trifluoromethyl group, an amino-C₁₋₆ alkyl group, a mono- or di-substituted amino-C₁₋₆ alkyl group, a C₁₋₆ alkyl group substituted with an optionally substituted hydroxyl group, a C₁₋₆ alkyl group substituted with an optionally substituted and optionally oxidized thiol group, an optionally substituted and optionally oxidized thiol group, a cyano group or an acyl group.

4. The compound according to claim 1, wherein R² is a C₁₋₆ alkyl group, a C₃₋₆ cycloalkyl group, a trifluoromethyl group, an amino-C₁₋₆ alkyl group, a mono- or di-substituted amino-C₁₋₆ alkyl group, a C₁₋₆ alkyl group substituted with an optionally substituted hydroxyl group, a C₁₋₆ alkyl group substituted with an optionally substituted and optionally oxidized thiol group, a cyano group or an acyl group.

5. The compound according to claim 3, wherein R² is a methyl group, an ethyl group, a propyl group, an isopropyl group, a cyclopropyl group, a trifluoromethyl group, a mono- or di-substituted amino-methyl group, a hydroxymethyl group, a hydroxyethyl group, a hydroxyisopropyl group, a C₁₋₆ alkyloxymethyl group, a C₁₋₆ alkylthio group, a C₁₋₆ alkylsulfonyl group, a C₁₋₆ alkylthiomethyl group, a C₁₋₆ alkylsulfonylmethyl group, an acetyl group, a carbamoyl group or a mono- or di-substituted carbamoyl group.

6. The compound according to claim 3, wherein R² is a methyl group, an ethyl group, a propyl group, an isopropyl group, a cyclopropyl group, a trifluoromethyl group, a mono- or di-substituted amino-methyl group, a hydroxymethyl group, a hydroxyethyl group, a hydroxyisopropyl group, a C₁₋₆ alkyloxymethyl group, a C₁₋₆ alkylthiomethyl group, a C₁₋₆ alkylsulfonylmethyl group, an acetyl group, a carbamoyl group or a mono- or di-substituted carbamoyl group.

7. The compound according to claim 1, wherein R³ is a group represented by the formula: 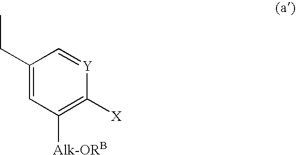
wherein each symbol is as defined in claim 1.

8. The compound according to claim 7, wherein X is a chlorine atom or a fluorine atom.

9. The compound according to claim 7, wherein R^{B} is a hydrogen atom, a succinoyl group or a dimethylaminomethylcarbonyl group.

10. The compound according to claim 7, wherein Alk is a methylene group.

11. The compound according to claim 1, wherein R⁴ is a C₁₋₆ alkyl group, a C₃₋₆ cycloalkyl group, a trifluoromethyl group, an amino-C₁₋₆ alkyl group, a mono- or di-substituted amino-C₁₋₆ alkyl group, a C₁₋₆ alkyl group substituted with an optionally substituted hydroxyl group, a C₁₋₆ alkyl group substituted with an optionally substituted and optionally oxidized thiol group, an optionally substituted and optionally oxidized thiol group, a cyano group or an acyl group.

12. The compound according to claim 1, wherein R⁴ is a methyl group, an ethyl group, a propyl group, an isopropyl group, a cyclopropyl group, a trifluoromethyl group, a mono- or di-substituted amino-methyl group, a hydroxymethyl group, a hydroxyethyl group, a hydroxyisopropyl group, a C₁₋₆ alkyloxymethyl group, a C₁₋₆ alkylthio group, a C₁₋₆ alkylsulfonyl group, a C₁₋₆ alkylthiomethyl group, a C₁₋₆ alkylsulfonylmethyl group, an acetyl group, a carbamoyl group or a mono- or di-substituted carbamoyl group.

13. The compound according to claim 1, wherein R⁵ is a 4-cyanophenyl group, a 3-cyanophenyl group, or a 4-cyano-3-(trifluoromethyl)phenyl group.

14. The compound according to claim 1, wherein R¹ is a hydrogen atom, a cyano group or a C₁₋₆ alkoxycarbonyl group,
R² is (i) a hydrogen atom,
(ii) an optionally halogenated C₁₋₆ alkyl group,
(iii) a C₃₋₆ cycloalkyl group,
(iv) a trifluoromethyl group,
(v) a C₁₋₆ alkyl group substituted with an amino mono- or di-substituted with C₁₋₆ alkyl,
(vi) an optionally halogenated C₁₋₆ alkyl group substituted with a hydroxyl group optionally substituted with C₁₋₆ alkyl,
(vii) a C₂₋₆ alkenyl group substituted with a hydroxyl group,
(viii) a C₁₋₆ alkyl group substituted with an optionally oxidized thiol group optionally substituted with C₁₋₆ alkyl,
(ix) an optionally oxidized thiol group optionally substituted with C₁₋₆ alkyl,
(x) a cyano group,
(xi) a C₁₋₆ alkanoyl group,
(xii) an oxazolyl group, or
(xiii) a 1,3-dioxolan-2-yl group,
Alk is a C₁₋₄ alkylene group optionally substituted with an optionally halogenated C₁₋₆ alkyl,
R^{B} is (i) a hydrogen atom, (ii) a carbamoyl group, (iii) a carboxy-C₁₋₆ alkyl-carbonyl group or (iv) a mono- or di-C₁₋₆ alkylamino-C₁₋₆ alkyl-carbonyl group,
X is a halogen atom,
Y is a carbon atom or a nitrogen atom,
R⁴ is a C₁₋₆ alkyl group optionally substituted with a hydroxyl group, a trifluoromethyl group, or a cyano group,
R⁵ is a phenyl group which has a cyano group at a 4-position or a 3-position thereof, and may be substituted with an optionally halogenated C₁₋₆ alkyl.

15. The compound according to claim 1, wherein R¹ is a hydrogen atom, a cyano group or a methoxycarbonyl group, R² is a methyl group, an ethyl group, a propyl group, an isopropyl group, a cyclopropyl group, a trifluoromethyl group, a 1-hydroxyethyl group or an acetyl group, R³ is a 6-chloro-5-(hydroxymethyl)pyridin-3-ylmethyl group, a 6-chloro-5-(1-hydroxy-2,2,2-trifluoroethyl)pyridin-3-ylmethyl group, a 4-chloro-3-(1-hydroxy-2,2,2-trifluoroethyl)benzyl group, a 4-chloro-3-(acetoxymethyl)benzyl group or a 4-chloro-3-(hydroxymethyl)benzyl group, R⁴ is a cyano group, a methyl group or an ethyl group, and R⁵ is a 4-cyano-2-fluorophenyl group or a 4-cyanophenyl group.

16. The compound according to claim 1, wherein R¹ is a hydrogen atom, a cyano group or a methoxycarbonyl group, R² is a methyl group, an ethyl group, a propyl group, a cyclopropyl group or a trifluoromethyl group, R³ is a 6-chloro-5-(hydroxymethyl)pyridin-3-ylmethyl group, a 6-chloro-5-(1-hydroxy-2,2,2-trifluoroethyl)pyridin-3-ylmethyl group, a 4-chloro-3-(1-hydroxy-2,2,2-trifluoroethyl)benzyl group, a 4-chloro-3-(acetoxymethyl)benzyl group or a 4-chloro-3-(hydroxymethyl)benzyl group, R⁴ is a cyano group, a methyl group or an ethyl group, and R⁵ is a 4-cyano-2-fluorophenyl group or a 4-cyanophenyl group.

17. A prodrug of the compound according to claim 1.

18. (i) 1-{[6-Chloro-5-(hydroxymethyl)pyridin-3-yl]methyl}-4-(4-cyanophenyl)-2,5-dimethyl-1H-pyrrole-3-carbonitrile,
(ii) 1-{[6-chloro-5-(hydroxymethyl)pyridin-3-yl]methyl}-4-(4-cyanophenyl)-2-ethyl-5-methyl-1H-pyrrole-3-carbonitrile,
(iii) 1-{[6-chloro-5-(hydroxymethyl)pyridin-3-yl]methyl}-4-(4-cyanophenyl)-5-methyl-2-propyl-1H-pyrrole-3-carbonitrile,
(iv) 1-{[6-chloro-5-(hydroxymethyl)pyridin-3-yl]methyl}-4-(4-cyanophenyl)-2-cyclopropyl-5-methyl-1H-pyrrole-3-carbonitrile,
(v) 1-{[6-chloro-5-(hydroxymethyl)pyridin-3-yl]methyl}-4-(3-cyanophenyl)-2,5-dimethyl-1H-pyrrole-3-carbonitrile,
(vi) 1-[4-chloro-3-(hydroxymethyl)benzyl]-4-(4-cyanophenyl)-2,5-dimethyl-1H-pyrrole-3-carbonitrile,
(vii) 2-chloro-5-{[3-cyano-4-(4-cyanophenyl)-2,5-dimethyl-1H-pyrrol-1-yl]methyl}benzyl N,N-dimethylglycinate,
(viii) 4-[(2-chloro-5-{[3-cyano-4-(4-cyanophenyl)-2,5-dimethyl-1H-pyrrol-1-yl]methyl}benzyl)oxy]-4-oxobutanoic acid,
(ix) 4-[(2-chloro-5-{[3-cyano-4-(4-cyanophenyl)-5-methyl-2-propyl-1H-pyrrol-1-yl]methyl}pyridin-3-yl)methoxy]-4-oxobutanoic acid,
(x) 4-[(2-chloro-5-{[3-cyano-4-(4-cyanophenyl)-2-ethyl-5-methyl-1H-pyrrol-1-yl]methyl}pyridin-3-yl)methoxy]-4-oxobutanoic acid,
(xi) 4-[(2-chloro-5-{[3-cyano-4-(4-cyanophenyl)-2-cyclopropyl-5-methyl-1H-pyrrol-1-yl]methyl}pyridin-3-yl)methoxy]-4-oxobutanoic acid,
(xii) 1-{[6-chloro-5-(2,2,2-trifluoro-1-hydroxyethyl)pyridin-3-yl]methyl}-4-(4-cyanophenyl)-2,5-dimethyl-1H-pyrrole-3-carbonitrile,
(xiii) 4-(1-{[6-chloro-5-(hydroxymethyl)pyridin-3-yl]methyl}-2,5-dimethyl-1H-pyrrol-3-yl)benzonitrile,
(xiv) 4-(1-{[6-chloro-5-(hydroxymethyl)pyridin-3-yl]methyl}-5-cyclopropyl-2-methyl-1H-pyrrol-3-yl)benzonitrile, or
(xv) 1-{[6-chloro-5-(hydroxymethyl)pyridin-3-yl]methyl}-3-(4-cyanophenyl)-5-methyl-1H-pyrrole-2-carbonitrile, or a salt thereof.

19. A process for preparing the compound according to claim 1 or a salt thereof, which comprises the steps of:
subjecting a compound represented by the formula: 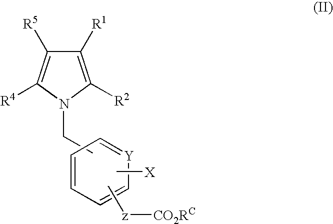
wherein Z represents a bond or an optionally substituted C₁₋₃ alkylene group, R^{C} represents an optionally substituted C₁₋₆ alkyl group, and the other symbols are as defined in claim 1, or a salt thereof to a reducing reaction to obtain a compound represented by the formula: 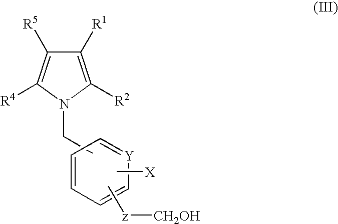
wherein the symbols are as defined above, or a salt thereof and, if necessary, subjecting it to a functional group-converting reaction.

20. A medicament comprising a compound represented by the formula: 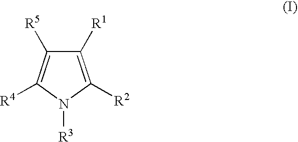
wherein R¹ represents a hydrogen atom, a cyano group or a group represented by the formula COOR^{A} (wherein R^{A} represents an optionally substituted C₁₋₆ alkyl group), R² and R⁴ are the same or different, and each represents a hydrogen atom, a C₁₋₆ alkyl group, a C₃₋₆ cycloalkyl group, a trifluoromethyl group, an amino-C₁₋₆ alkyl group, a mono- or di-substituted amino-C₁₋₆ alkyl group, an optionally halogenated C₁₋₆ alkyl group substituted with an optionally substituted hydroxyl group, a C₂₋₆ alkenyl group substituted with an optionally substituted hydroxyl group, a C₁₋₆ alkyl group substituted with an optionally substituted and optionally oxidized thiol group, an optionally substituted and optionally oxidized thiol group, a cyano group, an acyl group, an optionally substituted oxazolyl group or a 1,3-dioxolan-2-yl group, R³ represents a group represented by the formula: 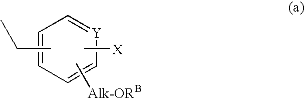
(wherein X represents a halogen atom, Y represents a carbon atom or a nitrogen atom, Alk represents an optionally substituted C₁₋₄ alkylene group, and R^{B} represents a hydrogen atom or an acyl group), and R⁵ represents a phenyl group which has a cyano group at a 4-position or a 3-position thereof and may be further substituted, or a salt or a prodrug thereof.

21. The medicament according to claim 20, which is an androgen receptor antagonist.

22. The medicament according to claim 20, wherein the androgen receptor is a normal androgen receptor and/or a mutated androgen receptor.

23. The medicament according to claim 20, which is an agent for preventing or treating hormone-sensitive cancer at an androgen dependent term and/or at an androgen independent term.

24. The medicament according to claim 20, which is an agent for preventing or treating prostate cancer.

25. A medicament which comprises a combination of a compound represented by the formula: 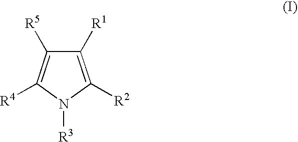
wherein R¹ represents a hydrogen atom, a cyano group or a group represented by the formula COOR^{A} (wherein R^{A} represents an optionally substituted C₁₋₆ alkyl group), R² and R⁴ are the same or different, and each represents a hydrogen atom, a C₁₋₆ alkyl group, a C₃₋₆ cycloalkyl group, a trifluoromethyl group, an amino-C₁₋₆ alkyl group, a mono- or di-substituted amino-C₁₋₆ alkyl group, an optionally halogenated C₁₋₆ alkyl group substituted with an optionally substituted hydroxyl group, a C₂₋₆ alkenyl group substituted with an optionally substituted hydroxyl group, a C₁₋₆ alkyl group substituted with an optionally substituted and optionally oxidized thiol group, an optionally substituted and optionally oxidized thiol group, a cyano group, an acyl group, an optionally substituted oxazolyl group or a 1,3-dioxolan-2-yl group, R³ represents a group represented by the formula: 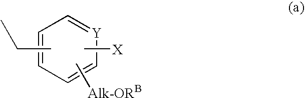
(wherein X represents a halogen atom, Y represents a carbon atom or a nitrogen atom, Alk represents an optionally substituted C₁₋₄ alkylene group, and R^{B} represents a hydrogen atom or an acyl group), and R⁵ represents a phenyl group which has a cyano group at a 4-position or a 3-position thereof and may be further substituted, or a salt or a prodrug thereof, and an anti-cancer drug.

26. The medicament according to claim 25, wherein the anti-cancer drug is an LH-RH derivative.

27. A method for antagonizing an androgen receptor of a mammal which comprises administering an effective amount of a compound represented by the formula: 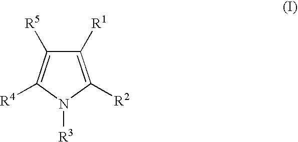
wherein R¹ represents a hydrogen atom, a cyano group or a group represented by the formula COOR^{A} (wherein R^{A} represents an optionally substituted C₁₋₆ alkyl group), R² and R⁴ are the same or different, and each represents a hydrogen atom, a C₁₋₆ alkyl group, a C₃₋₆ cycloalkyl group, a trifluoromethyl group, an amino-C₁₋₆ alkyl group, a mono- or di-substituted amino-C₁₋₆ alkyl group, an optionally halogenated C₁₋₆ alkyl group substituted with an optionally substituted hydroxyl group, a C₂₋₆ alkenyl group substituted with an optionally substituted hydroxyl group, a C₁₋₆ alkyl group substituted with an optionally substituted and optionally oxidized thiol group, an optionally substituted and optionally oxidized thiol group, a cyano group, an acyl group, an optionally substituted oxazolyl group or a 1,3-dioxolan-2-yl group, R³ represents a group represented by the formula: 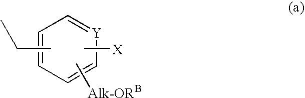
(wherein X represents a halogen atom, Y represents a carbon atom or a nitrogen atom, Alk represents an optionally substituted C₁₋₄ alkylene group, and R^{B} represents a hydrogen atom or an acyl group), and R⁵ represents a phenyl group which has a cyano group at a 4-position or a 3-position thereof and may be further substituted, or a salt or prodrug thereof to the mammal.

28. A method for preventing or treating hormone-sensitive cancer at an androgen dependent term and/or at an androgen independent term, which comprises administering an effective amount of a compound represented by the formula: 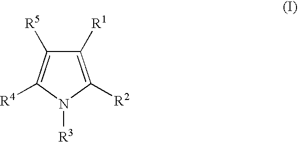
wherein R¹ represents a hydrogen atom, a cyano group or a group represented by the formula COOR^{A} (wherein R^{A} represents an optionally substituted C₁₋₆ alkyl group), R² and R⁴ are the same or different, and each represents a hydrogen atom, a C₁₋₆ alkyl group, a C₃₋₆ cycloalkyl group, a trifluoromethyl group, an amino-C₁₋₆ alkyl group, a mono- or di-substituted amino-C₁₋₆ alkyl group, an optionally halogenated C₁₋₆ alkyl group substituted with an optionally substituted hydroxyl group, a C₂₋₆ alkenyl group substituted with an optionally substituted hydroxyl group, a C₁₋₆ alkyl group substituted with an optionally substituted and optionally oxidized thiol group, an optionally substituted and optionally oxidized thiol group, a cyano group, an acyl group, an optionally substituted oxazolyl group or a 1,3-dioxolan-2-yl group, R³ represents a group represented by the formula: 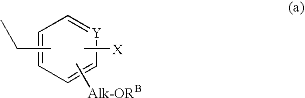
(wherein X represents a halogen atom, Y represents a carbon atom or a nitrogen atom, Alk represents an optionally substituted C₁₋₄ alkylene group, and R³ represents a hydrogen atom or an acyl group), and R⁵ represents a phenyl group which has a cyano group at a 4-position or a 3-position thereof and may be further substituted, or a salt or prodrug thereof to a mammal.

29. A method of preventing or treating prostate cancer, which comprises administering an effective amount of a compound represented by the formula: 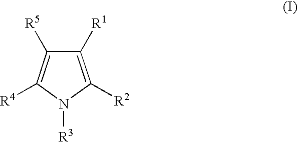
wherein R¹ represents a hydrogen atom, a cyano group or a group represented by the formula COOR^{A} (wherein R^{A} represents an optionally substituted C₁₋₆ alkyl group), R² and R⁴ are the same or different, and each represents a hydrogen atom, a C₁₋₆ alkyl group, a C₃₋₆ cycloalkyl group, a trifluoromethyl group, an amino-C₁₋₆ alkyl group, a mono- or di-substituted amino-C₁₋₆ alkyl group, an optionally halogenated C₁₋₆ alkyl group substituted with an optionally substituted hydroxyl group, a C₂₋₆ alkenyl group substituted with an optionally substituted hydroxyl group, a C₁₋₆ alkyl group substituted with an optionally substituted and optionally oxidized thiol group, an optionally substituted and optionally oxidized thiol group, a cyano group, an acyl group, an optionally substituted oxazolyl group or a 1,3-dioxolan-2-yl group, R³ represents a group represented by the formula: 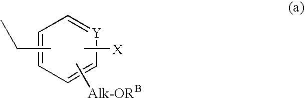
(wherein X represents a halogen atom, Y represents a carbon atom or a nitrogen atom, Alk represents an optionally substituted C₁₋₄ alkylene group, and R^{B} represents a hydrogen atom or an acyl group), and R⁵ represents a phenyl group which has a cyano group at a 4-position or a 3-position thereof and may be further substituted, or a salt or prodrug thereof to a mammal.

30. The method according to claim 28, wherein an effective amount of an anti-cancer drug is further administered.

31. The method according to claim 30, wherein the anti-cancer drug is an LH-RH derivative.

32-36. (canceled)

37. The method according to claim 29, wherein an effective amount of an anti-cancer drug is further administered.

38. The method according to claim 37, wherein the anti-cancer drug is an LH-RH derivative.
